(19) United States
(12) Patent Application Publication
(10) Pub. No.: US 2026/0195155 A1
(43) Pub. Date: Jul. 9, 2026

(54) SYSTEMS AND METHODS FOR MACHINE LEARNING-BASED TUNING OF PLATFORM PARAMETERS AND SETTINGS WITHIN SUBSCRIBER-SPECIFIC INSTANCES OF A DIGITAL PLATFORM

(71) Applicant: Technology Partners LLC d/b/a ImagineSoftware, Charlotte, NC (US)

(72) Inventor: Sam Faris Khashman, Charlotte, NC (US)

(73) Assignee: Technology Partners LLC d/b/a ImagineSoftware, Charlotte, NC (US)

(21) Appl. No.: 19/441,031

(22) Filed: Jan. 6, 2026

Related U.S. Application Data
(60) Provisional application No. 63/902,779, filed on Oct. 21, 2025.
(60) Provisional application No. 63/742,732, filed on Jan. 7, 2025.

(51) Int. Cl. G06F 9/455; G16H 70/20

(52) U.S. Cl. CPC G06F 9/455; G16H 70/20

(57) ABSTRACT

A system and method includes detecting an instance of a cloud-based service that has not been tuned during a predetermined time span, obtaining one or more corpora of subscriber-specific parameters characterizing a runtime behavior of the instance, converting the one or more corpora of subscriber-specific parameters into a plurality of distinct model input data objects operably configured for input into an ensemble of instance assessment machine learning models, generating, using the ensemble of instance assessment machine learning models, a proposed set of parameters for controlling a future runtime behavior of the instance, executing a computer-based simulation simulating an operation of the instance configured according to the proposed set of parameters, displaying, on a graphical user interface, the proposed set of parameters in association with simulation findings outputted by the computer-based simulation, and automatically executing an instance adaptation process that adapts the instance to an adapted instance.

### CROSS-REFERENCE TO RELATED APPLICATIONS

This application claims the benefit of U.S. Provisional Application No.: 63/902,779, filed on 21-OCT-2025, and U.S. Provisional Application No.: 63/742,732, filed on 07-JAN-2025, which are incorporated in their entireties by this reference.

### TECHNICAL FIELD

This invention relates generally to machine learning-based systems and methods, and more specifically, to a new and useful system and method for automated and semi-automated parameter tuning of subscriber-specific instances of a digital platform using one or more machine learning models.

### BACKGROUND

Conventional digital platforms (e.g., software applications, software services, etc.) are used by many subscribers. Each subscriber is typically provided a distinct instance of the digital platform and uses that instance without regard to how platform configuration parameters are defined or how those platform configuration parameters affect platform performance. As a result, subscribers often operate their instances with suboptimal platform configurations.

Therefore, there is a need in the art for systems and methods that automatically provide subscribers with platform tuning proposals to ensure subscriber-specific instances are optimally configured. Additionally, there is a further need in the art to automatically or semi-automatically implement the platform tuning proposals to reduce the amount of time the subscriber-specific instances remain in a suboptimal state. The embodiments of the present application provide technical solutions that address, at least, the needs described above, as well as deficiencies of the state of the art.

### BRIEF SUMMARY OF THE EMBODIMENTS

In one embodiment, a computer-implemented method for improving a computing performance of a subscriber-specific instance of a multi-tenant cloud-based service includes automatically detecting, in real-time using one or more processors, a target subscriber-specific instance of the multi-tenant cloud-based service that has not been tuned during a predetermined time span; in response to detecting that the target subscriber-specific instance has not been tuned during the predetermined time span, obtaining, in real-time from one or more computer databases, one or more corpora of subscriber-specific parameters characterizing a runtime behavior of the target subscriber-specific instance, wherein: at least a subset of the one or more corpora of subscriber-specific parameters controls a runtime of the target subscriber-specific instance; converting, in response to obtaining the one or more corpora of subscriber-specific parameters, the one or more corpora of subscriber-specific parameters into a plurality of distinct model input data objects operably configured for input into an ensemble of instance assessment machine learning models; generating, using the ensemble of instance assessment machine learning models, a proposed set of parameters for controlling a future runtime behavior of the target subscriber-specific instance based on providing the plurality of distinct model input data objects as input to the ensemble of instance assessment machine learning models; executing, in response to generating the proposed set of parameters, one or more computer-based simulations simulating an operation of the target subscriber-specific instance configured according to the proposed set of parameters; in response to executing the one or more computer-based simulations, displaying, on a graphical user interface, the proposed set of parameters in association with simulation findings outputted by the one or more computer-based simulations; receiving, while displaying the graphical user interface, an input from a user selecting a tune instance button displayed on the graphical user interface; and in response to receiving the input from the user selecting the tune instance button, automatically executing an instance adaptation process that adapts, in real-time or near real-time, the target subscriber-specific instance to an adapted subscriber-specific instance, wherein: the adapted subscriber-specific instance is configured according to the proposed set of parameters or a user-modified version of the proposed set of parameters, and all future automated digital actions associated with the user that occur within a target time span are executed using the adapted subscriber-specific instance instead of the target subscriber-specific instance.

In one embodiment, the one or more corpora of subscriber-specific parameters specifies a current threshold value for a respective minimum time-based threshold, the target subscriber-specific instance is configured to automatically route a subject digital item to a disposal queue when a target time-based attribute associated with the subject digital item is equal to or greater than the current threshold value specified for the respective minimum time-based threshold, the proposed set of parameters generated by the ensemble of instance assessment machine learning models specifies a new threshold value for the respective minimum time-based threshold, and the adapted subscriber-specific instance is configured to automatically route the subject digital item to the disposal queue when the target time-based attribute associated with the subject digital item is equal to or greater than the new threshold value specified for the respective minimum time-based threshold, wherein the new threshold value is greater than or less than the current threshold value specified for the respective minimum time-based threshold.

In one embodiment, the one or more corpora of subscriber-specific parameters specifies a current threshold value for a respective maximum threshold, the target subscriber-specific instance is configured to automatically route a subject digital item to a disposal queue when a target attribute associated with the subject digital item is less than the current threshold value specified for the respective maximum threshold, the proposed set of parameters generated by the ensemble of instance assessment machine learning models specifies a new threshold value for the respective maximum threshold, and the adapted subscriber-specific instance is configured to automatically route the subject digital item to the disposal queue when the target attribute associated with the subject digital item is less than the new threshold value specified for the respective maximum threshold, wherein the new threshold value is greater than or less than the current threshold value specified for the respective maximum threshold.

In one embodiment, the one or more corpora of subscriber-specific parameters specifies a current threshold value for a respective minimum machine learning model prediction threshold, the target subscriber-specific instance is configured to automatically predict, using a first set of machine learning models, a probability of a subject digital item having an adverse decision being reversed, the target subscriber-specific instance is configured to automatically correct one or more defects in the subject digital item to resolve the adverse decision associated with the subject digital item when the probability is greater than or equal to the current threshold value for the respective minimum machine learning model prediction threshold, the proposed set of parameters generated by the ensemble of instance assessment machine learning models specifies a new threshold value for the respective minimum machine learning model prediction threshold, and the adapted subscriber-specific instance is configured to automatically correct the one or more defects in the subject digital item to resolve the adverse decision when the probability is greater than or equal to the new threshold value specified for the respective minimum machine learning model prediction threshold, wherein the new threshold value is greater than or less than the current threshold value specified for the respective minimum machine learning model prediction threshold.

In one embodiment, the one or more corpora of subscriber-specific parameters specifies: a first computer-executable medical coding instruction that specifies a first set of medical events that require medical coding, and a second computer-executable medical coding instruction that specifies a second set of medical events that do not require medical coding, the target subscriber-specific instance is configured to automatically assess, by executing the first computer-executable medical coding instruction and the second computer-executable medical coding instruction, one or more medical events included in a subject digital item to determine whether the one or more medical events correspond to the first set of medical events or the second set of medical events, the target subscriber-specific instance automatically generates a digital artifact explaining the one or more medical events performed on a respective entity without any medical codes or coding-related information in response to the target subscriber-specific instance detecting that the one or more medical events corresponds to the second set of medical events, the proposed set of parameters generated by the ensemble of instance assessment machine learning models specifies a new iteration of the first computer-executable medical coding instruction and a new iteration of the second computer-executable medical coding instruction, the new iteration of the first computer-executable medical coding instruction includes one or more additional medical events that require medical coding when compared to the first set of medical events of the first computer-executable medical coding instruction, the new iteration of the second computer-executable medical coding instruction includes a reduced set of medical events that do not require medical coding when compared to the second set of medical events of the second computer-executable medical coding instruction, the adapted subscriber-specific instance is configured to automatically assess, by executing the new iteration of the first computer-executable medical coding instruction and the new iteration of the second computer-executable medical coding instruction, the one or more medical events included in the subject digital item to determine whether the one or more medical events correspond to the one or more additional medical events or the reduced set of medical events, and the adapted subscriber-specific instance automatically generates a corresponding digital artifact that explains the one or more medical events performed on the respective entity in association with one or more medical codes in response to the adapted subscriber-specific instance detecting that the one or more medical events corresponds to the one or more additional medical events.

In one embodiment, the one or more corpora of subscriber-specific parameters specifies a data acquisition computer procedure that the target subscriber-specific instance invokes to periodically fetch data from one or more electronic health record systems, the computer-implemented method further comprises detecting, using the ensemble of instance assessment machine learning models, an anomaly or misconfiguration in the data acquisition computer procedure, the proposed set of parameters generated by the ensemble of instance assessment machine learning models specifies a new data acquisition computer procedure that corrects the anomaly or misconfiguration detected in the data acquisition computer procedure, and the adapted subscriber-specific instance is configured to automatically invoke the new data acquisition computer procedure when performing subsequent data acquisition operations to retrieve new data from the one or more electronic health record systems during the target time span.

In one embodiment, the anomaly or misconfiguration detected in the data acquisition computer procedure includes at least a missing or incomplete parameter required for periodically fetching the data from the one or more electronic health record systems, and the new data acquisition computer procedure includes a parameter value for the missing or incomplete parameter.

In one embodiment, the one or more corpora of subscriber-specific parameters specifies a rule activation control value for a respective computer-executable rule, the rule activation control value is set to a first value in the one or more corpora of subscriber-specific parameters, the respective computer-executable rule is configured to automatically perform, using the one or more processors, an automated operation when a digital item satisfies one or more logical conditions included in the respective computer-executable rule, the respective computer-executable rule is deactivated in the target subscriber-specific instance based on the rule activation control value for the respective computer-executable rule being set to the first value in the one or more corpora of subscriber-specific parameters, the proposed set of parameters generated by the ensemble of instance assessment machine learning models recommends the rule activation control value for the respective computer-executable rule be set to a second value different from the first value, and the respective computer-executable rule is activated in the adapted subscriber-specific instance based on the rule activation control value for the respective computer-executable rule being set to the second value.

In one embodiment, the one or more corpora of subscriber-specific parameters specifies a rule activation control value for a respective computer-executable rule, the rule activation control value is set to a first binary value in the one or more corpora of subscriber-specific parameters, the respective computer-executable rule is configured to automatically perform, using the one or more processors, an automated operation when a digital item satisfies at least two logical conditions included in the respective computer-executable rule, the target subscriber-specific instance does not execute or use the respective computer-executable rule based on the rule activation control value for the respective computer-executable rule being set to the first binary value, the proposed set of parameters generated by the ensemble of instance assessment machine learning models specifies that the rule activation control value for the respective computer-executable rule is to be set to a second binary value different from the first binary value, and the adapted subscriber-specific instance executes or uses the respective computer-executable rule based on the rule activation control value for the respective computer-executable rule being set to the second binary value.

In one embodiment, the ensemble of instance assessment machine learning models generated the subset of the one or more corpora of subscriber-specific parameters before the target subscriber-specific instance was adapted to the adapted subscriber-specific instance, and the computer-implemented method further includes adapting an original subscriber-specific instance configured according to a default set of parameters defined by the multi-tenant cloud-based service to the target subscriber-specific instance configured according to the subset of the one or more corpora of subscriber-specific parameters, wherein adapting the original subscriber-specific instance to the target subscriber-specific instance includes modifying at least a first distinct automated workflow and a second distinct automated workflow encoded in the original subscriber-specific instance; generating an instance change log user interface object that includes: a first module identifier indicating that the first distinct automated workflow operates within a first distinct module of the multi-tenant cloud-based service, a first text-based description describing a function of the first distinct automated workflow, a first previous configuration state specifying a prior configuration state of the first distinct automated workflow, a first updated configuration state specifying a new configuration state of the first distinct automated workflow, a second module identifier indicating that the second distinct automated workflow operates within a second distinct module of the multi-tenant cloud-based service, a second text-based description describing a function of the second distinct automated workflow, a second previous configuration state specifying a prior configuration state of the second distinct automated workflow, and a second updated configuration state specifying a new configuration state of the second distinct automated workflow; and displaying, within the graphical user interface, the instance change log user interface object in association with the proposed set of parameters generated by the ensemble of instance assessment machine learning models and the simulation findings outputted by the one or more computer-based simulations.

In one embodiment, the instance change log user interface object displayed on the graphical user interface further includes: a first user identifier indicating an identity of a first user account that authorized the modification of the first distinct automated workflow from the first previous configuration state to the first updated configuration state, a first clock time indicating a time at which the first distinct automated workflow was modified from the first previous configuration state to the first updated configuration state, a second user identifier indicating that the first user account authorized the modification of the second distinct automated workflow from the second previous configuration state to the second updated configuration state, and a second clock time indicating a time at which the second distinct automated workflow was modified from the second previous configuration state to the second updated configuration state.

In one embodiment, the instance change log user interface object displayed on the graphical user interface further includes a user identifier identifying an artificial intelligence (AI) agent as a user that (i) authorized the modification of the first distinct automated workflow from the first previous configuration state to the first updated configuration state and (ii) authorized the modification of the second distinct automated workflow from the second previous configuration state to the second updated configuration state.

In one embodiment, the proposed set of parameters generated by the ensemble of instance assessment machine learning models includes a first individual parameter recommendation, a second individual parameter recommendation, and a third individual parameter recommendation, the first individual parameter recommendation specifies a first distinct proposed modification to a first setting of the target subscriber-specific instance, the second individual parameter recommendation specifies a second distinct proposed modification to a second setting of the target subscriber-specific instance, the third individual parameter recommendation specifies a third distinct proposed modification to a third setting of the target subscriber-specific instance, the graphical user interface displays the first individual parameter recommendation in association with a first approve selectable user interface element and a first reject selectable user interface element, the graphical user interface displays the second individual parameter recommendation in association with a second approve selectable user interface element and a second reject selectable user interface element, and the graphical user interface displays the third individual parameter recommendation in association with a third approve selectable user interface element and a third reject selectable user interface element.

In one embodiment, the computer-implemented method further includes receiving, via the graphical user interface, a first additional user input from the user selecting the first approve selectable user interface element, wherein selecting the first approve selectable user interface element indicates approval of the first individual parameter recommendation; receiving, via the graphical user interface, a second additional user input from the user selecting the second approve selectable user interface element, wherein selecting the second approve selectable user interface element indicates approval of the second individual parameter recommendation; receiving, via the graphical user interface, a third additional user input from the user selecting the third reject selectable user interface element, wherein selecting the third reject selectable user interface element indicates rejection of the third individual parameter recommendation, wherein: the user-modified version of the proposed set of parameters includes the first individual parameter recommendation and the second individual parameter recommendation based on receiving the first additional user input and the second additional user input, the user-modified version of the proposed set of parameters excludes the third individual parameter recommendation based on receiving the third additional user input, and the adapted subscriber-specific instance is configured according to the user-modified version of the proposed set of parameters based on detecting the user rejected the third individual parameter recommendation.

In one embodiment, the first setting is set to a first setting value within the target subscriber-specific instance, the second setting is set to a second setting value within the target subscriber-specific instance, the third setting is set to a third setting value within the target subscriber-specific instance, the first distinct proposed modification of the first individual parameter recommendation specifies a first proposed setting value for the first setting that is different from the first setting value, the second distinct proposed modification of the second individual parameter recommendation specifies a second proposed setting value for the second setting that is different from the second setting value, the third distinct proposed modification of the third individual parameter recommendation specifies a third proposed setting value for the third setting that is different from the third setting value, and configuring the adapted subscriber-specific instance according to the user-modified version of the proposed set of parameters includes setting: (i) the first setting of the adapted subscriber-specific instance to the first proposed setting value, (ii) the second setting of the adapted subscriber-specific instance to the second proposed setting value, and (ii) the third setting of the adapted subscriber-specific instance to the third setting value instead of the third proposed setting value.

In one embodiment, the ensemble of instance assessment machine learning models generates the proposed set of parameters based on assessing the plurality of distinct model input data objects against: (i) historical claim and payment data associated with the target subscriber-specific instance of the multi-tenant cloud-based service, (ii) a plurality of distinct payer contract data objects that each specify one or more payer-specific rules, one or more payer-specific reimbursement policies, and one or more payer-specific compliance requirements applicable to digital items processed by the target subscriber-specific instance, (iii) machine-readable industry standard specification data obtained from one or more external data sources, (iv) one or more machine-readable industry standard rule sets obtained from the one or more external data sources, and (v) historical benchmark data of a subscribing entity that corresponds to the target subscriber-specific instance.

In one embodiment, the target subscriber-specific instance corresponds to a subscribing entity subscribing to the multi-tenant cloud-based service, and the computer-implemented method further includes detecting, using the one or more processors, that a difference between an efficacy metric value computed for a subject efficacy metric during the target time span and a benchmark efficacy value defined for the subject efficacy metric exceeds a predefined deviation threshold, in response to detecting that the difference exceeds the predefined deviation threshold, generating, in real-time, an alert indicating the adapted subscriber-specific instance is behaving anomalously with respect to the subject efficacy metric, and transmitting, in real-time over a computer network, the alert to the subscribing entity.

In one embodiment, the computer-implemented method further includes executing an additional computer-based simulation simulating operation of the target subscriber-specific instance configured according to the subset of the one or more corpora of subscriber-specific parameters, wherein executing the additional computer-based simulation includes: providing a test set of digital items to the target subscriber-specific instance configured according to the subset of the one or more corpora of subscriber-specific parameters, and computing a first corpus of simulation findings based on processing the test set of digital items using the target subscriber-specific instance configured according to the subset of the one or more corpora of subscriber-specific parameters.

In one embodiment, executing the one or more computer-based simulations simulating the operation of the target subscriber-specific instance configured according to the proposed set of parameters includes: providing the test set of digital items to the target subscriber-specific instance configured according to the proposed set of parameters, and computing a second corpus of simulation findings based on processing the test set of digital items using the target subscriber-specific instance configured according to the proposed set of parameters, wherein the simulation findings outputted by the one or more computer-based simulations includes the second corpus of simulation findings.

In one embodiment, the computer-implemented method further includes generating, using the first corpus of simulation findings and the second corpus of simulation findings, a respective parameter change impact artifact for each parameter included in the proposed set of parameters, wherein: the respective parameter change impact artifact generated for a respective parameter of the proposed set of parameters graphically or textually explains a performance disparity resulting from processing the test set of digital items using (i) an original parameter value defined by the subset of the one or more corpora of subscriber-specific parameters for the respective parameter and (ii) a modified parameter value defined by the proposed set of parameters for the respective parameter, and the graphical user interface displays the respective parameter change impact artifact generated for each parameter included in the proposed set of parameters.

In one embodiment, the proposed set of parameters generated by the ensemble of instance assessment machine learning models includes: a first proposed value for a first proposed parameter, a second proposed value for a second proposed parameter, and a third proposed value for a third proposed parameter, displaying the proposed set of parameters on the graphical user interface includes: displaying, using a first distinct interactive user interface object of the graphical user interface, the first proposed value for the first proposed parameter, displaying, using a second distinct interactive user interface object of the graphical user interface, the second proposed value for the second proposed parameter, and displaying, using a third distinct interactive user interface object of the graphical user interface, the third proposed value for the third proposed parameter, and the computer-implemented method further comprises assessing, by the one or more processors, whether the user modified (i) the first proposed value using the first distinct interactive user interface object, (ii) the second proposed value using the second distinct interactive user interface object, and (iii) the third proposed value displayed using the third distinct interactive user interface object; detecting, by the one or more processors, the user did not modify (i-a) the first proposed value using the first distinct interactive user interface object, (ii-a) the second proposed value using the second distinct interactive user interface object, and (iii-a) the third proposed value using the third distinct interactive user interface object, wherein: the adapted subscriber-specific instance is configured according to the proposed set of parameters in response to detecting the user did not modify the first proposed value, the second proposed value, and the third proposed value.

In one embodiment, the proposed set of parameters generated by the ensemble of instance assessment machine learning models includes a first proposed value for a first proposed parameter a second proposed value for a second proposed parameter, and a third proposed value for a third proposed parameter, displaying the proposed set of parameters on the graphical user interface includes at least displaying, using a first distinct interactive user interface object of the graphical user interface, the first proposed value for the first proposed parameter, displaying, using a second distinct interactive user interface object of the graphical user interface, the second proposed value for the second proposed parameter, displaying, using a third distinct interactive user interface object of the graphical user interface, the third proposed value for the third proposed parameter, and the computer-implemented method further comprises: receiving, from the user, a first additional user input modifying the first proposed value to a first user-modified parameter value using the first distinct interactive user interface object; generating the user-modified version of the proposed set of parameters by: attributing the first user-modified parameter value to the first proposed parameter in response receiving the first additional user input, attributing the second proposed value to the second proposed parameter based on detecting that the user did not modify the second proposed value, and attributing the third proposed value to the third proposed parameter based on detecting that the user did not modify the third proposed value, wherein: the adapted subscriber-specific instance is configured according to the user-modified version of the proposed set of parameters based on detecting the user modified the first proposed value to the first user-modified parameter value.

### BRIEF DESCRIPTION OF THE FIGURES

FIG. 1 illustrates a schematic representation of a system 100 in accordance with one or more embodiments of the present application;

FIG. 2 illustrates an example method 200 in accordance with one or more embodiments of the present application;

FIG. 3 illustrates an example schematic for generating a proposed set of platform tuning parameters for a target subscriber and simulating the proposed set of platform tuning parameters in accordance with one or more embodiments of the present application;

FIG. 4 illustrates an example schematic for surfacing a proposed set of platform tuning parameters and associated simulation findings using a graphical user interface in accordance with one or more embodiments of the present application; and

FIGS. 5 - 25 illustrate example graphical user interfaces in accordance with one or more embodiments of the present application.

### DESCRIPTION OF THE PREFERRED EMBODIMENTS

The following description of the preferred embodiments of the invention is not intended to limit the invention to these preferred embodiments, but rather to enable any person skilled in the art to make and use this invention.

### System for Clinical Note Data Classification and Machine Learning Inference(s)-Informed Automated Routing of Electronic Communications

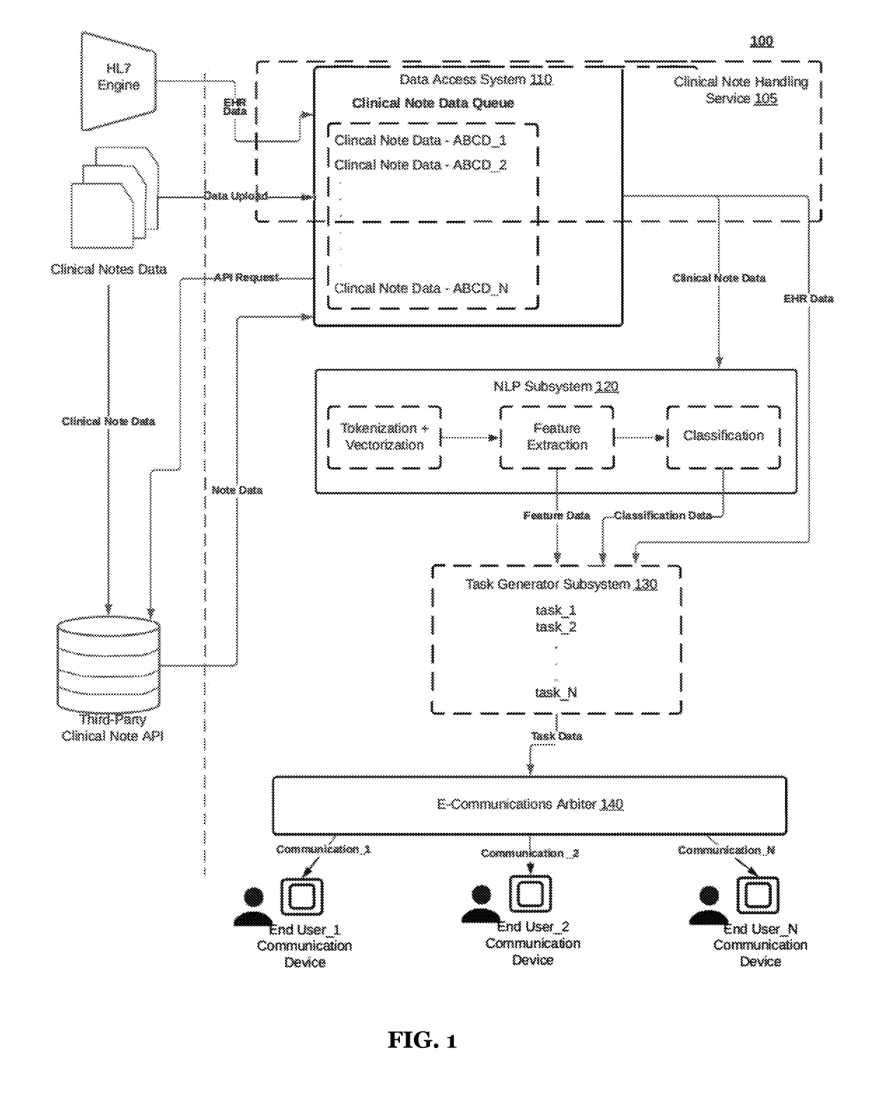

As shown in FIG. 1, a system 100 that implements clinical note data classification and uses machine learning inferences to inform an automated routing of electronic communications includes a clinical note data access and intake subsystem 110, feature extraction and classification subsystem 120, automated task generation subsystem 130, and an electronic communications subsystem 140.

### Clinical Note Data Handling and Automated Electronic Communications Service

The clinical note data handling and automated electronic communications service 105 implementing the system 100, sometimes referred to herein as the “clinical note handling service 105” may be implemented by a distributed network of computers (e.g., hosted on the cloud, etc.) and may be in operable and control communication with each of the subsystems of the system 100 and/or third-party subsystems and services. That is, the clinical note handling service 105 may include a centralized controlling computer server(s) and associated computing systems that encourages and/or controls the intelligent and accelerated clinical note data handling, clinical note data classification, and clinical note data-informed communications routing operations of each of the subsystems, described herein, (e.g., subsystems 110-140).

### Clinical Note Data Access + Intake Subsystem

The clinical note data access and intake subsystem 110, which may be sometimes referred to herein as the “data access system” 110, preferably functions to enable one or more electronic connections between the system 100 and one or more external systems of one or more subscribers to the clinical note handling service 105. The data access subsystem 110 may include one or more access modules that may function to establish or create content communication channels, which are sometimes referred to as “data handling nexus,” between the system 100 and systems associated with subscribers to the service 105. In one or more embodiments, the data handling nexus may include any suitable medium and/or method of transmitting digital items between at least two devices including, but not limited to, a service bus, a digital communication channel or line, and/or the like.

Additionally, or alternatively, the clinical note data access and intake subsystem 110 may provide a web-based graphical user interface or web application that may enable one or more subscribers to upload clinical note data (e.g., clinical note CSV files, and/or the like) directly into the system 100. Updating clinical note data via a CSV file and/or the like may enable system 100 to ingest historical clinical note data, thereby allowing system 100 to identify recommendations given prior to a live implementation of system 100.

In one or more embodiments, based on accessing or receiving clinical note data, the data access system 110 may function to store the clinical note data in a queue and preferably generate and/or associate identifying metadata including, but not limited to, a session identifier providing a unique identification value for a clinical session associated with a target clinical note, a patient identifier, a doctor identifier, a clinical note identifier, and/or the like. In such embodiments, the identifying metadata may be passed along with the clinical note data to one or more downstream subsystems (e.g., subsystem 120, subsystem 130, subsystem 140) to enable processing, tracking, account identification, and/or the like.

In one or more embodiments, the clinical note data handling service 105 may function to implement a clinical note data handling application programming interface (API) that enables programmatic communication, access, and control between the system 100 and the one or more sub-services within the system 100 and one or more (third-party) APIs associated with one or more subscribers to the clinical note data handling service 105.

Additionally, or alternatively, the data access system 110 may receive the clinical notes data via a health level seven (HL7) interface. In such embodiments, an electronic health record (EHR) system associated with a subscriber may periodically or in real-time send one or more HL7 messages comprising clinical note data to the data access system 110. In turn, the data access system 110 may receive the one or more HL7 messages via a secure channel (e.g., port) of the clinical note handling service 105 and provide the one or more HL7 messages to the NLP subsystem 120.

### NLP: Feature Identification + Extraction and Classification Subsystem

The feature extraction and classification subsystem 120, which may sometimes be referred to herein as a “NLP subsystem,” preferably functions to perform various natural language processing tasks including extracting features from clinical note data and computing one or more classification inferences and/or labels for each clinical note file being handled by the clinical note data handling service 105. The NLP subsystem 120 may additionally include one or more text processing modules and/or machine learning models that may tokenize textual data within a clinical note and vectorize and/or generate embeddings for each set of tokens and further cluster the tokens into semantically-related token groups or the like.

In one or more embodiments, the NLP subsystem 120 includes a machine learning module or subsystem that may be intelligently configured to predict various classifications for each clinical note document including, but not limited to, identifying whether a clinical note has a clinical recommendation, a number of clinical recommendations in a given clinical note, a type of clinical recommendation, a strength of a clinical recommendation, an urgency of a clinical recommendation, and/or the like. In such embodiments, the NLP subsystem 120 may include a plurality of distinct machine learning-based classification submodules, which may be outlined herein below in the method 200.

Additionally, or alternatively, in some embodiments, the NLP subsystem 120 may include extensible feature extraction and classification heuristics that may be applied alone or in combination with one or more machine learning-based classifiers described herein.

Additionally, or alternatively, the NLP subsystem 120 may implement one or more ensembles of pre-trained or trained machine learning models. The one or more ensembles of machine learning models may employ any suitable machine learning including one or more of: supervised learning (e.g., using logistic regression, using back propagation neural networks, using random forests, decision trees, etc.), unsupervised learning (e.g., using an Apriori algorithm, using K-means clustering), semi-supervised learning, reinforcement learning (e.g., using a Q-learning algorithm, using temporal difference learning), adversarial learning, and any other suitable learning style. Each module of the plurality can implement any one or more of: a regression algorithm (e.g., ordinary least squares, logistic regression, stepwise regression, multivariate adaptive regression splines, locally estimated scatterplot smoothing, etc.), an instance-based method (e.g., k-nearest neighbor, learning vector quantization, self-organizing map, etc.), a regularization method (e.g., ridge regression, least absolute shrinkage and selection operator, elastic net, etc.), a decision tree learning method (e.g., classification and regression tree, iterative dichotomiser 3, C4.5, chi-squared automatic interaction detection, decision stump, random forest, multivariate adaptive regression splines, gradient boosting machines, etc.), a Bayesian method (e.g., naïve Bayes, averaged one-dependence estimators, Bayesian belief network, etc.), a kernel method (e.g., a support vector machine, a radial basis function, a linear discriminate analysis, etc.), a clustering method (e.g., k-means clustering, density-based spatial clustering of applications with noise (DBSCAN), expectation maximization, etc.), a bidirectional encoder representation form transformers (BERT) for masked language model tasks and next sentence prediction tasks and the like, variations of BERT (i.e., ULMFiT, XLM UDify, MT-DNN, SpanBERT, RoBERTa, XLNet, ERNIE, KnowBERT, VideoBERT, ERNIE BERT-wwm, MobileBERT, TinyBERT, GPT, GPT-2, GPT-3, GPT-4 (and all subsequent iterations), ELMo, content2Vec, and the like), an associated rule learning algorithm (e.g., an Apriori algorithm, an Eclat algorithm, etc.), an artificial neural network model (e.g., a Perceptron method, a back-propagation method, a Hopfield network method, a self-organizing map method, a learning vector quantization method, etc.), a deep learning algorithm (e.g., a restricted Boltzmann machine, a deep belief network method, a convolution network method, a stacked auto-encoder method, etc.), a dimensionality reduction method (e.g., principal component analysis, partial least squares regression, Sammon mapping, multidimensional scaling, projection pursuit, etc.), an ensemble method (e.g., boosting, bootstrapped aggregation, AdaBoost, stacked generalization, gradient boosting machine method, random forest method, etc.), and any suitable form of machine learning algorithm. Each processing portion of the system 100 can additionally or alternatively leverage: a probabilistic module, heuristic module, deterministic module, or any other suitable module leveraging any other suitable computation method, machine learning method or combination thereof. However, any suitable machine learning approach can otherwise be incorporated in the system 100. Further, any suitable model (e.g., machine learning, non-machine learning, etc.) may be implemented in the various systems and/or methods described herein.

### Automated Recommendation Task Generator

The automated recommendation handling task and instructions generator 130, which may be sometimes referred to herein as a “tasks generator” 130 or “automated task generation subsystem” 130, preferably functions to automatically generate a clinical recommendation registry including one or more tasks and/or one or more instructions for handling and/or disposing of clinical recommendations identified within a clinical note. In one or more embodiments, the task generator 130 may take in as input a set of extracted features and a set of classification inferences computed by the NLP subsystem 120 to compose and/or structure a given registry. It shall be noted that, in some portions of the disclosure, a “clinical recommendation registry” may be referred to as a “clinical recommendation worklist” or the like.

A given clinical recommendation registry preferably includes an enumeration of tasks and/or computer-executable instructions that may be automatically executed by the clinical note handling service 105. Additionally, or alternatively, the clinical recommendation registry may include patient session identifier (ID) data, clinical recommendation ID data, patient communications account data (e.g., email, phone number, messaging ID, etc.) that may be used as input in structuring one or more electronic communications to a given patient, as described herein and using at least e-communications arbiter 140.

### Automated E-Communications Arbiter & Routing

The electronic communications subsystem 140, which may be sometimes referred to herein as an “e-communications arbiter” 140, preferably functions to take in as input a clinical recommendation registry associated with a target clinical recommendation and structure, as output, an automated electronic communication scheme for handling and/or disposing of the target clinical recommendation. Accordingly, the e-communications arbiter 140 may function to intelligently select an optimal communication channel for communicating with an end user or patient, structuring communication parameters, such as a communication schedule and/or communication frequency and composing message content for each communication to the end user. In one or more embodiments, the e-communication arbiter may function to employ a selection matrix or the like for selecting a most optimal communication channel and may further employ pre-trained language models and/or messaging templates to compose messaging content for a given communication.

### Method for Machine Learning-Based Tuning of Platform Parameters and Settings within Subscriber-Specific Instances of a Digital Platform

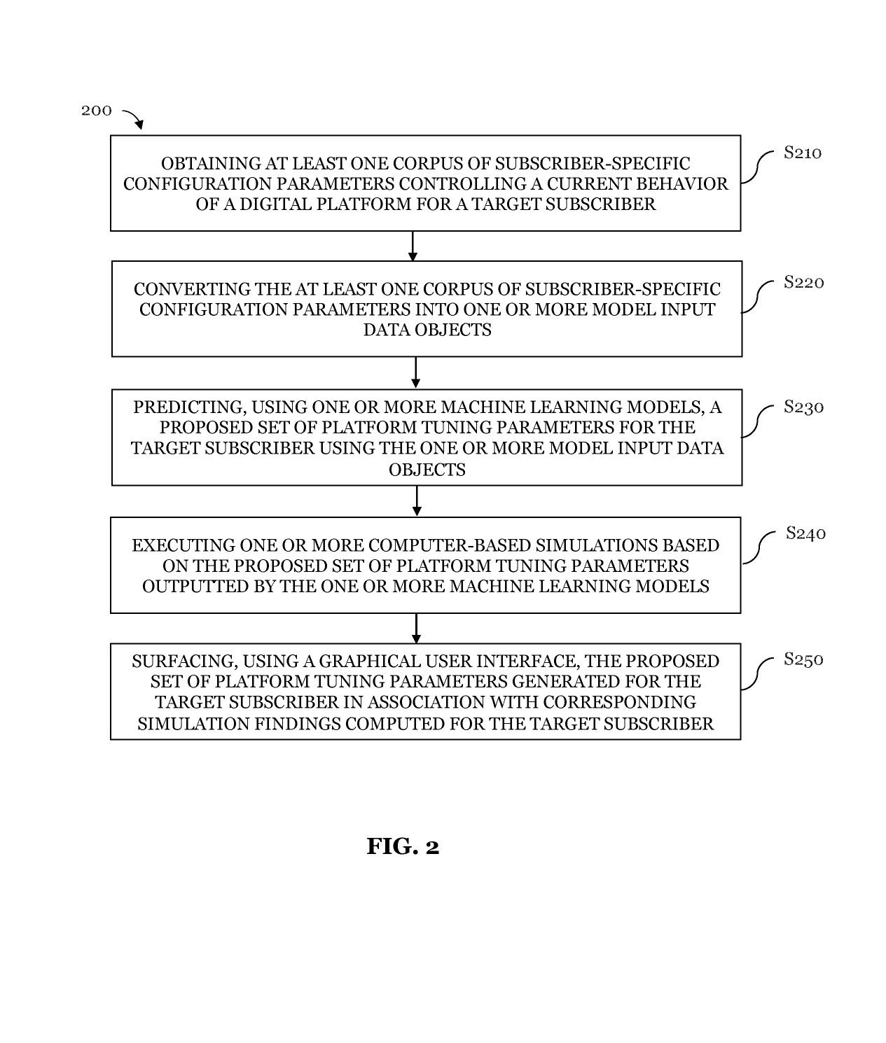

As shown in FIG. 2, the method 200 for machine learning-based tuning of platform parameters and settings within subscriber-specific instances of a digital platform (e.g., multi-tenant cloud-based service or the like) includes obtaining at least one corpus of subscriber-specific configuration parameters controlling a current behavior of a digital platform for a target subscriber S210, converting the at least one corpus of subscriber-specific configuration parameters into one or more model input data objects S220, predicting, using one or more machine learning models, a proposed set of platform tuning parameters for the target subscriber using the one or more model input data objects S230, executing one or more computer-based simulations based on the proposed set of platform tuning parameters outputted by the one or more machine learning models S240, and surfacing, using a graphical user interface, the proposed set of platform tuning parameters generated for the target subscriber in association with corresponding simulation findings computed for the target subscriber S250.

The systems, methods, and embodiments described herein may be implemented in a multi-tenant, cloud-based computing service in which a service provider hosts a plurality of subscriber-specific instances of the multi-tenant, cloud-based computing service. Each subscriber-specific instance may execute one or more automated workflows, rules, and machine-controlled operations that are configured according to subscriber-specific parameters that govern a runtime configuration and/or runtime behavior of the respective subscriber-specific instance. For instance, in a non-limiting example, a first subscriber-specific instance may correspond to a first subscriber and be configured according to a first set of subscriber-specific parameters defined by the first subscriber, while a second subscriber-specific instance may correspond to a second subscriber and be configured according to a second set of subscriber-specific parameters defined by the second subscriber. It shall be recognized that, in such a non-limiting example, the first set of subscriber-specific parameters and the second set of subscriber-specific parameters may differ in at least one parameter that controls a runtime configuration or runtime behavior of the respective subscriber-specific instance, such that the first subscriber-specific instance and the second subscriber-specific instance execute different automated workflows, apply different threshold-based routing logic, or invoke different machine-controlled operations during runtime.

In conventional multi-tenant cloud-based services, subscriber-specific instances become stale, misconfigured, or sub-optimally tuned over time as subscriber requirements, operational policies, and data characteristics evolve. As described in more detail herein, in one or more embodiments, the systems, methods, and embodiments described herein may function to automatically detect when a subscriber-specific instance has become stale (e.g., has not been tuned during a predetermined time span), automatically assess, in real-time, one or more corpora of subscriber-specific parameters characterizing a runtime behavior of the subscriber-specific instance, and automatically adapt, in real-time, a runtime configuration of the subscriber-specific instance based on the assessment.

Automatically detecting stale subscriber-specific instances and adapting their respective runtime configurations as described herein improves computing performance of the multi-tenant cloud-based computing service by reducing unnecessary computational work performed by the subscriber-specific instances. By tuning subscriber-specific parameters that control automated workflows, rule execution, threshold-based routing logic, and machine-controlled operations, the systems, methods, and embodiments reduce repeated execution of inapplicable workflows, reduce and/or prevent invocation of machine learning models on low-value digital items, and prevent redundant processing paths, thereby reducing central processing unit (CPU) utilization, lowering memory consumption associated with transient data objects and workflow buffering, and decreasing processing latency. As a result, subscriber-specific instances execute digital operations more efficiently, with improved throughput and more effective utilization of shared computing resources within the multi-tenant cloud-based computing service. Stated another way, the systems, methods, and embodiments described herein improve computing performance of a subscriber-specific instance by modifying runtime behavior of the subscriber-specific instance in a manner that reduces unnecessary computational operations, lowers processor and memory utilization, and improves execution efficiency of automated digital operations (e.g., workflows, etc.).

Additionally, in one or more embodiments, the systems, methods, and computer-program products described herein provide improvements over conventional graphical user interfaces by enabling a single user input selecting a tune instance control button displayed on a target graphical user interface to simultaneously implement or encode runtime configuration changes across a plurality of distinct modules of the subscriber-specific instance in a single, unified operation. In this way subscribers do not need to locate or navigate to individual configuration settings via multiple graphical user interfaces, thereby requiring fewer user inputs to adapt the runtime configuration of the subscriber-specific instance and reducing processor usage associated with repeated interface navigation and configuration operations. Stated another way, the graphical user interface described herein enables automated modification of multiple runtime configuration settings through a single user interaction, such that subscribers may adapt runtime behavior of the subscriber-specific instance without requiring knowledge of how or where individual configuration settings are implemented, encoded, or stored across the plurality of modules, which would otherwise require interaction with or navigation across multiple graphical user interfaces. Stated differently, the graphical user interface described herein enables automated and simultaneous modification of multiple runtime configuration settings through a single user interaction, thereby allowing subscribers to adapt runtime behavior of the subscriber-specific instance without requiring knowledge of how or where individual configuration settings are implemented across the plurality of modules and/or navigation across multiple graphical user interfaces.

It shall be recognized that, in one or more embodiments, the configuration of a subscriber-specific instance is adapted in real time or near real time while the subscriber-specific instance remains active, such that the subscriber-specific instance continues to process digital items during the adaptation process, thereby dynamically reconfiguring system behavior without redeployment, interruption of service, or suspension of automated workflows. At least one technical advantage of such an adaptation is that runtime configuration changes may be applied without halting processing pipelines or draining in-flight digital items, thereby reducing downtime, preserving processing throughput, and maintaining efficient utilization of computing resources. In other words, at least one technical advantage of such an adaptation is that configuration changes are implemented without pausing execution of automated workflows, thereby maintaining processing continuity and system throughput. Stated another way, at least one technical advantage of such an adaptation is that configuration changes are applied while processing continues, thereby avoiding resource underutilization and performance degradation associated with service restarts.

It shall be further recognized that, in one or more embodiments, adapting runtime configurations as described herein may improve computing performance of subscriber-specific instances by reducing inefficient execution paths (e.g., preventing execution of target automated workflows, combining computer-executable rules, etc.) and compute resource usage. In some implementations, such improvements may correspond to reductions in processing time or resource consumption on the order of approximately 10%, 20%, and in certain scenarios up to about 50%, depending on the initial configuration state of the subscriber-specific instance and characteristics of the processed digital items.

### Obtaining Subscriber-Specific Platform Configuration Parameters

S210, which includes obtaining subscriber-specific configuration parameters, may function to obtain, from computer memory, at least one corpus of subscriber-specific configuration parameters controlling a current behavior of a digital platform. A corpus of subscriber-specific configuration parameters, as generally referred to herein, may include one or more platform settings specific to a target subscriber that govern execution and/or operation of one or more modules and/or one or more submodules of the digital platform. It shall be recognized that the phrase “corpus of subscriber-specific configuration parameters” may be interchangeably referred to herein as “subscriber-specific system settings,” “platform settings of a subscriber,” “subscriber-specific platform configuration parameters,” and/or the like.

In one or more embodiments, the at least one corpus of subscriber-specific configuration parameters may configure, encode, control, or otherwise influence operational states and automated operations of the digital platform. In such an embodiment, each subscriber to the digital platform may have an associated corpus of subscriber-specific configuration parameters stored in the computer memory. For instance, in a non-limiting example, a first corpus of subscriber-specific configuration parameters stored in the computer memory may correspond to a first distinct subscriber of the digital platform, a second corpus of subscriber-specific configuration parameters stored in the computer memory may correspond to a second distinct subscriber of the digital platform, and a third corpus of subscriber-specific configuration parameters stored in the computer memory may correspond to a third distinct subscriber of the digital platform. It shall be recognized that additional subscribers may likewise be associated with additional distinct corpora of subscriber-specific configuration parameters, and that no limitation is made as to the number of such corpora or subscribers that may be concurrently supported by the digital platform.

Accordingly, in one or more embodiments, operation of the digital platform (e.g., multi-tenant cloud-based service) may be individualized for a respective subscriber based in part on the corpus of subscriber-specific configuration parameters corresponding to the respective subscriber. For instance, with continued reference to the above non-limiting example, the digital platform may operate according to the first corpus of subscriber-specific configuration parameters for the first distinct subscriber, may operate according to the second corpus of subscriber-specific configuration parameters for the second distinct subscriber, and may operate according to the third corpus of subscriber-specific configuration parameters for the third distinct subscriber. In other words, the digital platform does not operate uniformly across all subscribers but instead operates differently for each subscriber based in part on the corresponding corpus of subscriber-specific configuration parameters.

In one or more embodiments, a system or service implementing method 200 may function to execute a computer-based scanning operation that automatically retrieves the at least one corpus of subscriber-specific configuration parameters associated with a target subscriber on a periodic basis. In other words, in such an embodiment, the system or service implementing method 200 may function to iteratively retrieve, from computer memory, the at least one corpus of subscriber-specific configuration parameters controlling a current operation or behavior of the digital platform for the target subscriber. For instance, in a non-limiting example, S210 may function to automatically obtain, using one or more processors, the corpus of subscriber-specific configuration parameters for the target subscriber on an hourly, daily, weekly, quarterly, or yearly basis. At least one technical benefit of periodically obtaining the corpus of subscriber-specific configuration parameters for the target subscriber enables the system or service implementing method 200 to automatically assess the corpus of subscriber-specific configuration parameters associated with the target subscriber, automatically propose platform tuning parameters for the target subscriber, and/or automatically implement the proposed platform tuning parameters to adapt operation of the digital platform for the target subscriber.

In one or more embodiments, the digital platform may include a plurality of distinct modules or submodules that collectively define an overall capability of the digital platform (e.g., multi-tenant cloud-based service or the like). Each module or submodule of the multi-tenant cloud-based service (e.g., digital platform or the like), in some embodiments, may be operably configured to enable a subscriber, a user or automated operator (e.g., artificial intelligence (AI) agent or the like) to perform a distinct set of tasks, functions, and/or operations. In such an embodiment, the digital platform may include a first distinct module that enables a respective subscriber or automated operator to perform a first distinct set of tasks, functions, and/or operations, a second distinct module that enables the respective subscriber or automated operator to perform a second distinct set of tasks, functions, and/or operations, a third distinct module that enables the respective subscriber or automated operator to perform a third distinct set of tasks, functions, and/or operations, and a fourth distinct module that enables the respective subscriber or automated operator to perform a fourth distinct set of tasks, functions, and/or operations. Stated another way, in one or more embodiments, each distinct module of the digital platform may be encoded with a distinct set of subscriber-specific parameters to automatically or semi-automatically perform a respective set of tasks, functions, and/or operations using predefined control logic, configurable data structures, computer-executable rule-based heuristics, and/or computer-executable rule-based workflows.

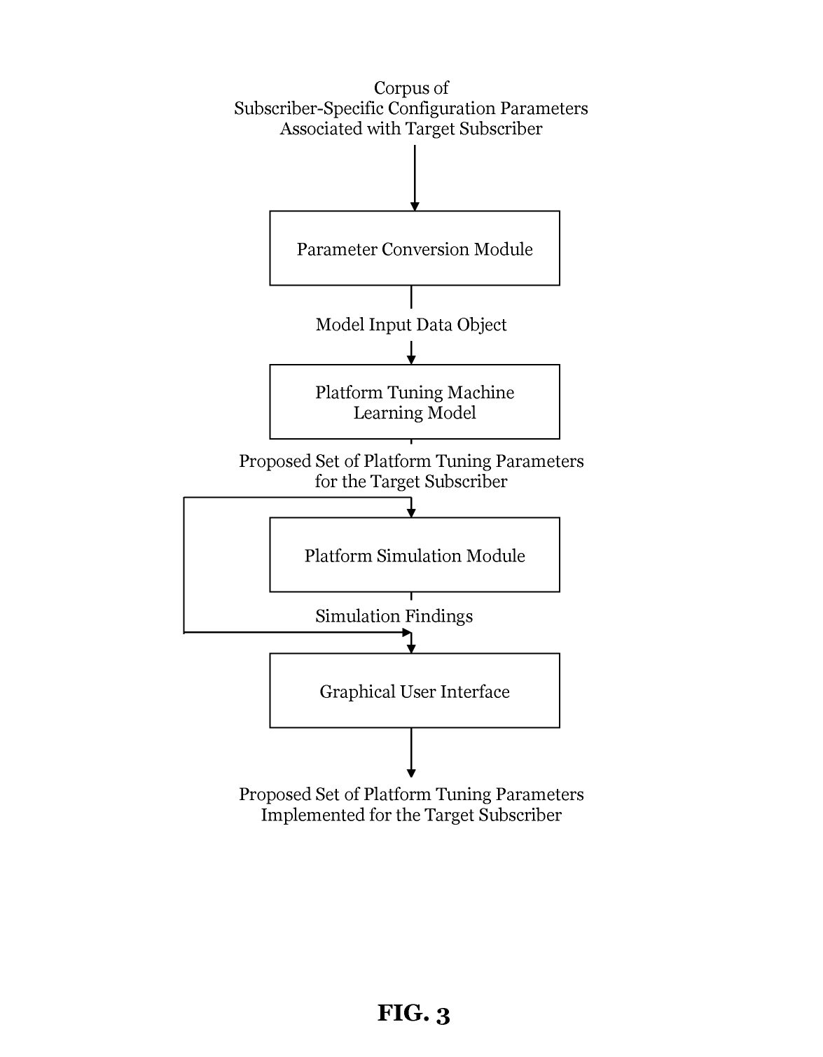

Accordingly, in one or more embodiments, S210 may function to obtain, from computer memory, a single data structure of a target subscriber (e.g., single dataset, single corpus of subscriber-specific configuration parameters, etc.) that includes the subscriber-specific configuration parameters for all modules (e.g., the first module, the second module, the third module, the fourth module, etc.) of the digital platform, as shown generally by way of example in FIG. 3. In a non-limiting example, the single data structure may include a current minimum timing-based threshold value that the first module uses to automatically transition a pending digital item (e.g., pending digital claim) to a closed, terminated, or dispositioned state upon satisfying the current minimum timing-based threshold value (e.g., transitioning a digital item in a pending state to a closed, terminated, or dispositioned state when the digital item remains in the pending state for at least thirty days (e.g., the current minimum timing-based threshold value)), a current maximum demand-based threshold value that the first module uses to automatically transition a pending digital item (e.g., pending digital claim) to a closed, terminated, or dispositioned state upon satisfying the current maximum demand-based threshold value (e.g., transitioning a digital item in a pending state to a closed, terminated, or dispositioned state when a metadata value of the digital item is less than the current maximum demand-based threshold value), and an efficacy metric value that represents a measure of performance of the first module.

Additionally, or alternatively, in such a non-limiting example, the single data structure may include a plurality of rule-based heuristics (e.g., computer-executable rules, etc.) used by the second module (e.g., at least two rule-based heuristics, at least ten rule-based heuristics, at least one hundred rule-based heuristics, at least one thousand rule-based heuristics, etc.) that specify conditional logic for processing digital items (e.g., digital claims). For instance, in a non-limiting example, the plurality of rule-based heuristics may collectively indicate which type of digital items (e.g., digital claims) require assignment (or attribution) of one or more coding labels of a predetermined set of coding labels and which type of digital items (e.g., digital claims) do not require such coding labels.

Additionally, or alternatively, in such a non-limiting, the single data structure may include a plurality of third-party transaction parameters (e.g., contract parameters, payer contract parameters, payer compliance rules, etc.) encoded within the third module of the digital platform. The plurality of third-party transaction parameters, in such a non-limiting example, may include a plurality of timing rules, a plurality of escalation conditions, a plurality of contractual terms, and/or a plurality of compliance handling rules that control how the digital platform manages, monitors, and interacts with one or more third-party entities (e.g., healthcare payers, etc.).

Additionally, or alternatively, in such a non-limiting example, the single data structure may include a plurality of data acquisition parameters that control how the fourth module collects, ingests, and/or normalizes data (e.g., claim data, historical claim data, payment data, historical payment data, etc.) from one or more external data sources and/or one or more internal data sources. For instance, in a non-limiting example, the plurality of data acquisition parameters may include one or more computer-executable data acquisition instructions that, when executed, obtains data from one or more third-party entities and/or from an electronic health record (EHR) system. In other words, the data acquisition parameters encode how and when the digital platform interfaces with external and internal data sources to retrieve, validate, and integrate subscriber-specific data for downstream processing within the digital platform.

Stated differently, in one or more embodiments, the single data structure may include a plurality of subscriber-specific platform settings, a plurality of subscriber-specific automated workflows, a plurality of subscriber-specific computer-executable instructions, and/or a plurality of subscriber-specific policies encoded within all modules and submodules of the digital platform for a respective subscriber, which collectively function to control execution, coordination, and operation of the digital platform for the respective subscriber. It shall be further recognized that, in one or more embodiments, the single data structure may further include a plurality of target performance metrics to be achieved by the digital platform (e.g., the subscriber-specific instance of the multi-tenant cloud-based service or the like) across all modules and submodules of the digital platform (e.g., the subscriber-specific instance of the multi-tenant cloud-based service or the like). A target performance metric, in some embodiments, may refer to a predefined target quantitative value that the respective subscriber expects the digital platform (e.g., the subscriber-specific instance of the multi-tenant cloud-based service or the like) to meet or exceed.

Alternatively, in one or more embodiments, obtaining the at least one corpus of subscriber-specific configuration parameters for a target subscriber may include retrieving, from the computer memory, a distinct corpus of subscriber-specific configuration parameters encoded within each module or submodule of the digital platform. In such an embodiment, the digital platform may function to store, within the computer memory, a distinct module configuration data structure for each distinct module of the digital platform. For instance, in a non-limiting example, the computer memory may store a first distinct module configuration data structure that specifies a plurality of subscriber-specific module settings, subscriber-specific workflows, subscriber-specific parameters, and/or subscriber-specific policies controlling operation of only a first module of the digital platform, a second distinct module configuration data structure that specifies a plurality of subscriber-specific module settings, subscriber-specific workflows, subscriber-specific parameters, and/or subscriber-specific policies controlling operation of only a second module of the digital platform, a third distinct module configuration data structure that specifies a plurality of subscriber-specific module settings, subscriber-specific workflows, subscriber-specific parameters, and/or subscriber-specific policies controlling operation of only a third module of the digital platform, a fourth distinct module configuration data structure that specifies a plurality of subscriber-specific module settings, subscriber-specific workflows, subscriber-specific parameters, and/or subscriber-specific policies controlling operation of only a fourth module of the digital platform, and so forth. Accordingly, in such an embodiment, when the system or service initiates a request to obtain the at least one corpus of subscriber-specific configuration parameters for a target subscriber, S210 may function to retrieve, from computer memory, each distinct module configuration data object associated with the target subscriber (e.g., the first distinct module configuration data structure, the second distinct module configuration data structure, the third distinct module configuration data structure, and the fourth distinct module configuration data structure, etc.).

It shall be further recognized that, in one or more embodiments, each distinct module configuration data structure may include a plurality of target performance metrics that the target subscriber expects the digital platform (e.g., the subscriber-specific instance of the multi-tenant cloud-based service or the like) to achieve for the corresponding module or submodule.

Additionally, or alternatively, in one or more embodiments, S210 may function to automatically detect, in real-time using one or more processors, a target subscriber-specific instance of a multi-tenant cloud-based service that has not been tuned or optimized during a predetermined time span, such as the last seven days. For instance, in a non-limiting example, S210 may detect that a target subscriber-specific instance of a plurality of distinct subscriber-specific instances has not had any runtime configuration parameters, settings, or the like modified, updated, or validated during the predetermined time span (e.g., last seven days, last 30 days, etc.) and, in response, designate the target subscriber-specific instance as a candidate for automated assessment and adaptation of its respective runtime configuration.

Accordingly, in one or more embodiments, in response to detecting that the target subscriber-specific instance has not been tuned or optimized during the predetermined time span, S210 may function to obtain, in real-time from one or more computer databases, one or more corpora of subscriber-specific parameters characterizing a runtime behavior of the target subscriber-specific instance. It shall be recognized that, in such an embodiment, at least a subset of the one or more corpora of subscriber-specific parameters may control a runtime of the target subscriber-specific instance. In other words, the subset of the one or more corpora of subscriber-specific parameters controlling the runtime of the target subscriber-specific instance may control how and when automated workflows encoded with the target subscriber-specific instance are executed, which computer-executable rules encoded with the target subscriber-specific instance are activated or deactivated, how digital items are routed through processing pipelines of the target subscriber-specific instance, execution order of automated workflows encoded with the target subscriber-specific instance, activation thresholds for computer-executable rules encoded with the target subscriber-specific instance, routing logic for digital items, and conditions under which machine-controlled operations are invoked during runtime of the target subscriber-specific instance.

### Converting the Subscriber-Specific Configuration Parameters into Model Input Data Objects

S220, which includes converting the at least one corpus of subscriber-specific configuration parameters, may function to convert the at least one corpus of subscriber-specific configuration parameters of a target subscriber into one or more model input data objects. A model input data object generated for a target subscriber, as generally referred to herein, may refer to a structured and/or normalized representation of subscriber-specific platform configuration parameters extracted from one or more modules or submodules of the digital platform that is in a format suitable for input into at least one downstream machine learning model (e.g., an ensemble of instance assessment machine learning models, a single instance assessment machine learning model, etc.).

In one or more embodiments, S220 may function to transform the at least one corpus of subscriber-specific configuration parameters of a target subscriber into one or more model input data objects that is compatible with an input schema of one or more downstream machine learning models (e.g., one or more instance assessment machine learning models or the like). At least one technical benefit of transforming the at least one corpus of subscriber-specific configuration parameters of the target subscriber into the one or more model input data objects enables the one or more downstream machine learning models to predict a proposed set of platform tuning parameters for the target subscriber based on assessing the one or more model input data objects and simulate a performance of the digital platform for the target subscriber using the proposed set of platform tuning parameters.

In a first implementation, S210 may function to obtain the single data structure associated with a target subscriber and, in turn, convert the single data structure into a single model input data object, as shown generally by way of example in FIG. 3. The single model input data structure, in such an implementation, may include a normalized representation of the subscriber-specific configuration parameters included in all modules and submodules of the digital platform (e.g., multi-tenant cloud-based service). Accordingly, in such an implementation, the single model input data structure may be provided as input to one or more downstream machine learning models that may predict a proposed set of platform tuning parameters for the target subscriber based on assessing the single model input data object and/or simulate a performance of the digital platform for the target subscriber using the proposed set of platform tuning parameters.

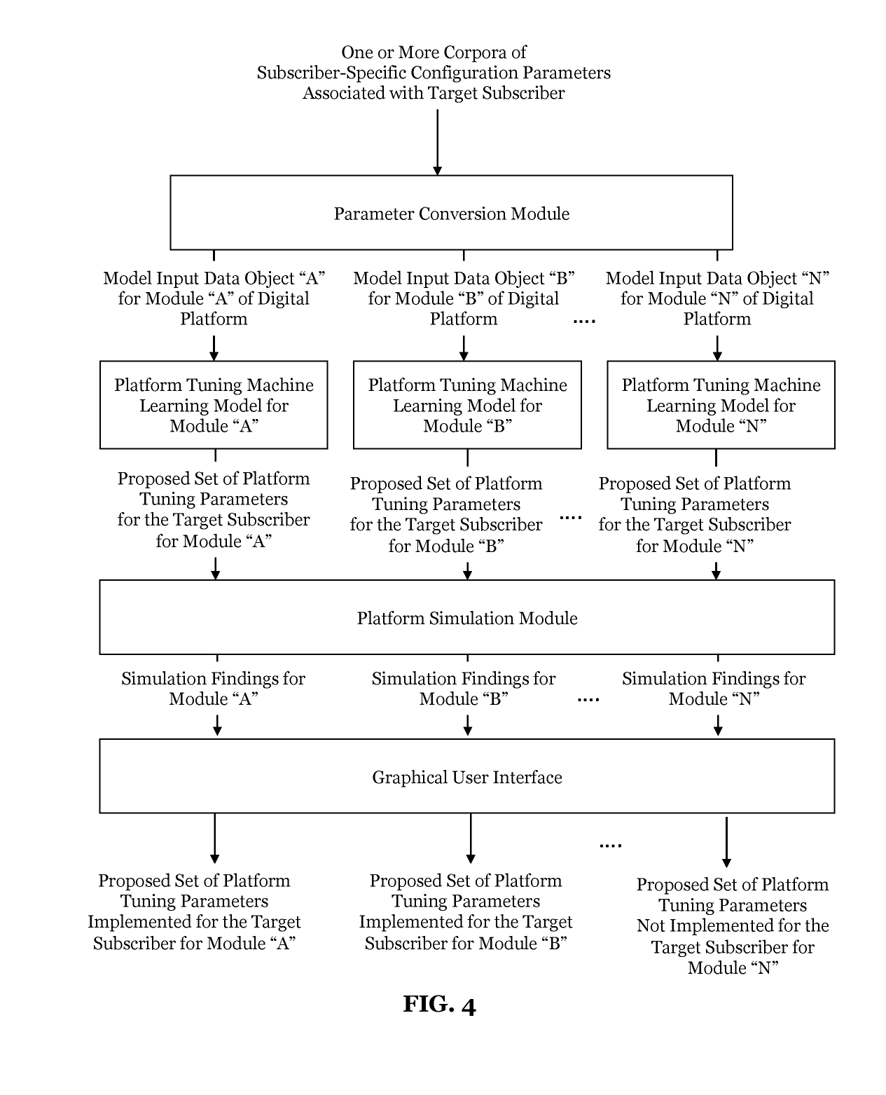

In a second implementation, S210 may function to obtain the single data structure associated with a target subscriber and, in turn, convert the single data structure into a plurality of distinct model input data objects, as shown generally by way of example in FIG. 4. In such an implementation, each distinct model input data object of the plurality of distinct model input data objects may include a structured and normalized subset of subscriber-specific configuration parameters corresponding to only one module or submodule of the digital platform. For instance, a first model input data object may only include subscriber-specific configuration parameters corresponding to the first module, a second model input data object may only include subscriber-specific configuration parameters corresponding to the second module, a third model input data object may only include subscriber-specific configuration parameters corresponding to the third module, and so forth.

In a third implementation, S210 may function to obtain a distinct corpus of subscriber-specific configuration parameters associated with each module or submodule of the digital platform. In such an implementation, S220 may function to convert each distinct corpus of subscriber-specific configuration parameters into a corresponding model input data object. For instance, in a non-limiting example, the first distinct corpus of (e.g., subscriber-specific) configuration parameters associated with the first module may be transformed into a first model input data object, the second distinct corpus of (e.g., subscriber-specific) configuration parameters associated with the second module may be transformed into a second model input data object, and so on. Accordingly, in such a non-limiting example, the plurality of model input data objects may collectively represent a module-level decomposition of the subscriber-specific configuration parameters, thereby enabling downstream machine learning models (e.g., downstream instance assessment machine learning models or the like) to selectively analyze, evaluate, or optimize the operation of individual modules or submodules of the digital platform (e.g., subscriber-specific instance of the multi-tenant cloud-based service or the like) with a higher degree of granularity and accuracy.

It shall be recognized that, in one or more embodiments, converting a subject corpus of subscriber-specific configuration parameters into one or more model input data objects may include executing one or more computer-based preprocessing operations that restructure, transform, or encode the subscriber-specific configuration parameters into a machine-readable format compatible with one or more downstream machine learning models (e.g., ensemble of instance assessment machine learning models or the like). For instance, in a non-limiting example, the conversion may include (i) extracting raw configuration values (e.g., threshold values, workflow rules, or contractual parameters) from the corpus of subscriber-specific configuration parameters, (ii) normalizing such values into a standardized numerical or categorical representation, (iii) mapping each normalized representation into predefined input feature vectors that align with the schema of the downstream machine learning models, and/or (iv) packaging the resulting feature vectors into one or more structured model input data objects. Stated another way, conversion may include translating heterogeneous and subscriber-specific configuration parameters into structured, machine-consumable representations such as vectors, tensors, or tabular feature sets that are compatible with supervised learning models, unsupervised learning models, reinforcement learning models, or any other suitable machine learning model implemented by the digital platform.

At least one technical benefit of converting the at least one corpus of subscriber-specific configuration parameters into one or more model input data objects as described herein includes improved parameter assessment, efficiency, and interpretability for the one or more downstream machine learning models. By translating heterogeneous subscriber-specific values (e.g., workflow rules, contractual terms, coding requirements, timing thresholds, data acquisition settings, etc.) into normalized model input data objects, the digital platform reduces variability and noise that might otherwise impede accurate machine learning-based parameter tuning predictions. Additionally, the conversion ensures that model inputs adhere to a standardized schema across all subscribers, thereby enabling downstream machine learning models to more efficiently learn generalized patterns, identify anomalies, or recommend platform tuning parameters with greater precision. Furthermore, conversion into structured model input data objects supports modular analysis at the level of individual modules or submodules of the digital platform, allowing machine learning models to selectively simulate, optimize, or evaluate subscriber-specific performance with finer granularity and scalability across a large number of subscribers.

Stated another way, in one or more embodiments, in response to obtaining the one or more corpora of subscriber-specific parameters, S220 may function to convert, in real-time or near real-time, the one or more corpora of subscriber-specific parameters into a plurality of distinct model input data objects operably configured for input into an ensemble of instance assessment machine learning models. An instance assessment machine learning model, in one or more embodiments, may be configured to process one or more of the plurality of distinct model input data objects to generate a proposed set of parameters for controlling a future runtime behavior of the target subscriber-specific instance.

In a first implementation, S210 may function to convert the one or more corpora of subscriber-specific parameters into the plurality of distinct model input data objects by grouping subscriber-specific parameters by type and/or runtime function. In such an implementation, all threshold-based subscriber-specific parameters may be grouped into or included in a first model input data object of the plurality of distinct model input data objects, all computer-executable instructions defining automated workflows or machine-controlled operations may be grouped into or included in a second model input data object of the plurality of distinct model input data objects, all data acquisition computer procedures may be grouped into or included in a third model input data object of the plurality of distinct model input data objects, and all rule activation control values governing execution of computer-executable rules, instructions, heuristics, or the like may be grouped into or included in a fourth model input data object of the plurality of distinct model input data objects.

Stated another way, in one or more embodiments, the first model input data object may only include threshold-based subscriber-specific parameters, such as a threshold category or type (e.g., minimum threshold, maximum threshold, time-based threshold, or probability-based threshold), one or more corresponding threshold values, and one or more identifiers or attributes indicating how and where the threshold is applied during runtime of the target subscriber-specific instance. Similarly, in one or more embodiments, the second model input data object may only include computer-executable instructions defining automated workflows, processing logic, or machine-controlled operations executed by the target subscriber-specific instance, such as workflow definitions, executable logic blocks, conditional execution paths, and associations between workflow steps that govern how digital items are processed during runtime. Similarly, in one or more embodiments, the third model input data object may only include data acquisition computer procedures associated with the target subscriber-specific instance, such as computer procedures defining how data is requested, retrieved, ingested, refreshed, or synchronized from one or more internal or external data sources, including one or more parameters specifying timing, frequency, source identifiers, authentication requirements, or data formatting rules. Similarly, in one or more embodiments, the fourth model input data object may only include rule activation control values associated with computer-executable rules, instructions, heuristics, or policies of the target subscriber-specific instance, such as binary or multi-state control values indicating whether a respective rule is enabled, disabled, conditionally executed, or otherwise selectively applied during runtime.

In a second implementation, S220 may function to convert the one or more corpora of subscriber-specific parameters into the plurality of distinct model input data objects by grouping subscriber-specific parameters included in the one or more corpora of subscriber-specific parameters on a per-module basis. In such an implementation, each model input data object of the plurality of distinct model input data objects may correspond to a respective distinct module of the target subscriber-specific instance and may include all subscriber-specific parameters that control runtime behavior of the respective distinct module. Stated another way, in one or more embodiments, a respective model input data object corresponding to a subject module may include all threshold-based parameters, computer-executable instructions, data acquisition computer procedures, rule activation control values, and other runtime configuration settings associated with the subject module. By encapsulating all module-specific runtime controls of a subject module within a single model input data object, the ensemble of instance assessment machine learning models may assess and generate proposed parameter changes for the subject module as a coherent unit, while preserving interdependencies between parameters that jointly govern execution behavior of the subject module during runtime of the target subscriber-specific instance.

### Predicting a Proposed Set of Platform Tuning Parameters for the Target Subscriber

S230, which includes predicting platform tuning parameters, may function to predict a proposed set of platform tuning parameters for a target subscriber using one or more machine learning models (e.g., the one or more downstream machine learning models, the ensemble of instance assessment machine learning models, etc.). A proposed set of platform tuning parameters or the like, as generally referred to herein, may refer to a collection of system-recommended values, conditions, or rules that are computationally derived and intended to guide, modify, or optimize operation of the digital platform for a target subscriber. It shall be recognized that the phrase “proposed set of platform tuning parameters” may be interchangeably referred to herein as a “proposed set of parameters,” “recommended platform adjustments,” “machine learning-derived configuration parameters,” “predicted operational settings,” and/or the like.

In one or more embodiments, the system or service implementing method 200 may function to provide, as input, the one or more model input data objects generated by S220 to one or more machine learning models (e.g., the ensemble of instance assessment machine learning models or the like) trained to assess subscriber-specific configuration parameters and predict optimal platform tuning parameters that adaptively refine workflows, thresholds, compliance rules, or other operational settings of the digital platform (e.g., subscriber-specific instance) for the target subscriber. Stated another way, in one or more embodiments, the one or more machine learning models may assess historical claim data associated with the target subscriber, historical payment data associated with the target subscriber, payer contract data objects associated with the target subscriber, payer contract compliance rules associated with the target subscriber, operational settings (e.g., subscriber-specific platform settings or the like) set by the target subscriber within the digital platform, operational benchmarks set by the target subscriber within the digital platform (e.g., target subscriber-specific performance metrics to be achieved by the digital platform), third-party industry standards, and/or external guidelines to predict a proposed set of platform tuning parameters for the target subscriber. It shall be recognized that the phrase “digital platform of the target subscriber” may be interchangeably referred to herein as a “subscriber-specific instance of the multi-tenant cloud-based service.”

In a first implementation, in response to converting the single data structure into the single model input data object, S230 may function to automatically provide the single model input data object to a single machine learning model (e.g., single instance assessment machine learning model or the like) trained to predict a proposed set of platform tuning parameters for the target subscriber based on assessing the single model input data object, as shown generally by way of example in FIG. 3. In such an implementation, the single model input data object may include a normalized representation of subscriber-specific configuration parameters across all modules and submodules of the digital platform and, in turn, the single machine learning model may collectively assess the subscriber-specific configuration parameters across all modules and submodules of the digital platform as a whole to generate a proposed set of platform tuning parameters that account for cross-module dependencies, system-level behaviors, and overall subscriber performance objectives. Stated another way, in such an implementation, the single machine learning model may simultaneously evaluate the complete set of subscriber-specific configuration parameters included in the single model input data object and generate a proposed set of platform tuning parameters that, when implemented, improves a performance or computing performance of all modules and submodules of the digital platform for the target subscriber.

For instance, in a non-limiting example, one of the proposed set of platform tuning parameters outputted by the single machine learning model may relate to adjusting the current minimum timing-based threshold value specified by the target subscriber within the first module of the digital platform (e.g., from 10 days (e.g., the current minimum timing-based threshold value) to 5 days, from 10 days (e.g., the current minimum timing-based threshold value) to 30 days, etc.).

Additionally, or alternatively, in the non-limiting example, one of the proposed set of platform tuning parameters outputted by the single machine learning model may relate to adjusting the current maximum demand-based threshold value specified by target subscriber within the first module of the digital platform (e.g., from $20 (e.g., the current maximum demand-based threshold value) to $25, from $20 (e.g., the current maximum demand-based threshold value) to $5).

Additionally, or alternatively, in the non-limiting example, one of the proposed set of platform tuning parameters outputted by the single machine learning model may specify a proposed rule change (e.g., proposed computer-executable rule change, etc.). For instance, in a non-limiting example, the proposed rule change may relate to modifying a current coding rule that mandates all laboratory charges to be routed to a coding review queue to instead allow laboratory charges under $50 to bypass the coding review queue and proceed directly to statement processing. In another non-limiting example, the proposed rule change may relate to modifying a current coding rule that exempts radiology charges under $200 from being routed to the coding review queue to instead require radiology charges exceeding $100 to be routed to the coding review queue. In another non-limiting example, the proposed rule change may relate to deleting (e.g., removing, etc.) one or more rule-based heuristics of the plurality of rule-based heuristics used by the second module, adding one or more additional rule-based heuristics to the plurality of rule-based heuristics used by the second module, and/or augmenting one or more rule-based heuristics of the plurality of rule-based heuristics used by the second module.

Additionally, or alternatively, in the non-limiting example, one of the proposed set of platform tuning parameters outputted by the single machine learning model may specify an adaptation to a current automated claim follow-up instruction encoded within an instance of the digital platform that corresponds to the target subscriber. For instance, in a non-limiting example, the adaptation may relate to modifying a current automated instruction that automatically creates and transmits, over a computer network, a follow-up message on all denied claims after 30 days to instead automatically create and transmit, over the computer network, a follow-up message within 10 days for high-dollar claims exceeding a threshold value, while maintaining the 30-day interval for low-dollar claims (e.g., claims not exceeding the threshold value).

Additionally, or alternatively, in the non-limiting example, one of the proposed set of platform tuning parameters outputted by the single machine learning model may specify a proposed parameter change to a payer contract data object. For instance, in a non-limiting example, the proposed parameter change may relate to consolidating multiple payer contract heuristics (e.g., multiple computer-executable payer contract instructions) associated with the same entity (e.g., the same payer) that are stored in separate data objects into a single normalized contract data object. The proposed parameter change may reduce redundant rule evaluations across duplicate or overlapping contract terms, decrease memory utilization during claim adjudication, and improve computational efficiency by minimizing the number of contract objects that must be simultaneously retrieved, loaded, and processed by the digital platform. In another non-limiting example, the proposed parameter change may relate to replacing two or more payer contract heuristics with a single heuristic encoded within the payer contract data object. For instance, rather than storing separate heuristics that (i) require appending a contractual adjustment code when the billed amount exceeds a payer-specific threshold and (ii) require appending a contractual adjustment code when the number of service units exceeds a payer-specific threshold, the system or service implementing method 200 may generate a single heuristic that evaluates both billing amount and service units within one unified logical expression. Such a heuristic consolidation may reduce redundant contract rule executions, decrease memory utilization during claim creation, and improve computational efficiency by streamlining overlapping contractual conditions into a single machine-readable instruction.

Additionally, or alternatively, in the non-limiting example, one of the proposed set of platform tuning parameters outputted by the single machine learning model may specify a modification to one or more components of a computer-executable data acquisition instruction encoded within a third module of the digital platform for the target subscriber. Such a modification may function to resolve anomalies, inconsistencies, or inefficiencies in how subscriber-specific data is retrieved, validated, or integrated from external or internal sources, thereby improving accuracy, consistency, and performance of downstream automated claim creation and automated payment processing operations for the target subscriber.

In a second implementation, in response to generating a plurality of model input data objects that correspond to a plurality of distinct modules of the digital platform, S230 may function to automatically provide the plurality of model input data objects to a plurality of distinct machine learning models (e.g., ensemble of instance assessment machine learning models), wherein each machine learning model is trained to assess subscriber-specific configuration parameters associated with a respective distinct module of the digital platform, as shown generally by way of example in FIG. 4. For instance, in a non-limiting example, a first model input data object controlling a first module of the digital platform may be provided to a first machine learning model (e.g., first instance assessment machine learning model) trained to predict a proposed set of platform tuning parameters for the first module. In such an example, the proposed set of platform tuning parameters may include modifications to timing-based thresholds, escalation rules, or dispositioning parameters that control how the first module transitions pending digital items to closed, terminated, or dispositioned states. By contrast, a second model input data object controlling a second module of the digital platform may be provided to a second machine learning model (e.g., second instance assessment machine learning model) trained to predict a proposed set of platform tuning parameters for the second module, such as adaptations to rule-based heuristics governing coding requirements, exception handling, or workflow routing. Similarly, a third model input data object controlling a third module of the digital platform may be provided to a third machine learning model (e.g., third instance assessment machine learning model) trained to predict a proposed set of platform tuning parameters for the third module, such as modifications to third-party transaction parameters, contract compliance rules, or data acquisition parameters.

It shall be recognized that, in one or more embodiments, S230 may use any suitable machine learning model for predicting a proposed set of platform tuning parameters for a target subscriber including, but not limited to, convolutional neural networks (CNNs), recurrent neural networks (RNNs), transformer-based architectures, natural language processing (NLP) models, large language models (LLMs), gradient boosting machines, random forests, support vector machines (SVMs), logistic regression models, or hybrid ensemble architectures.

In one or more embodiments, based on or in response to providing, as input, the plurality of distinct model input data associated with a subscribing entity to an ensemble of instance assessment machine learning models, S230 may function to generate, using the ensemble of instance assessment machine learning models, a proposed set of parameters for controlling a future runtime behavior of the target subscriber-specific instance corresponding to the subscribing entity.

For instance, with continued reference to the above non-limiting example, in response to S220 generating (i) the first model input data object that includes all threshold-based parameters encoded within the target subscriber-specific instance, (ii) the second model input data object that includes all computer-executable instructions defining automated workflows or machine-controlled operations encoded within the target subscriber-specific instance, (iii) the third model input data object that includes all data acquisition computer procedures encoded within the target subscriber-specific instance, and (iv) the fourth model input data object that includes all rule activation control values encoded within the target subscriber-specific instance, the system or service implementing method 200 may function to provide, as input, the first model input data object, the second model input data object, the third model input data object, and the fourth model input data object to one or more machine learning models included in the ensemble of instance assessment machine learning models.

In such a non-limiting example, the first model input data object may be provided as input to a first instance assessment machine learning model of the ensemble of instance assessment machine learning models and, in turn, the first instance assessment machine learning model may assess the first model input data object and generate a first set of proposed parameters based on the assessment of the first model input data object.

For instance, in such a non-limiting example, the first model input data object may specify at least a current threshold value for a respective minimum time-based threshold. In such a non-limiting example, the target subscriber-specific instance corresponding to the subscribing entity may be configured to automatically route a subject digital item to a disposal queue when a target time-based attribute associated with the subject digital item is equal to or greater than the current threshold value specified for the respective minimum time-based threshold. Accordingly, in response to the first instance assessment machine learning model assessing the first model input data object, the first instance assessment machine learning model may function to output the first set of proposed parameters that specifies a new threshold value for the respective minimum time-based threshold (e.g., the new threshold value may be greater than or less than the current threshold value specified for the respective minimum time-based threshold).

Additionally, or alternatively, in such a non-limiting example, the first model input data object may specify a current threshold value for a respective maximum threshold. In such a non-limiting example, the target subscriber-specific instance corresponding to the subscribing entity may be configured to automatically route a subject digital item to a disposal queue when a target attribute associated with the subject digital item is less than the current threshold value specified for the respective maximum threshold. Accordingly, in response to the first instance assessment machine learning model assessing the first model input data object, the first instance assessment machine learning model may function to output the first set of proposed parameters that specifies a new threshold value for the respective maximum threshold (e.g., the new threshold value is greater than or less than the current threshold value specified for the respective maximum threshold).

Additionally, or alternatively, in such a non-limiting example, the first model input data object may specify a current threshold value for a respective minimum machine learning model prediction threshold. The target subscriber-specific instance, in such a non-limiting example, may be configured to automatically predict, using a first set of machine learning models, a probability of a subject digital item having an adverse decision being reversed, as described in US Patent Application No. 18/901,250, titled “Systems and Method for Automated and Assistive Resolution of Unmapped Patient Intake Data” and an adverse digital claim assessment module as described in US Patent Application No. 18/901,279, titled “Systems and Methods for Machine Learning-Based Routing of Adverse Digital Claims, which are incorporated in their entireties by this reference. A machine learning model prediction threshold, in some embodiments, may refer to a configurable threshold value that defines a minimum predicted probability required to trigger one or more automated actions, such as automatic correction of one or more defects, escalation to a review workflow, or routing of the subject digital item to a particular processing path during runtime of the target subscriber-specific instance. Accordingly, in response to the first instance assessment machine learning model assessing the first model input data object, the first instance assessment machine learning model may function to output the first set of proposed parameters that specifies a new threshold value for the respective minimum machine learning model prediction threshold (e.g., the new threshold value may be greater than or less than the current threshold value specified for the respective minimum machine learning model prediction threshold).

It shall be recognized that, in some embodiments, the first instance assessment machine learning model may be a large language model, a machine learning-based classification model, a machine learning-based regression model, a neural network, a transformer-based machine learning model, a gradient boosting model, a random forest model, or any other suitable machine learning model that may be configured to assess threshold-based subscriber-specific parameters and generate proposed parameter modifications for controlling future runtime behavior of the target subscriber-specific instance.

It shall be further recognized that, in some embodiments, the system or service implementing method 200 may function to source a corpus of training data for configuring the first instance assessment machine learning model. The corpus of training data may include a plurality of training data samples, wherein each distinct training data sample of the plurality of training data samples may include a distinct identifier for a respective threshold, a threshold type corresponding to the respective threshold, a threshold value for the respective threshold, one or more attributes identity a module, workflow, or processing rule to which the respective threshold corresponds, one or more threshold performance labels or performance metrics associated with execution of a respective subscriber-specific instance when the respective threshold was satisfied during runtime of the respective subscriber-specific instance. Stated another way, each training data sample may encode a relationship between a threshold configuration and observed runtime outcomes, thereby enabling the first instance assessment machine learning model to learn how different threshold values and threshold types influence execution behavior and computing performance of subscriber-specific instances. Accordingly, in one or more embodiments, S230 may function to configure the first instance assessment machine learning model based on a training of a target machine learning model (e.g., a large language model, a machine learning-based classification model, a machine learning-based regression model, a neural network, a transformer-based machine learning model, a gradient boosting model (e.g., XGBoost, LightGBM, CatBoost, etc.), a random forest model, or the like) using the corpus of training data. The first instance assessment machine learning model, once trained, may be configured to process threshold-based subscriber-specific parameters associated with a target subscriber-specific instance and generate one or more proposed threshold-related parameter modifications predicted to improve future runtime behavior and computing performance of the target subscriber-specific instance.

Additionally, or alternatively, with continued reference to the above non-limiting example, the second model input data object may be provided as input to a second instance assessment machine learning model of the ensemble of instance assessment machine learning models and, in turn, the second instance assessment machine learning model may assess the second model input data object and generate a second set of proposed parameters based on the assessment of the second model input data object.

For instance, in such a non-limiting example, the second model input data object may specify a first computer-executable medical coding instruction that specifies a first set of medical events that require medical coding and a second computer-executable medical coding instruction that specifies a second set of medical events that do not require medical coding. The target subscriber-specific instance, in such a non-limiting example, may be configured to automatically assess, by executing the first computer-executable medical coding instruction and the second computer-executable medical coding instruction, one or more medical events included in a subject digital item to determine whether the one or more medical events correspond to the first set of medical events or the second set of medical events and, in turn, automatically generate a digital artifact explaining the one or more medical events performed on a respective entity without any medical codes or coding-related information in response to the target subscriber-specific instance detecting that the one or more medical events corresponds to the second set of medical events. Accordingly, in response to the second instance assessment machine learning model assessing the second model input data object, the second instance assessment machine learning model may function to output the second set of proposed parameters that may specify a new iteration of the first computer-executable medical coding instruction and a new iteration of the second computer-executable medical coding instruction. It shall be recognized that, in some embodiments, the new iteration of the first computer-executable medical coding instruction may include one or more additional medical events that require medical coding when compared to the first set of medical events of the first computer-executable medical coding instruction. It shall be further recognized that, in some embodiments, the new iteration of the second computer-executable medical coding instruction may include a reduced set of medical events that do not require medical coding when compared to the second set of medical events of the second computer-executable medical coding instruction.

Additionally, or alternatively, in such a non-limiting example, the second model input data object may specify an automated workflow that controls routing, escalation, or dispositioning of digital items across one or more processing stages of the target subscriber-specific instance. For instance, the automated workflow may specify one or more conditional execution paths that routes a digital item to a first processing queue when a first condition is satisfied and routes the digital item to a second processing queue when a second condition is satisfied. Accordingly, in response to the second instance assessment machine learning model assessing the second model input data object, the second instance assessment machine learning model may function to output the second set of proposed parameters that modifies at least one of the one or more conditional execution paths of the automated workflow. In a non-limiting example, such a modification may include consolidating two or more sequential workflow steps into a single execution step, reordering execution of workflow steps to reduce redundant processing, or removing a workflow step that is determined to be low-value or unnecessary based on observed runtime outcomes.

Additionally, or alternatively, in such a non-limiting example, the second set of proposed parameters generated by the second instance assessment machine learning model may specify activating, deactivating, or conditionally executing one or more computer-executable instructions associated with automated decision-making, exception handling, or escalation logic. Such proposed modifications may reduce repeated evaluation of overlapping instructions, decrease execution branching, and improve processing efficiency of the target subscriber-specific instance during runtime.

Additionally, or alternatively, the second instance assessment machine learning model may generate proposed parameters that modify associations between automated workflows and threshold-based routing logic, such that automated workflow execution is conditionally triggered only when a subset of thresholds or threshold conditions is satisfied, thereby reducing unnecessary automated workflow invocation and improving overall execution efficiency.

It shall be recognized that, in some embodiments, the second instance assessment machine learning model may be a large language model, a machine learning-based classification model, a machine learning-based regression model, a neural network, a transformer-based machine learning model, a gradient boosting model, a random forest model, or any other suitable machine learning model that may be configured to assess computer-executable instructions and generate proposed modifications to the computer-executable instructions.

It shall be further recognized that, in some embodiments, the system or service implementing method 200 may function to source a corpus of training data for configuring the second instance assessment machine learning model. The corpus of training data may include a plurality of training data samples associated with automated workflows, routing logic, escalation logic, and other computer-executable instructions executed by subscriber-specific instances of the multi-tenant cloud-based service. In one or more embodiments, each training data sample of the corpus of training data may include a respective identifier corresponding to a respective automated workflow or computer-executable instruction, one or more representations of execution logic associated with the respective automated workflow (e.g., conditional paths, step ordering, or instruction dependencies), one or more attributes identifying a module or processing context in which the respective automated workflow is executed, and one or more observed performance metrics associated with execution of the respective automated workflow during runtime of a subject subscriber-specific instance.

Stated another way, each training data sample may encode a relationship between an automated workflow configuration and observed runtime behavior of the subscriber-specific instance, such as processing latency, routing frequency, escalation rates, exception rates, or resource utilization characteristics. Accordingly, in one or more embodiments, S230 may function to configure the second instance assessment machine learning model based on a training of a target machine learning model (e.g., a large language model, a machine learning-based classification model, a machine learning-based regression model, a neural network, a transformer-based machine learning model, a gradient boosting model (e.g., XGBoost/LightGBM/CatBoost), a random forest model, or the like) using the corpus of training data. The second instance assessment machine learning model, once trained, may be configured to process computer-executable instructions associated with a target subscriber-specific instance and generate one or more proposed modifications to the computer-executable instructions predicted to improve future runtime behavior and computing performance of the target subscriber-specific instance.

Additionally, or alternatively, with continued reference to the above non-limiting example, the third model input data object may be provided as input to a third instance assessment machine learning model of the ensemble of instance assessment machine learning models and, in turn, the third instance assessment machine learning model may assess the third model input data object and generate a third set of proposed parameters based on the assessment of the third model input data object.

For instance, in such a non-limiting example, the third model input data object may specify a data acquisition computer procedure that the target subscriber-specific instance invokes to periodically fetch data from one or more electronic health record systems. The third instance assessment machine learning model may detect an anomaly or misconfiguration in the data acquisition computer procedure, and, in turn, the third instance assessment machine learning model may generate the third set of proposed parameters that specifies a new data acquisition computer procedure that corrects the anomaly or misconfiguration detected in the data acquisition computer procedure. It shall be noted that the anomaly or misconfiguration detected in the data acquisition computer procedure may include or relate to at least a missing or incomplete parameter required for periodically fetching the data from the one or more electronic health record systems. It shall be further recognized that the new data acquisition computer procedure may include a parameter value for the missing or incomplete parameter.

It shall be recognized that, in some embodiments, the third instance assessment machine learning model may be a large language model, a machine learning-based classification model, a machine learning-based regression model, a neural network, a transformer-based machine learning model, a gradient boosting model, a random forest model, or any other suitable machine learning model that may be configured to assess data acquisition computer procedures associated with a target subscriber-specific instance and generate one or more proposed modifications to the data acquisition computer procedures predicted to improve accuracy, reliability, and computing performance of data retrieval operations during runtime of the target subscriber-specific instance.

It shall be further recognized that, in some embodiments, the system or service implementing method 200 may function to source a corpus of training data for configuring the third instance assessment machine learning model. The corpus of training data may include a plurality of training data samples associated with data acquisition computer procedures executed by subscriber-specific instances to retrieve data from one or more electronic health record systems or other external data sources. In one or more embodiments, each training data sample of the corpus of training data may include one or more identifiers corresponding to a respective data acquisition computer procedure, one or more procedure parameters (e.g., scheduling parameters, endpoint identifiers, authentication parameters, or data formatting parameters) of the respective data acquisition computer procedure, one or more attributes identifying a data source or retrieval context associated with the respective data acquisition computer procedure, and one or more observed performance metrics associated with execution of the respective data acquisition computer procedure during runtime of a subscriber-specific instance, such as data retrieval latency, error rates, data completeness metrics, or retry frequency. Accordingly, in one or more embodiments, S230 may function to configure the third instance assessment machine learning model based on training a target machine learning model (e.g., a large language model, a machine learning-based classification model, a machine learning-based regression model, a neural network, a transformer-based machine learning model, a gradient boosting model (e.g., XGBoost/LightGBM/CatBoost), a random forest model, or the like) using the corpus of training data, such that the third instance assessment machine learning model, once trained, is configured to assess data acquisition computer procedures associated with a target subscriber-specific instance and generate one or more proposed modifications to the data acquisition computer procedures predicted to improve reliability, efficiency, and computing performance of data retrieval operations during runtime.

Additionally, or alternatively, with continued reference to the above non-limiting example, the fourth model input data object may be provided as input to a fourth instance assessment machine learning model of the ensemble of instance assessment machine learning models and, in turn, the fourth instance assessment machine learning model may assess the fourth model input data object and generate a fourth set of proposed parameters based on the assessment of the third model input data object.

For instance, in such a non-limiting example, the fourth model input data object may specify one or more rule activation control values associated with one or more computer-executable rules, heuristics, or policies encoded within the target subscriber-specific instance. A respective rule activation control value may indicate whether a corresponding computer-executable rule is enabled, disabled, conditionally enabled, or otherwise selectively applied during runtime of the target subscriber-specific instance. Accordingly, in response to the fourth instance assessment machine learning model assessing the fourth model input data object, the fourth instance assessment machine learning model may function to output the fourth set of proposed parameters that specifies modifying one or more rule activation control values. In a non-limiting example, the fourth set of proposed parameters may specify activating a first computer-executable rule disabled within the target subscriber-specific instance, deactivating a second computer-executable rule enabled within the target subscriber-specific instance, or conditionally enabling execution of a third computer-executable rule disabled within the target subscriber-specific instance.

Additionally, or alternatively, in such a non-limiting example, the fourth model input data object may specify a rule activation control value for a respective computer-executable rule (e.g., the rule activation control value is set to a first value in the one or more corpora of subscriber-specific parameters). The respective computer-executable rule, in such an embodiment, may be deactivated in the target subscriber-specific instance based on the rule activation control value for the respective computer-executable rule being set to the first value in the one or more corpora of subscriber-specific parameters. The fourth proposed set of parameters generated by the fourth instance assessment machine learning model may specify or recommend that the rule activation control value for the respective computer-executable rule be set to a second value different from the first value.

It shall be recognized that, in some embodiments, the fourth instance assessment machine learning model may be a large language model, a machine learning-based classification model, a machine learning-based regression model, a neural network, a transformer-based machine learning model, a gradient boosting model, a random forest model, or any other suitable machine learning model that may be configured to assess rule activation control values governing execution of computer-executable rules encoded within a target subscriber-specific instance and generate one or more proposed modifications to the rule activation control values that activate, deactivate, or conditionally enable execution of the computer-executable rules during runtime.

It shall be further recognized that, in some embodiments, the system or service implementing method 200 may function to source a corpus of training data for configuring the fourth instance assessment machine learning model. The corpus of training data may include a plurality of training data samples associated with execution of computer-executable rules governed by rule activation control values within subscriber-specific instances of the multi-tenant cloud-based service. In one or more embodiments, each training data sample of the corpus of training data may include one or more identifiers corresponding to a respective computer-executable rule, a rule activation control value associated with the respective computer-executable rule, one or more attributes identifying a module, workflow, or processing context in which the computer-executable rule is stored, evaluated, and/or executed, and one or more observed performance metrics associated with execution or non-execution of the computer-executable rule during runtime of a subscriber-specific instance. Such performance metrics may include, without limitation, rule evaluation frequency, execution latency, branching frequency, exception rates, or processor and memory utilization associated with executing the computer-executable rule. Accordingly, in one or more embodiments, S230 may function to configure the fourth instance assessment machine learning model based on training a target machine learning model (e.g., a large language model, a machine learning-based classification model, a machine learning-based regression model, a neural network, a transformer-based machine learning model, a gradient boosting model (e.g., XGBoost/LightGBM/CatBoost), a random forest model, or the like) using the corpus of training data, such that the fourth instance assessment machine learning model, once trained, is configured to process rule activation control values associated with a target subscriber-specific instance and generate one or more proposed modifications to the rule activation control values predicted to improve future runtime behavior and computing performance of the target subscriber-specific instance.

It shall be recognized that the ensemble of instance assessment machine learning models may include and/or use any of the machine learning models described herein without departing from the scope of the disclosure.

It shall be further recognized that, in one or more embodiments, the ensemble of instance assessment machine learning models may generate the proposed set of parameters based on assessing the plurality of model input data objects against one or more of historical claim and payment data associated with the target subscriber-specific instance of the multi-tenant cloud-based service, a plurality of distinct payer contract data objects that each specify one or more payer-specific rules, one or more payer-specific reimbursement policies, and one or more payer-specific compliance requirements applicable to digital items processed by the target subscriber-specific instance, machine-readable industry standard specification data obtained from one or more external data sources, one or more machine-readable industry standard rule sets obtained from the one or more external data sources, and historical benchmark data of the subscribing entity that corresponds to the target subscriber-specific instance.

In one or more embodiments, the system or service implementing method 200 may be configured to operate in at least two distinct assessment and remediation scenarios. In a first scenario, the system or service may perform, using the ensemble of instance assessment machine learning models, assessment of a pending digital item in response to an explicit user-initiated input received via a graphical user interface, a chatbot interface, or the like, such as a request to analyze, evaluate, or tune a target subscriber-specific instance or a pending digital item associated therewith. In a second scenario, the system or service may perform, using the ensemble of instance assessment machine learning models, assessment autonomously via a background monitoring agent without requiring an explicit user-initiated request. In such embodiments, the background monitoring agent may periodically or continuously monitor operational data, rule activations, parameter values, and detected outcomes associated with the target subscriber-specific instance to identify defects, errors, and/or anomalies and to generate a proposed set of parameters for a pending digital item. Depending on satisfaction of one or more criteria, including confidence thresholds, safety constraints, or auditability constraints, the system or service may either generate a notification requesting user authorization to implement the proposed set of parameters or automatically implement the proposed set of parameters for the pending digital item without user intervention.

Stated differently, in one or more embodiments, the system or service may be configured to selectively initiate assessment and remediation of a pending digital item either responsively based on an explicit user interaction or proactively via an autonomous background monitoring agent, such that detection, recommendation generation, and implementation of corrective actions may occur with or without direct user initiation depending on operational context and satisfaction of predefined criteria.

### Executing one or more Computer-Based Simulations using the Proposed Set of Platform Tuning Parameters

S240, which includes executing computer-based simulations, may function to execute one or more computer-based simulations based on the proposed set of platform tuning parameters outputted by S230. A computer-based simulation, as generally referred to herein, may refer to a computational execution of subscriber-specific workflows, rules, or operations of the digital platform or a respective subscriber-specific instance in a controlled virtual environment using the proposed set of platform tuning parameters as inputs.

In one or more embodiments, S240 may function to execute one or more computer-based simulations to simulate behavioral changes of the digital platform when the digital platform is operating according to the proposed set of platform tuning parameters relative to when the digital platform is operating according to the at least one corpus of subscriber-specific configuration parameters. Stated another way, in one or more embodiments, S240 may function to execute one or more computer-based simulations to simulate behavioral changes of the subscriber-specific instance when the subscriber-specific instance is operating according to the proposed set of parameters relative to when the subscriber-specific instance is operating according to the one or more corpora of subscriber-specific parameters or at least a subset of the one or more corpora of subscriber-specific parameters.

In one or more embodiments, in response to S240 generating a single proposed set of platform tuning parameters that includes at least one system-recommended parameter change and/or at least one system-recommended rule change for each module of the digital platform, S240 may function to execute a single computer-based simulation that assesses a performance of all modules of the digital platform when the digital platform is configured according to the proposed set of platform tuning parameters. For instance, in a non-limiting example, the single computer-based simulation, when executed, may function to execute the computer-executable workflows, computer-executable rules, and computer-executable processes of the digital platform according to the proposed set of platform tuning parameters for a target period (e.g., past year, past quarter, past month) and compute corresponding performance metrics that indicate how the digital platform operated under the proposed or system-recommended configuration (e.g., the single proposed set of platform tuning parameters).

It shall be recognized that, in such an embodiment, the corresponding performance metrics that reflect how the digital platform operated under the proposed configuration may include one or more of a total number of operations executed, average processing times, success or completion rates, error or exception frequencies, resource utilization levels, throughput measurements across one or more modules of the digital platform, a collection efficacy metric, and a denial rate efficacy metric. A collection efficacy metric, as generally referred to herein, may represent a measure of the proportion of total receivables successfully collected within a defined time span relative to the total receivables generated. A denial rate efficacy metric, as generally referred to herein, may represent a measure of the proportion of total submitted claims or items that are rejected, denied, or returned relative to the total number of transactions or claims submitted.

Alternatively, in one or more embodiments, S240 may function to execute a distinct computer-based simulation for each module of the digital platform. In such an embodiment, each module may be individually simulated under the proposed set of platform tuning parameters applicable to that module, while maintaining the existing subscriber-specific configuration parameters for the remaining modules. Each distinct simulation may therefore function to isolate the impact of the proposed parameter changes for a single module, compute performance metrics specific to that module (e.g., processing throughput, execution accuracy, latency reduction, compliance adherence, denial rate efficacy metric, collection efficacy metric, etc.), and generate module-level efficacy indicators indicative of how the individual module would operate under the proposed configuration.

For instance, in a non-limiting example, S240 may function to execute, in parallel, a first distinct computer-based simulation for the first module when the first module is encoded according to a first subset of the proposed set of platform tuning parameters corresponding to the first module, a second distinct computer-based simulation for the second module when the second module is encoded according to a second subset of the proposed set of platform tuning parameters corresponding to the second module, a third distinct computer-based simulation for the third module when the third module is encoded according to a third subset of the proposed set of platform tuning parameters corresponding to the third module, and so forth. Accordingly, in response to simultaneously executing the first distinct computer-based simulation, the second distinct computer-based simulation, the third distinct computer-based simulation, and so forth, S240 may function to compute a distinct set of performance metrics for each distinct module of the digital platform that indicates how the digital platform operated under the proposed configuration for the corresponding module.

Accordingly, in one or more embodiments, S240 may function to output a set of performance metrics (e.g., simulation results, simulations findings, or the like) for each distinct module of the digital platform, as well as aggregated performance metrics for the digital platform as a whole, together with corresponding rationale and evidentiary data that substantiate the impact of the proposed platform tuning parameters.

It shall be recognized that, in some embodiments, the simulation process may operate by applying, executing, and/or implementing the proposed set of platform tuning parameters against a target dataset of historical operational events associated with the digital platform and then executing, in a controlled virtual environment, the computer-executable workflows, rules, and processes of the digital platform in accordance with the proposed configuration. During execution, the simulation process may generate synthetic operational outcomes that mirror how the digital platform would have behaved if the proposed platform tuning parameters had been active during the period corresponding to the target dataset. The synthetic operational outcomes may be logged, analyzed, and transformed into quantitative performance metrics that reflect changes in efficiency, accuracy, compliance, throughput, collection efficacy, denial rate efficacy, or any other suitable indicator of platform performance.

In one or more embodiments, in response to the system or service implementing method 200 generating the proposed set of parameters, S240 may function to execute one or more computer-based simulations simulating an operation of the target subscriber-specific instance configured according to the proposed set of parameters. In one or more embodiments, in response to executing the one or more computer-based simulations, the system or service implementing method 200 may function to display, on a graphical user interface, the proposed set of parameters in association with simulation findings outputted by the one or more computer-based simulations.

In one or more embodiments, executing the one or more computer-based simulations simulating the operation of the target subscriber-specific instance configured according to the proposed set of parameters may include providing a test set of digital items to the target subscriber-specific instance configured according to the proposed set of parameters and computing a first corpus of simulation findings based on processing the test set of digital items using the target subscriber-specific instance configured according to the proposed set of parameters. It shall be recognized that, in such an embodiment, the simulation findings outputted by the one or more computer-based simulations may include the second corpus of simulation findings.

Additionally, in such an embodiment, the system or service implementing method 200 may function to execute an additional computer-based simulation simulating operation of the target subscriber-specific instance configured according to a subset of the one or more corpora of subscriber-specific parameters controlling a runtime of the target subscriber-specific instance. In one or more embodiments, executing the additional computer-based simulation may include providing the test set of digital items to the target subscriber-specific instance configured according to the subset of the one or more corpora of subscriber-specific parameters and computing a second corpus of simulation findings based on processing the test set of digital items using the target subscriber-specific instance configured according to the subset of the one or more corpora of subscriber-specific parameters.

It shall be recognized that, in one or more embodiments, the system or service implementing method 200 may function to receive, while displaying the graphical user interface, an input from a user selecting a tune instance button displayed on the graphical user interface. Accordingly, in response to the system or service implementing method 200 receiving the input from the user selecting the tune instance button, the system or service implementing method 200 may function to automatically execute an instance adaption process that adapts, in real-time or near real-time, the target subscriber-specific instance to an adapted subscriber-specific instance. An instance adaption process may refer to an automated process that modifies subscriber-specific parameters, computer-executable instructions, rule activation control values, or other runtime configuration settings of the target subscriber-specific instance in accordance with the proposed set of parameters or a user-modified version of the proposed set of parameters to produce the adapted subscriber-specific instance. It shall be recognized that all future automated digital actions associated with the user that occur within a target time span (e.g., next seven days, etc.) may be executed using the adapted subscriber-specific instance instead of the target subscriber-specific instance.

In one or more embodiments, the system or service implementing method 200 may function to detect, in real-time using one or more processors, that a difference between an efficacy metric value computed for a subject efficacy metric during the target time span and a benchmark efficacy value defined for the subject efficacy metric exceeds a predefined deviation threshold. Accordingly, in response to detecting that the difference exceeds the predefined deviation threshold, the system or service implementing method 200 may function to generate, in real-time, an alert indicating that the adapted subscriber-specific instance is behaving anomalously with respect to the subject efficacy metric. Furthermore, in such an embodiment, the system or service implementing method 200 may function to transmit, in real-time over a computer network, the alert to a subscribing entity to which the adapted subscriber-specific instance corresponds.

### Surfacing Proposed Set of Platform Tuning Parameters in association with Simulation Findings using a Graphical User Interface

S250, which includes instantiating a graphical user interface, may function to instantiate a graphical user interface that surfaces the proposed set of platform tuning parameters generated for the target subscriber in association with the corresponding simulation findings computed for the target subscriber.

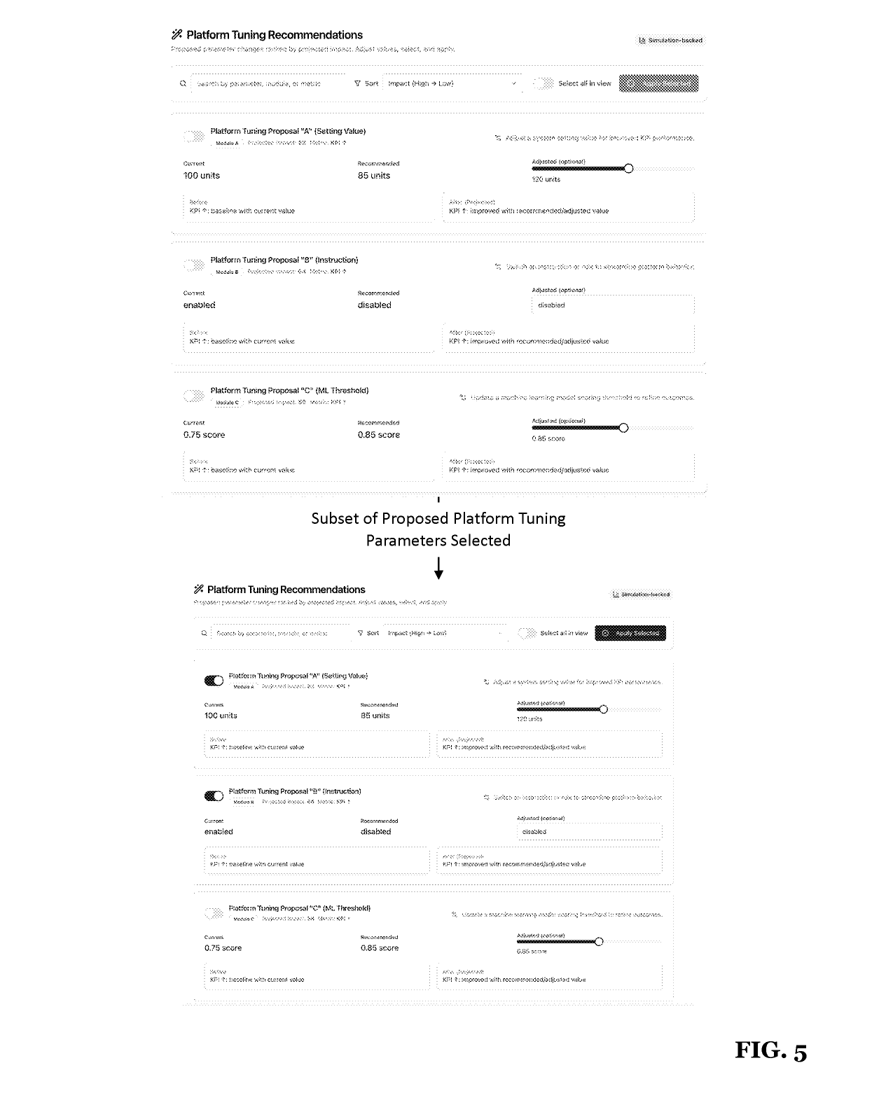

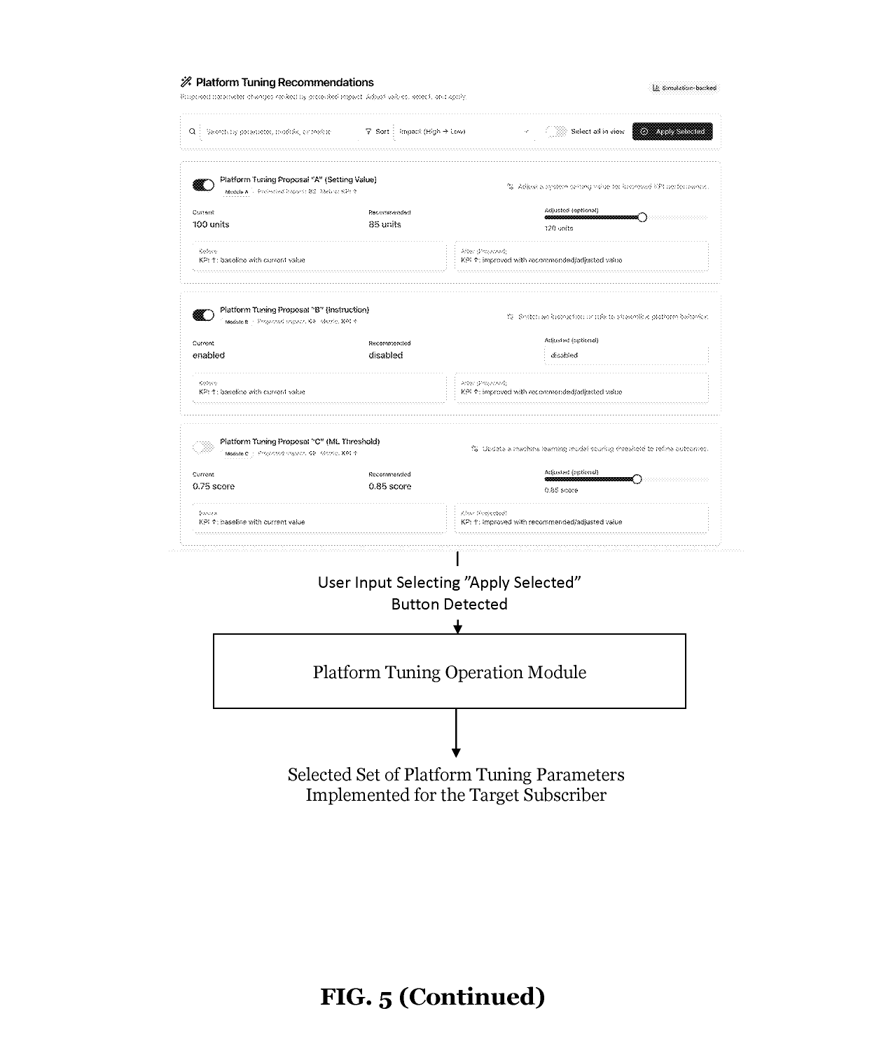

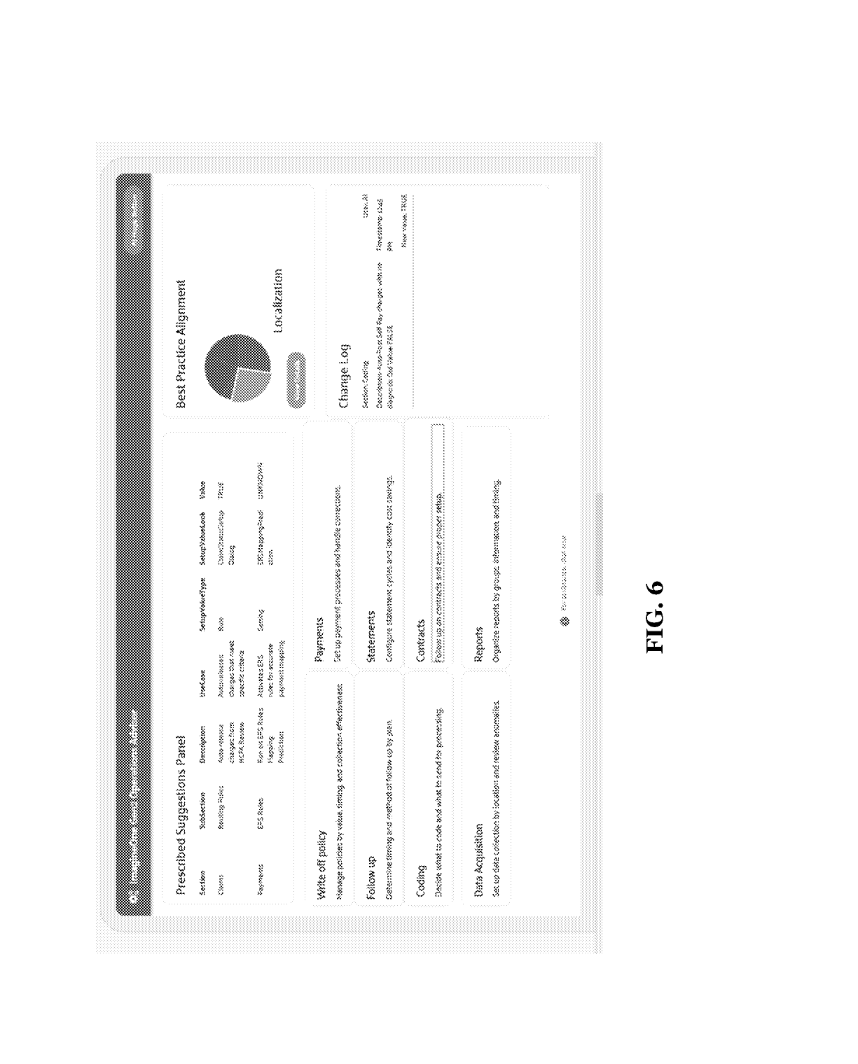

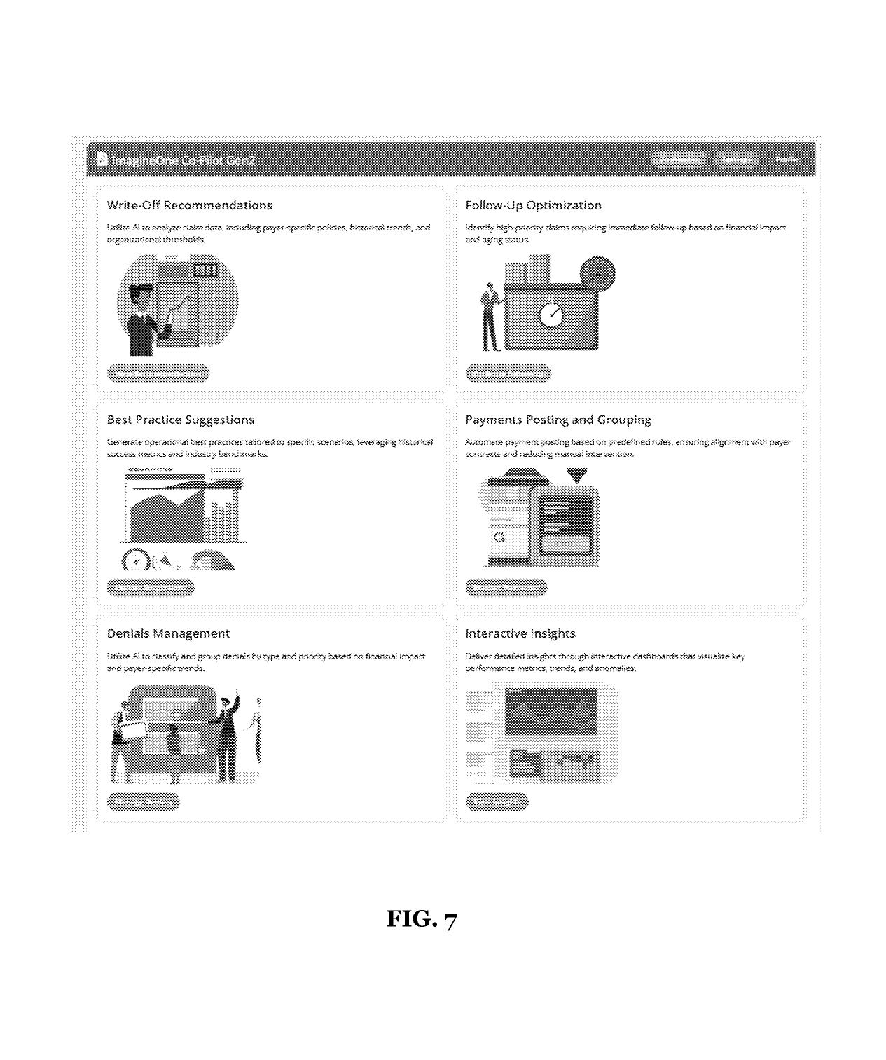

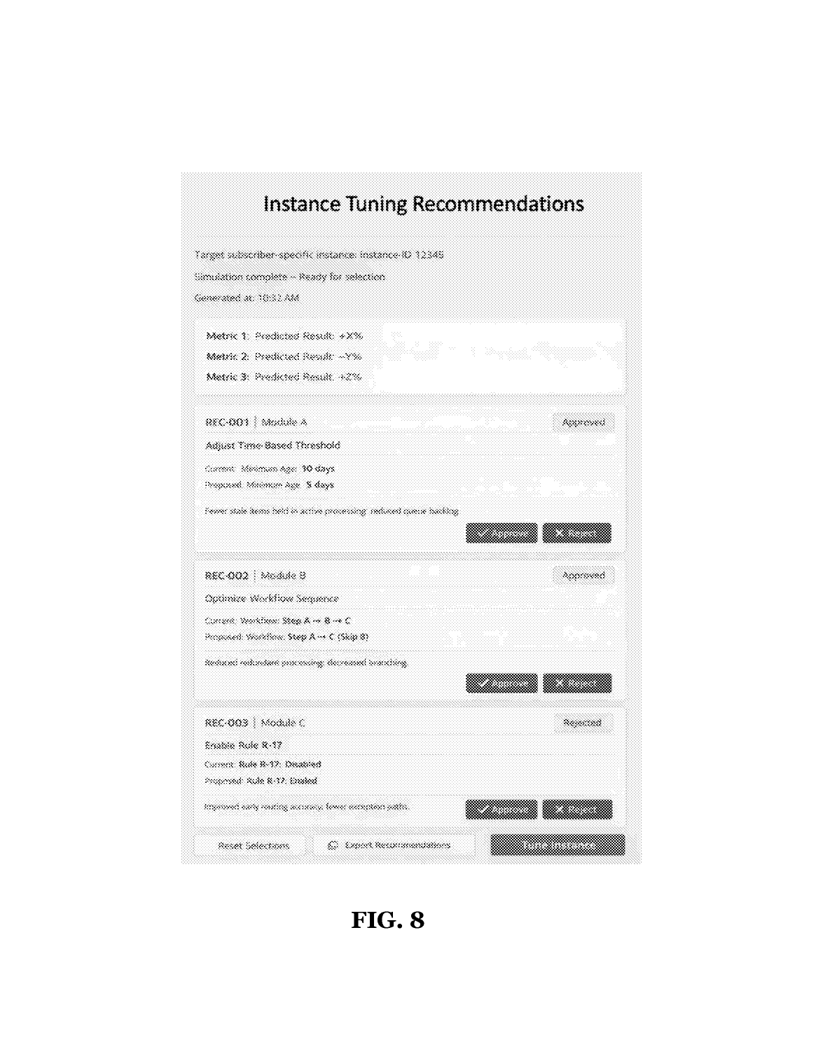

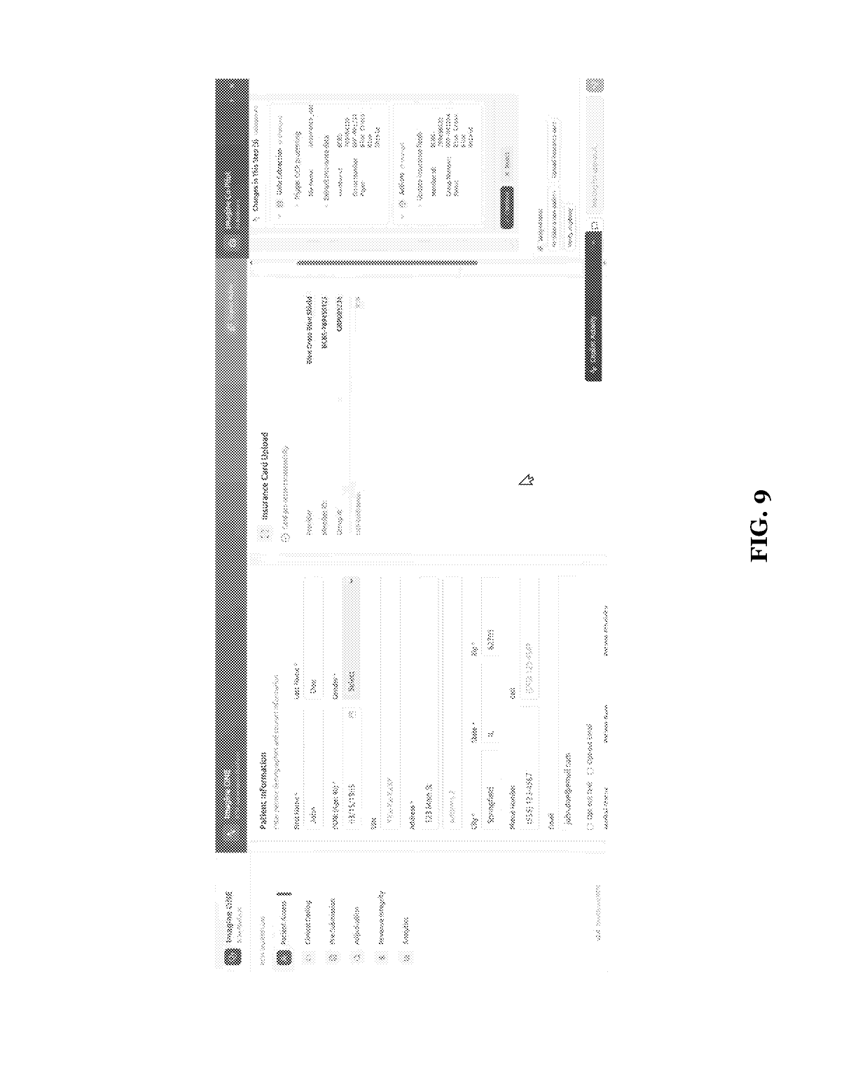

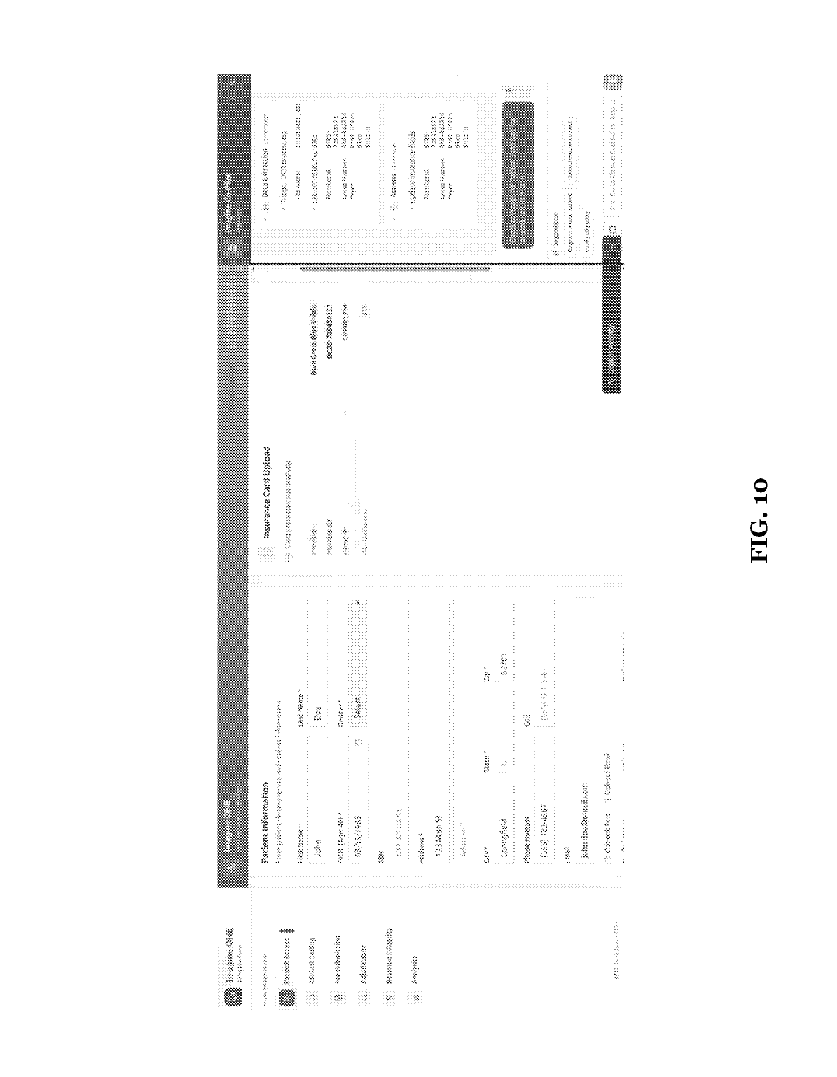

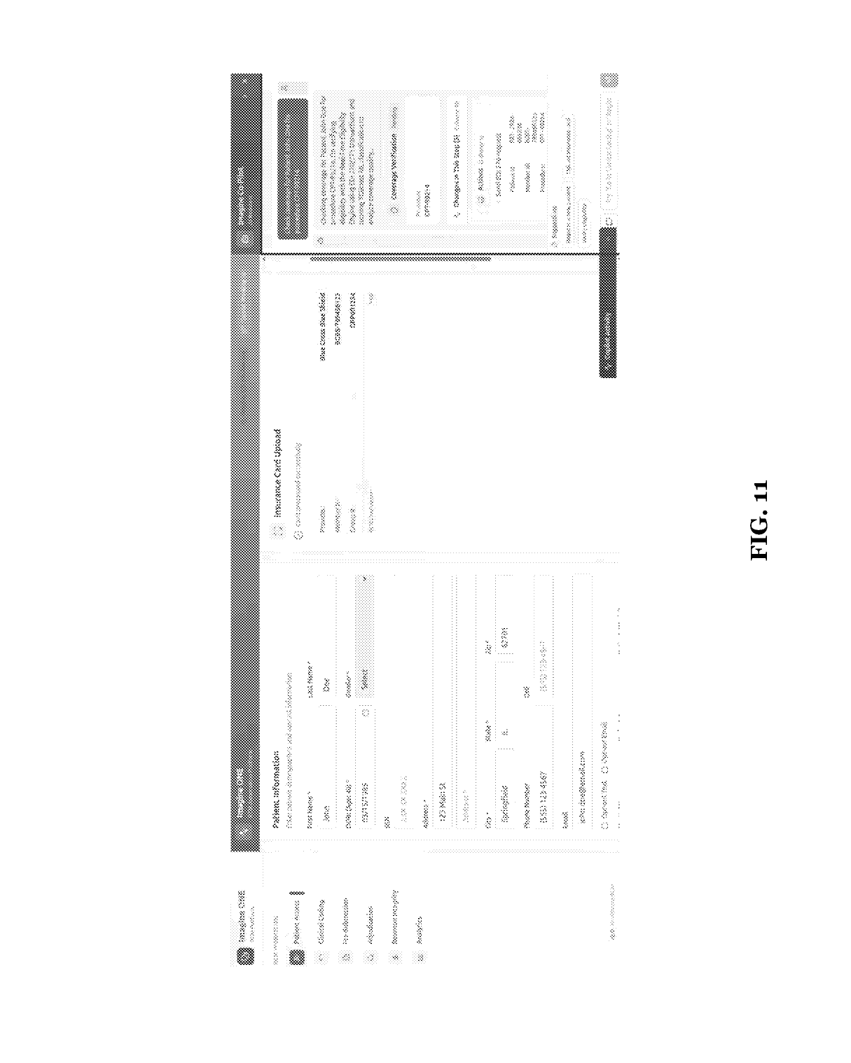

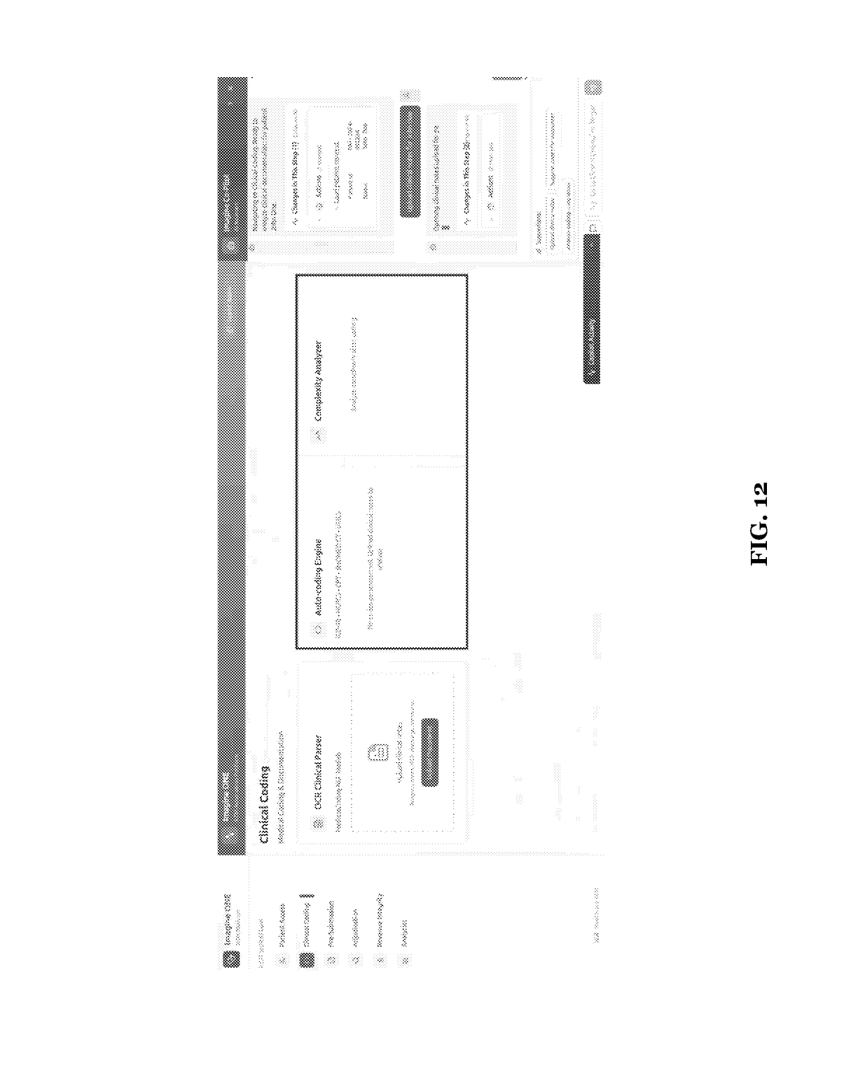

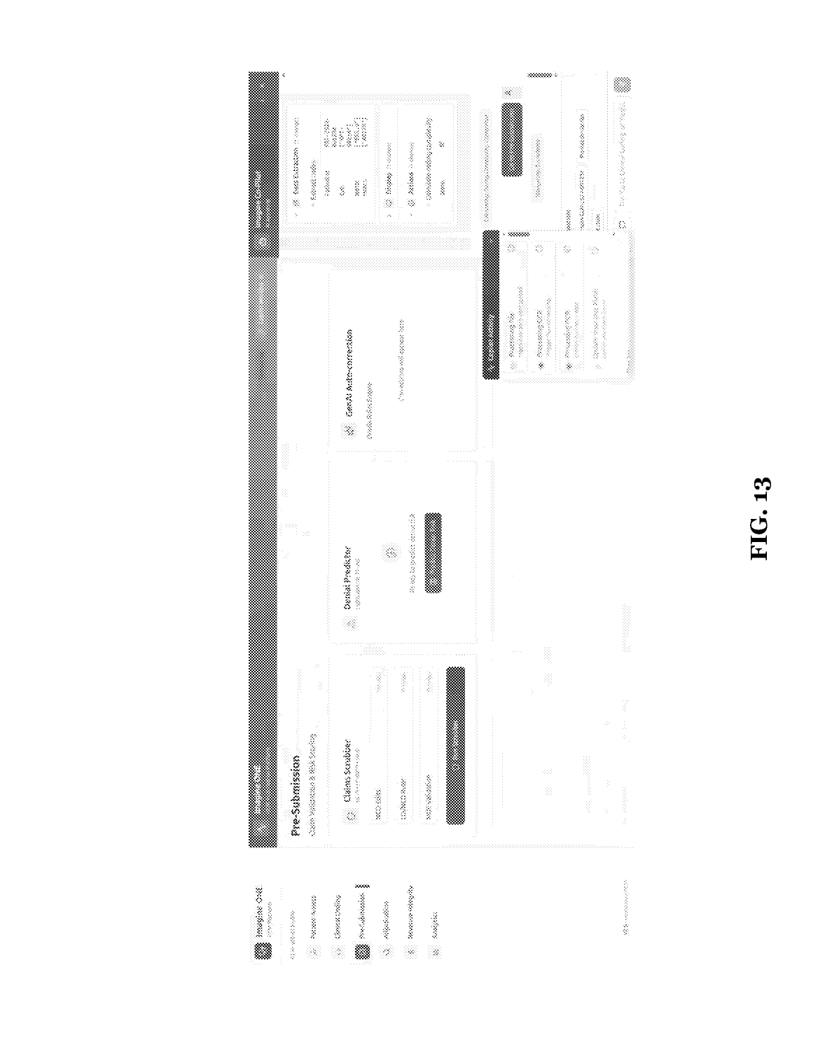

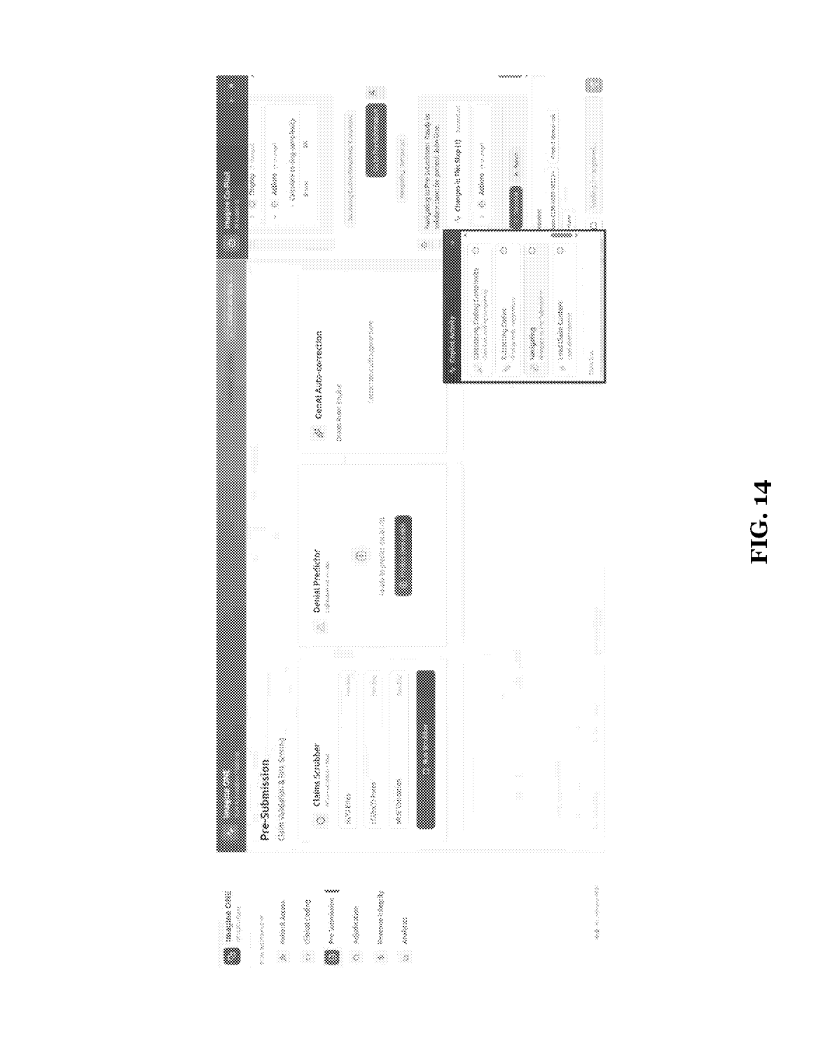

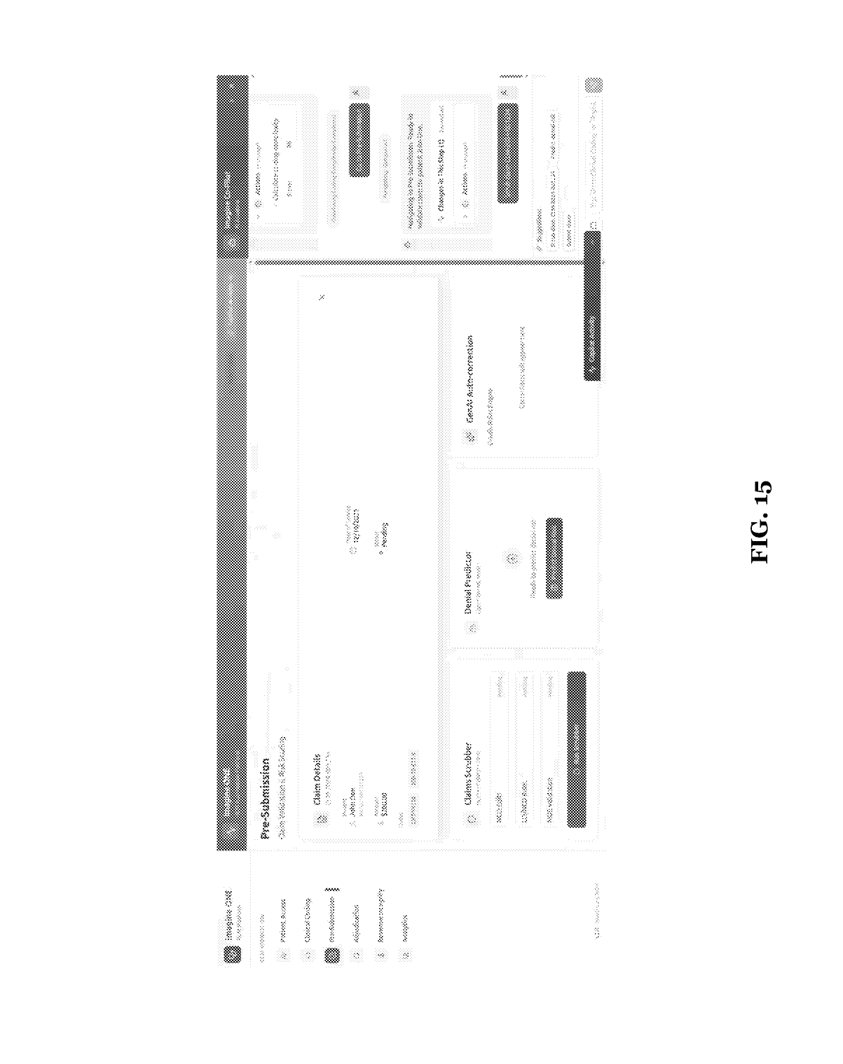

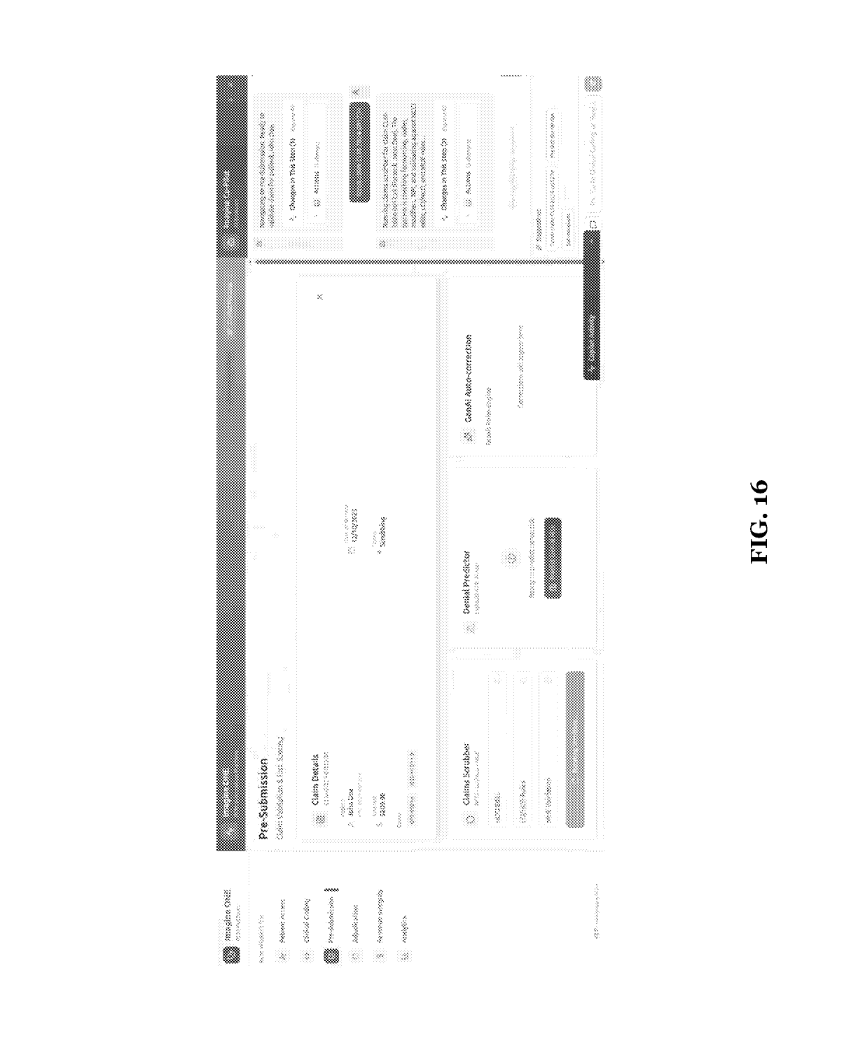

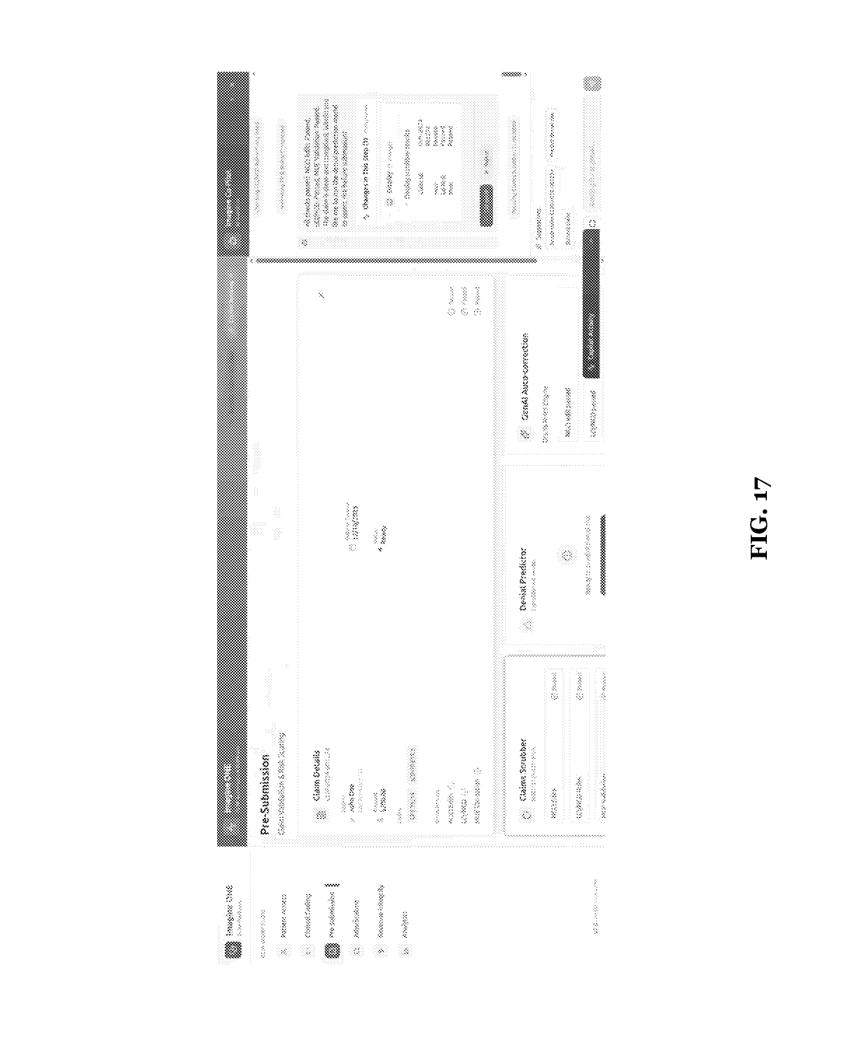

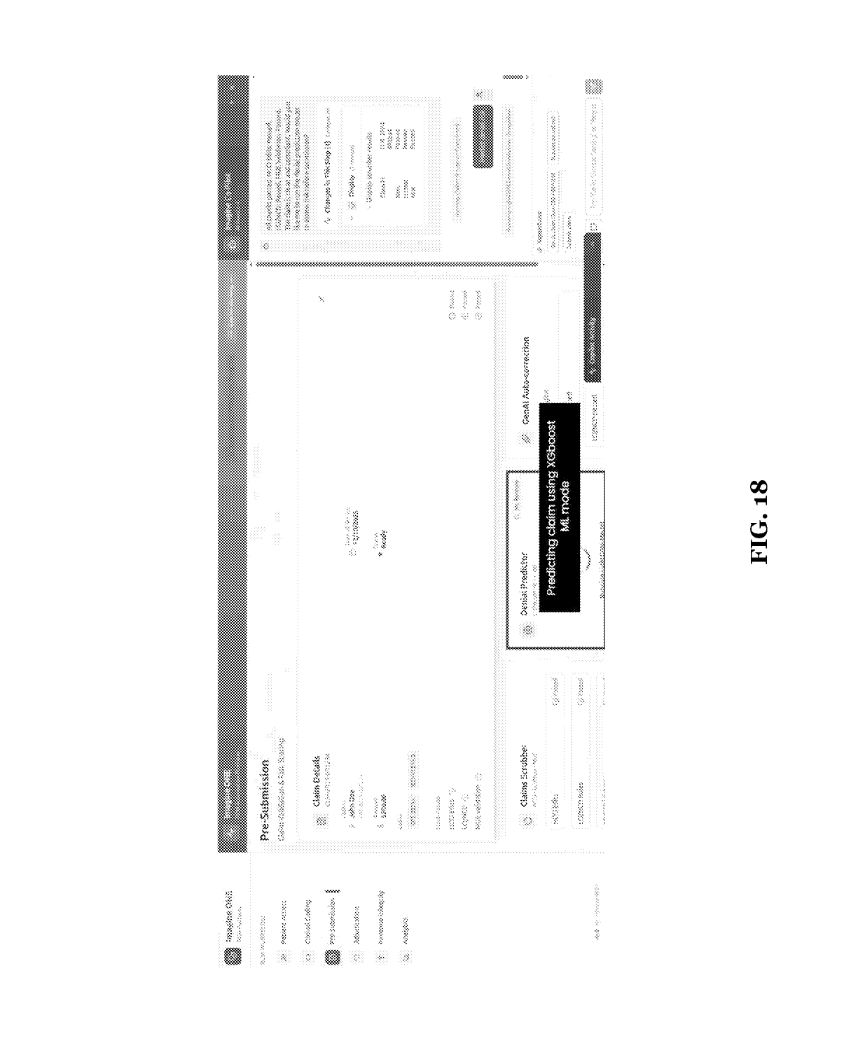

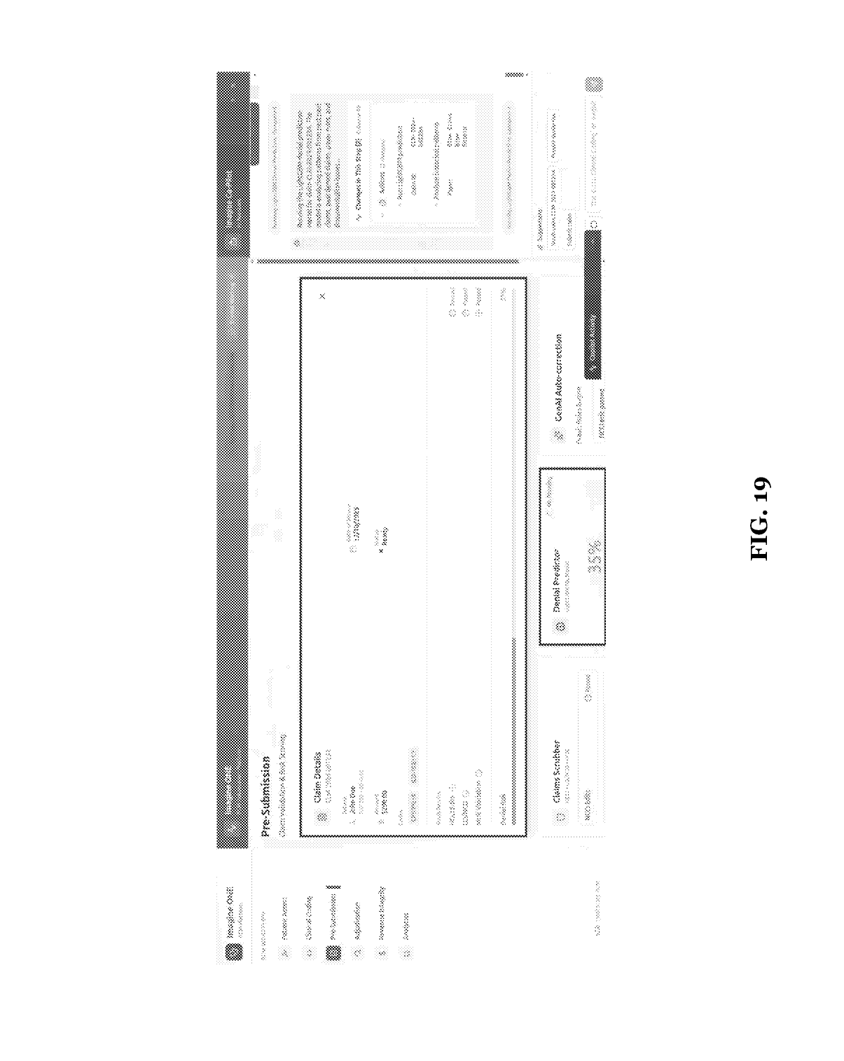

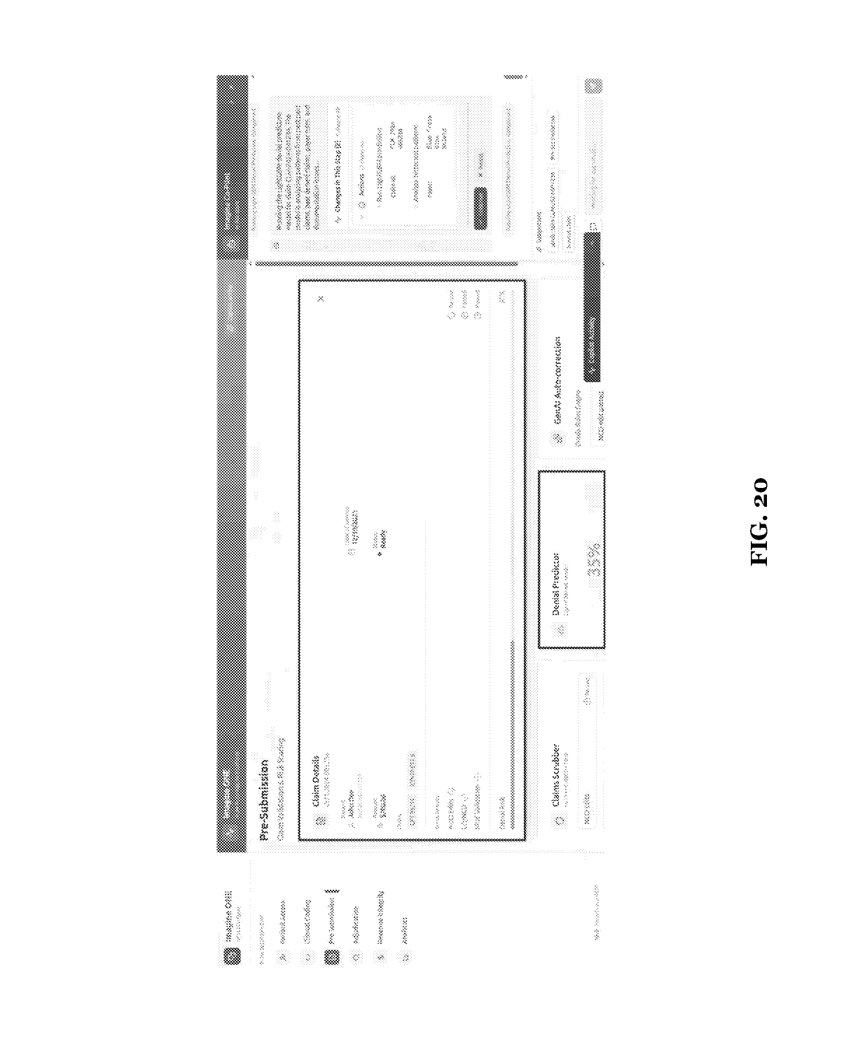

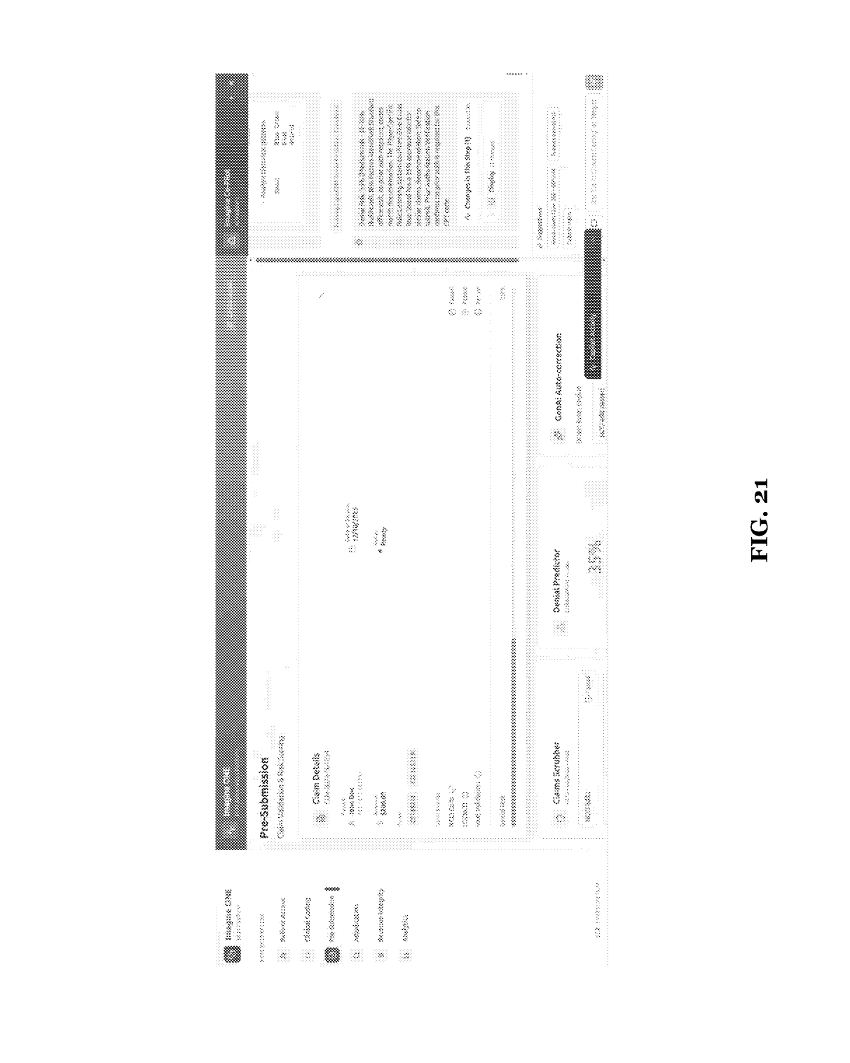

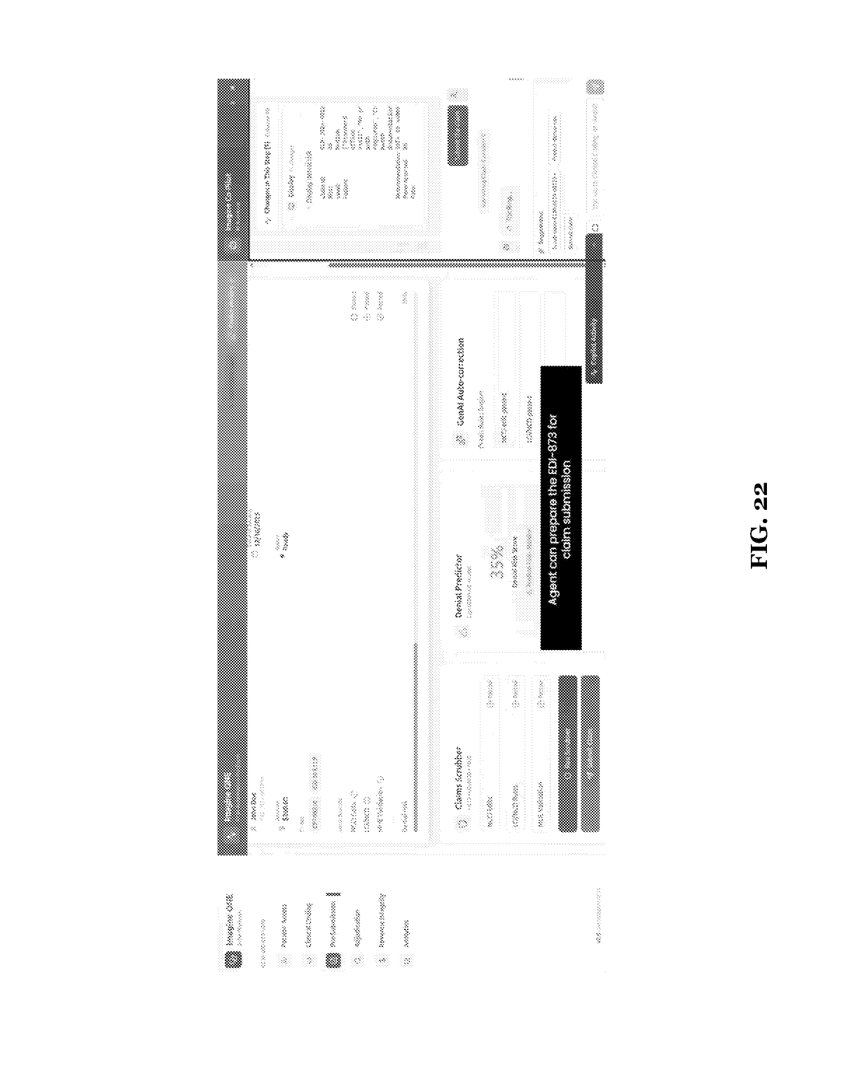

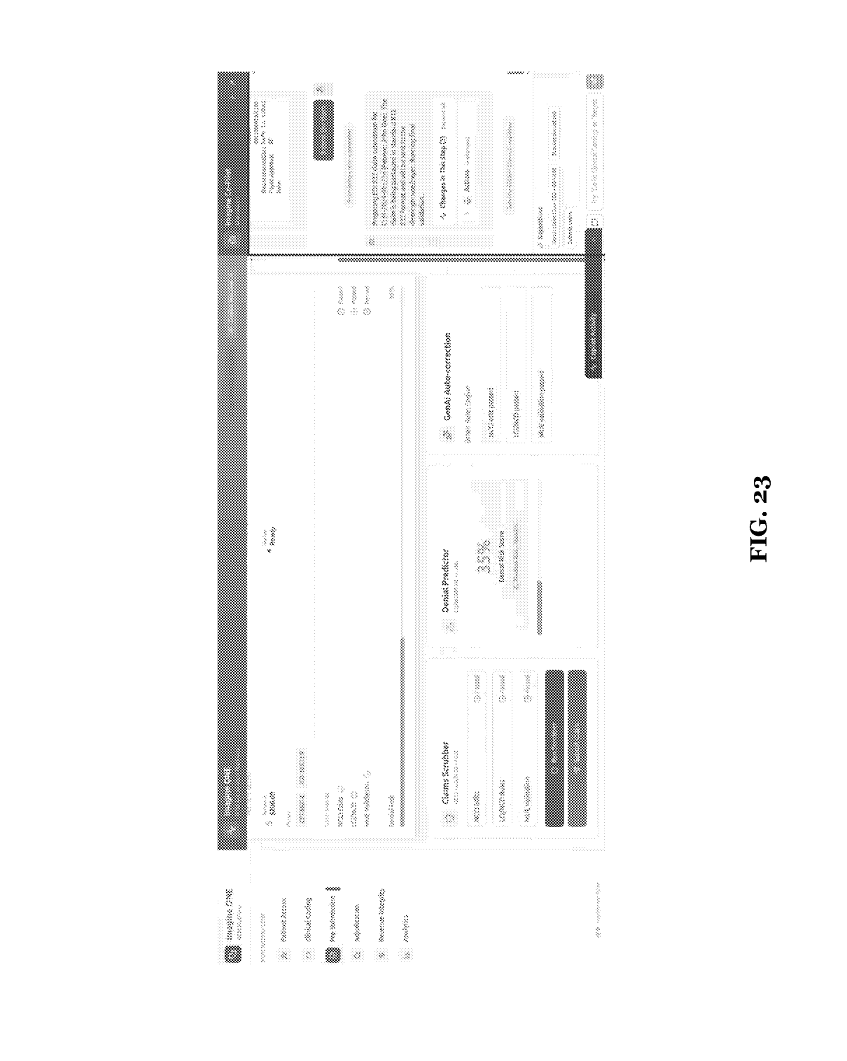

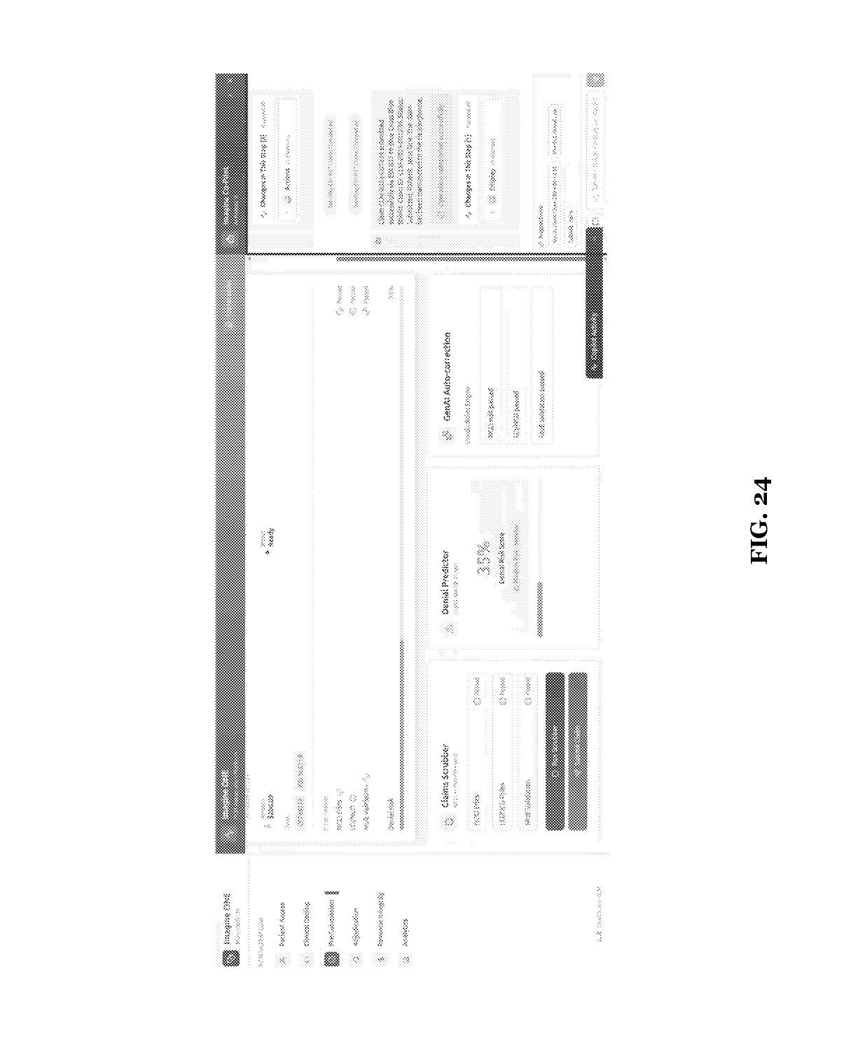

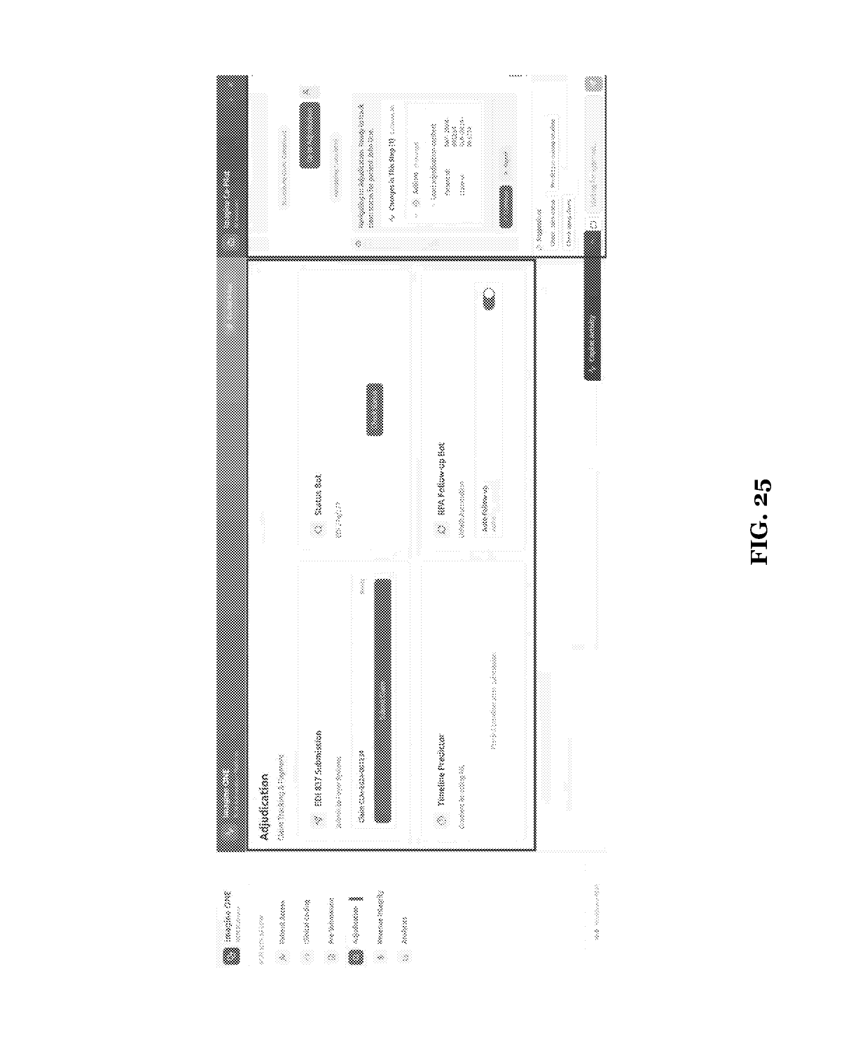

In one or more embodiments, in response to generating the proposed set of platform tuning parameters for the target subscriber and executing the one or more computer-based simulations based on the proposed set of platform tuning parameters generated for the target subscriber, S250 may function to instantiate, in real-time or near real-time, a graphical user interface that displays the proposed set of platform tuning parameters generated for the target subscriber in association with the simulation results or simulation findings outputted by the one or more computer-based simulations, as shown generally by way of example in FIG. 5, FIG. 6, and FIG. 7.

In one or more embodiments, the graphical user interface may function to display a list of platform tuning recommendations displayed within the graphical user interface. In such an embodiment, platform tuning recommendations included in the list may be arranged according to one or more prioritization criteria, such as predicted platform performance improvement, projected decrease in denial rate, projected increase in collection rate, and/or any other suitable platform performance efficacy metric. Stated another way, in some embodiments, the prioritization may be based on simulation findings (e.g., simulation results) that quantify the magnitude of performance improvement associated with each distinct platform tuning recommendation, thereby allowing the subscriber (e.g., subscribing entity) or user to identify and implement those recommendations that are expected to yield the greatest measurable benefit in improving platform performance.

Each item in the list of platform tuning recommendations, in one or more embodiments, may be selectable and correspond to a distinct platform tuning parameter of the proposed set of platform tuning parameters generated for the target subscriber. In such an embodiment, the system or service implementing method 200 may function to receive, via the graphical user interface, one or more user inputs (e.g., single user input, plurality of user inputs, etc.) selecting at least a subset of the plurality of items included in the list of platform tuning recommendations. For instance, in a non-limiting example, the graphical user interface may function to detect one or more inputs from the subscriber selecting four platform tuning parameters out of a total of sixteen available platform tuning parameters. In another non-limiting example, the graphical user interface may function to detect one or more inputs from the subscriber selecting sixteen platform tuning parameters out of a total of sixteen available platform tuning parameters.

In one or more embodiments, the system or service implementing method 200 may function to automatically adapt an encoding of the digital platform to operate from a current state specified by the at least one corpus of subscriber-specific configuration parameters to an adapted state (e.g., updated state, modified state, etc.) defined by at least a subset or, in some embodiments, the entirety of the proposed set of platform tuning parameters. The adapted state may be defined by only a subset of the proposed set of platform tuning parameters, such as when the subscriber or the system selects less than all of the available platform tuning parameters, or by the entirety of the proposed set of platform tuning parameters when all platform tuning parameters are selected for implementation. In this way, the digital platform supports both partial and complete adaptation of the digital platform.

Accordingly, in one or more embodiments, the graphical user interface may further include a user-selectable control element (e.g., a button, toggle, icon, etc.) that when, selected, functions to automatically implement the proposed set of platform tuning parameters selected by the subscriber or user. For instance, with continued reference to the above non-limiting example, while four of the sixteen platform tuning parameters are selected, the system or service implementing method 200 may further receive an input from the subscriber or user selecting the user-selectable control element. In such a non-limiting example, in response to receiving the input selecting the user-selectable control element, the system may automatically modify the configuration of the digital platform in accordance with the selected platform tuning parameters. It shall be recognized that, in some embodiments, modifying the configuration of the digital platform may involve updating thresholds values, computer-executable instructions, computer-executable rules, or other encoded criteria that controls how the platform (e.g., subscriber-specific instance) performs and/or executes automated operations, semi-automated operations, or manual operations.

In another non-limiting example, if all platform tuning parameters are selected within the list of platform tuning recommendations, the system or service implementing method 200 may, in response to receiving a single input selecting the user-selectable control element, automatically implement the entirety of the proposed set of platform tuning parameters. In such an embodiment, the digital platform may be adapted from its current state defined by the existing (or current) subscriber-specific configuration parameters to a fully adapted state defined by all of the proposed platform tuning parameters, thereby implementing every available platform tuning recommendation in a single platform update.

In one or more embodiments, before receiving the input selecting the user-selectable control element, the graphical user interface may enable the subscriber to assess each surfaced platform tuning parameter and modify (or adjust) the recommended value as needed using one or more input user interface (UI) elements. For instance, in a non-limiting example, the graphical user interface may display a threshold parameter with a current value of 15 for the threshold parameter and a recommended value of 10 for the threshold parameter. The subscriber may adjust the recommended value using a slider or numeric text box to establish (or set) an adjusted value of 12 (instead of 10). Accordingly, in response to receiving the input selecting the user-selectable control element, the system or service implementing method 200 may replace the current value of 15 encoded in the digital platform for the threshold parameter with the adjusted value of 12 for the threshold parameter instead of applying the recommended value of 10, thereby adapting the digital platform in accordance with the subscriber-defined adjustment.

In another embodiment, before receiving the input selecting the user-selectable control element, the graphical user interface may display a computer-executable instruction encoded in the digital platform, such as an automated claim follow-up rule. In this example, the current value encoded in the digital platform may specify transmitting a follow-up message 30 days after a claim denial, and the recommended value predicted by the machine learning model may specify transmitting the follow-up message after 10 days. The subscriber may adjust the recommended value using a numeric input field or rule-editing interface to establish an adjusted value of 15 days (instead of 10). Accordingly, in response to receiving the input selecting the user-selectable control element, the system or service implementing method 200 may replace the current instruction of “30 days” encoded in the digital platform with the adjusted instruction of “15 days” instead of applying the recommended instruction of “10 days,” thereby adapting the digital platform in accordance with the subscriber-defined adjustment.

In another embodiment, before receiving the input selecting the user-selectable control element, the graphical user interface may display a computer-executable instruction encoded in the digital platform, such as a coding rule that governs how transactions are routed for review. For example, the current instruction encoded in the digital platform may specify that all laboratory charges must be routed through coding review. The recommendation predicted by the machine learning model may specify modifying this instruction to instead require coding review only for laboratory charges greater than $50. The subscriber may adjust the recommendation using a rule-editing interface that enables insertion, deletion, or modification of conditional logic. Using this interface, the subscriber may establish an adjusted instruction that requires coding review only for laboratory charges greater than $100. Accordingly, in response to receiving the input selecting the user-selectable control element, the system or service implementing method 200 may replace the current instruction requiring review of all laboratory charges with the adjusted instruction requiring review only of laboratory charges greater than $100, instead of applying the recommended instruction of greater than $50, thereby adapting the digital platform in accordance with the subscriber-defined adjustment.

Stated another way, the graphical user interface may display interactive UI elements that allow the subscriber to interact with each surfaced platform tuning parameter. Through these interactive UI elements, the subscriber may view the current value encoded in the digital platform for a respective parameter, see the recommended value predicted by the machine learning model for the respective parameter, and input an adjustment to the recommended value to create an adjusted value for the respective parameter. Once the adjusted value is received by the graphical user interface, the graphical user interface may temporarily store the adjusted value until the subscriber selects the user-selectable control element. In response to detecting the input selecting the user-selectable control element, the system or service implementing method 200 may replace the current value encoded in the digital platform for the respective parameter with the adjusted value, thereby adapting the platform in accordance with the subscriber-provided modification.

Additionally, or alternatively, in one or more embodiments, the graphical user interface may include a before-and-after user interface object for each respective tuning parameter included in the list of platform tuning recommendations. In such an embodiment, the before-and-after user interface object may include a before state representing the current value encoded in the digital platform together with simulation results generated under that current configuration, and an after state representing the recommended value predicted by the machine learning model together with simulation results generated under the recommended configuration. The before-and-after user interface object may be rendered as a side-by-side panel, a split view, a comparative chart, or another suitable visual construct that compares differences in projected platform performance metrics (e.g., denial rate, collection rate, throughput). In this way, the subscriber is provided with a transparent comparison of the expected performance outcomes for the current configuration versus the machine learning–predicted configuration, thereby aiding decision-making prior to selecting the user-selectable control element to implement the recommendation.

Stated another way, in one or more embodiments, the graphical user interface may generate a graphical before-and-after user interface object that graphically illustrates the difference between the current configuration and the recommended configuration predicted by the machine learning model and/or the ensemble of instance assessment machine learning models. In one or more embodiments, the graphical before-and-after user interface object may include visual indicators such as side-by-side panels, comparative charts, or color-coded highlights that depict the simulation results associated with each state. For example, the object may display a current denial rate metric alongside a projected denial rate metric under the recommended configuration, or a current collection rate metric alongside a projected collection rate metric under the recommended configuration. In this way, the graphical before-and-after user interface object conveys, in a clear visual format, the anticipated impact of implementing the machine learning–predicted tuning parameter, thereby aiding the subscriber in evaluating and deciding whether to apply the recommendation.

Additionally, or alternatively, in one or more embodiments, S250 may function to generate, using the first corpus of simulation findings and the second corpus of simulation findings obtained by S240, a respective parameter change impact artifact for each parameter included in the proposed set of parameters generated by S230. The respective parameter change impact artifact generated for a respective parameter of the proposed set of parameters may graphically or textually explain a performance disparity resulting from a respective subscriber-specific instance processing a test set of digital items when the respective parameter is set to an original value defined by the one or more corpora of subscriber-specific parameters and when the respective parameter is set to a (new) value defined by the proposed set of parameters. In other words, the respective parameter change impact artifact generated for the respective parameter may enable a user to visualize, compare, or otherwise understand how modifying the respective parameter from the original value to the new value defined by the proposed set of parameters affects runtime behavior, performance metrics, and/or computing resource utilization of the subscriber-specific instance when processing the same set of digital items, thereby supporting informed approval, rejection, or modification of the proposed parameter change via the graphical user interface. Stated differently, the respective parameter change impact artifact may present a side-by-side or otherwise comparative representation of execution outcomes produced by different values for the respective parameter, thereby enabling direct comparison of runtime outcomes attributable to modification of the respective parameter, including performance improvements, changes in computing resource utilization, and processing efficiency gains. Accordingly, in such an embodiment, the graphical user interface may function to display the respective parameter change impact artifact generated for each parameter included in the proposed set of parameters.

In one or more embodiments, the proposed set of parameters generated by the ensemble of instance assessment machine learning models may include a first individual parameter recommendation, a second individual parameter recommendation, and a third individual parameter recommendation. The first individual parameter recommendation, in such an embodiment, may specify a first distinct proposed modification to a first setting of the target subscriber-specific instance. The second individual parameter recommendation, in such an embodiment, may specify a second distinct proposed modification to a second setting of the target subscriber-specific instance. The third individual parameter recommendation, in such an embodiment, may specify a third distinct proposed modification to a third setting of the target subscriber-specific instance.

An individual parameter recommendation may refer to a machine learning–generated recommendation to modify a specific workflow, setting, threshold, rule activation control value, computer-executable instruction, or other runtime configuration element of the target subscriber-specific instance in order to improve future runtime behavior or computing performance. In other words, an individual parameter recommendation may specify a proposed new value or state for a respective runtime configuration element (e.g., a workflow, a setting, a threshold, a rule activation control value, a computer-executable instruction, or the like) of the target subscriber-specific instance.

Accordingly, in such an embodiment, the graphical user interface may display the first individual parameter recommendation in association with a first approve selectable user interface element and a first reject selectable user interface element. Additionally, or alternatively, in such an embodiment, the graphical user interface may further display the second individual parameter recommendation in association with a second approve selectable user interface element and a second reject selectable user interface element. Additionally, or alternatively, the graphical user interface may further display the third individual parameter recommendation in association with a third approve selectable user interface element and a third reject selectable user interface element, as shown generally by way of example in FIG. 8. In other words, the graphical user interface may display each individual parameter recommendation as an independently actionable recommendation, thereby enabling a user to selectively approve or reject each proposed modification on a per-parameter basis before the target subscriber-specific instance is adapted according to the proposed set of parameters or a user-modified version thereof. Stated another way, the graphical user interface enables granular user control by allowing independent approval or rejection of each individual parameter recommendation, such that only approved proposed modifications are applied, used, and/or implemented when adapting the target subscriber-specific instance.

For instance, in a non-limiting example, the system or service implementing method 200 may function to receive, via the graphical user interface, a first user input from the user selecting the first approve selectable user interface element, wherein selecting the first approve selectable user interface element indicates approval of the first individual parameter recommendation. Additionally, in such a non-limiting example, the system or service implementing method 200 may function to receive, via the graphical user interface, a second user input from the user selecting the second approve selectable user interface element, wherein selecting the second approve selectable user interface element indicates approval of the second individual parameter recommendation. Additionally, in such a non-limiting example, the system or service implementing method 200 may function to receive, via the graphical user interface, a third user input from the user selecting the third reject selectable user interface element, wherein selecting the third reject selectable user interface element indicates rejection of the third individual parameter recommendation.

As described above, in one or more embodiments, in response to receiving a selection input from the user selecting the tune instance button, the system or service may function to automatically adapt, in real-time or near real-time, the target subscriber-specific instance to an adapted subscriber-specific instance this is configured according to a user-modified version of the proposed set of parameters generated by S230. The user-modified version of the proposed set of parameters, in such an embodiment, may include the first individual parameter recommendation and the second individual parameter recommendation based on receiving the first user input and the second user input. It shall be recognized that the user-modified version of the proposed set of parameters excludes the third individual parameter recommendation based on receiving the third additional user input. It shall be further recognized that, in such an embodiment, the adapted subscriber-specific instance is configured according to the user-modified version of the proposed set of parameters instead of the proposed set of parameters generated by S230 based on detecting that the user rejected at least one individual parameter recommendation (e.g., the third individual parameter recommendation) displayed on the graphical user interface.

Accordingly, in such an embodiment, configuring the adapted subscriber-specific instance according to the user-modified version of the proposed set of parameters may include applying only those individual parameter recommendations that were approved by the user via the graphical user interface and refraining from (or foregoing) implementing those individual parameter recommendations that were rejected by the user. In particular, the adapted subscriber-specific instance may be configured by modifying the first setting of the target subscriber-specific instance in accordance with the first individual parameter recommendation and modifying the second setting of the target subscriber-specific instance in accordance with the second individual parameter recommendation, while maintaining the third setting of the target subscriber-specific instance in its original state based on user rejection of the third individual parameter recommendation. In this way, the adapted subscriber-specific instance reflects a selective, user-approved subset of the proposed set of parameters, thereby ensuring that runtime configuration changes are implemented in accordance with explicit user intent while preserving existing configuration values for rejected recommendations.

Stated differently, in one or more embodiments, the adapted subscriber-specific instance is configured such that only approved proposed setting values replace their corresponding original setting values, while rejected proposed setting values are not implemented and the corresponding original setting values are preserved. For instance, in a non-limiting example, prior to adaptation, the target subscriber-specific instance may include a first setting set to a first setting value, a second setting set to a second setting value, and a third setting set to a third setting value. In such an example, the first individual parameter recommendation may specify a first proposed setting value for the first setting that is different from the first setting value, the second individual parameter recommendation may specify a second proposed setting value for the second setting that is different from the second setting value, and the third individual parameter recommendation may specify a third proposed setting value for the third setting that is different from the third setting value. Accordingly, in response to receiving the selection input from the user selecting the tune instance button, the adapted subscriber-specific instance may be configured by at least setting the first setting to the first proposed setting value, setting the second setting to the second proposed setting value, and leaving the third setting at the third setting value rather than setting the third setting to the third proposed setting value (e.g., not setting the third setting to the third proposed setting value).

In one or more embodiments, a system or service implementing method 200 may function to improve computing performance of a subscriber-specific instance of a multi-tenant cloud-based service. In such an embodiment, the system or service implementing method 200 may function to automatically detect, in real-time using one or more processors, a target subscriber-specific instance of the multi-tenant cloud-based service that has not been tuned during a predetermined time span. In one or more embodiments, in response to detecting that the target subscriber-specific instance has not been tuned during the predetermined time span, the system or service implementing method 200 may obtain, in real-time from one or more computer databases, one or more corpora of subscriber-specific parameters characterizing a runtime behavior of the target subscriber-specific instance, wherein at least a subset of the one or more corpora of subscriber-specific parameters controls a runtime of the target subscriber-specific instance.

In one or more embodiments, in response to obtaining the one or more corpora of subscriber-specific parameters, the system or service implementing method 200 may function to convert the one or more corpora of subscriber-specific parameters into a plurality of distinct model input data objects operably configured for input into an ensemble of instance assessment machine learning models. In one or more embodiments, the system or service implementing method 200 may further function to generate, using the ensemble of instance assessment machine learning models, a proposed set of parameters for controlling a future runtime behavior of the target subscriber-specific instance based on providing the plurality of distinct model input data objects as input to the ensemble of instance assessment machine learning models. Accordingly, in response to generating the proposed set of parameters, the system or service implementing method 200 may function to execute one or more computer-based simulations simulating an operation of the target subscriber-specific instance configured according to the proposed set of parameters.

In one or more embodiments, based on or in response to executing the one or more computer-based simulations, the system or service implementing method 200 may function to display, on a graphical user interface, the proposed set of parameters in association with simulation findings outputted by the one or more computer-based simulations. In such an embodiment, while displaying the graphical user interface, the system or service implementing method 200 may function to receive an input from a user selecting a tune instance button displayed on the graphical user interface. Accordingly, in response to receiving the input from the user selecting the tune instance button, the system or service implementing method 200 may function to automatically execute an instance adaptation process that adapts, in real-time or near real-time, the target subscriber-specific instance to an adapted subscriber-specific instance. It shall be recognized that the adapted subscriber-specific instance may be configured according to the proposed set of parameters or a user-modified version of the proposed set of parameters and/or all future automated digital actions associated with the user that occur within a target time span (e.g., next 3 days, next 7 days, next 30 days, etc.) are executed using the adapted subscriber-specific instance instead of the target subscriber-specific instance.

In one or more embodiments, the ensemble of instance assessment machine learning models may have generated the subset of the one or more corpora of subscriber-specific parameters before the target subscriber-specific instance was adapted to the adapted subscriber-specific instance. Accordingly, in one or more embodiments, the system or service implementing method 200 may adapt an original subscriber-specific instance configured according to a default set of parameters defined by the multi-tenant cloud-based service to the target subscriber-specific instance configured according to the subset of the one or more corpora of subscriber-specific parameters. Accordingly, in such an embodiment, adapting the original subscriber-specific instance to the target subscriber-specific instance may include at least modifying a first distinct automated workflow and a second distinct automated workflow encoded in the original subscriber-specific instance.

Additionally, or alternatively, the system or service implementing method 200 may function to generate an instance change log user interface object. An instance change log user interface object, in one or more embodiments, may refer to a graphical user interface element that presents a structured summary of configuration changes applied to and/or implemented within a subscriber-specific instance during one or more previous (e.g., historical) adaptation processes. Stated another way, an instance change log user interface object, in some embodiments, may refer to a structured graphical user interface element that includes a digital record of configuration changes applied to a subscriber-specific instance, including changes to automated workflows, settings, rules, thresholds, or other runtime configuration elements. It shall be recognized that, in some embodiments, the instance change log user interface object may be configured to display, for each change, identifying information for the affected module or workflow, a description of the modified functionality, a previous configuration state, and a corresponding updated configuration state, thereby enabling a user to review, audit, or understand how the subscriber-specific instance has been adapted over time.

The instance change log user interface object may include, for each modified automated workflow, an identifier of a module in which the automated workflow operates, a text-based description describing a function of the automated workflow, a previous configuration state of the automated workflow, and a corresponding updated configuration state of the automated workflow. In some embodiments, the instance change log user interface object may further include attribution and timing information indicating an identity of a user account or artificial intelligence (AI) agent that authorized a respective modification and a time at which the respective modification occurred. The instance change log user interface object may be displayed within the graphical user interface in association with the proposed set of parameters generated by the ensemble of instance assessment machine learning models and simulation findings outputted by one or more computer-based simulations.

For instance, in a non-limiting example, the system or service implementing method 200 may have adapted the original subscriber-specific instance to the target subscriber-specific instance by at least modifying a first distinct automated workflow and a second distinct automated workflow encoded in the original subscriber-specific instance. In such a non-limiting example, the system or service implementing method 200 may function to generate an instance change log user interface object that may include a first module identifier indicating that the first distinct automated workflow operates within a first distinct module of the multi-tenant cloud-based service, a first text-based description describing a function of the first distinct automated workflow (e.g., “automatically transmit follow-up messages for unresolved claims after a defined time period”), a first previous configuration state specifying a prior configuration state of the first distinct automated workflow (e.g., the first previous configuration state indicates that the automated follow-up workflow was configured to execute after thirty (30) days), a first updated configuration state specifying a new configuration state of the first distinct automated workflow (e.g., the automated follow-up workflow was reconfigured to execute after ten (10) days for high-dollar claims exceeding a threshold amount).

Additionally, in such a non-limiting example, the instance change log user interface may further include a second module identifier indicating that the second distinct automated workflow operates within a second distinct module of the multi-tenant cloud-based service, a second text-based description describing a function of the second distinct automated workflow (e.g., “automatically route denied claims to a coding review queue based on one or more exception conditions”), a second previous configuration state specifying a prior configuration state of the second distinct automated workflow (e.g., the second previous configuration state indicates that all denied claims were automatically routed to the coding review queue regardless of claim amount or exception type), and a second updated configuration state specifying a new configuration state of the second distinct automated workflow (e.g., the automated routing workflow was reconfigured to route only denied claims exceeding a predefined monetary threshold or satisfying one or more exception criteria to the coding review queue, while allowing other denied claims to bypass manual review).

Additionally, in one or more embodiments, the instance change log user interface may further include a first user identifier indicating an identity of a first user account that authorized the modification of the first distinct automated workflow from the first previous configuration state to the first updated configuration state, a first clock time indicating a time at which the first distinct automated workflow was modified from the first previous configuration state to the first updated configuration state, a second user identifier indicating that the first user account authorized the modification of the second distinct automated workflow from the second previous configuration state to the second updated configuration state, and a second clock time indicating a time at which the second distinct automated workflow was modified from the second previous configuration state to the second updated configuration state.

Alternatively, in one or more embodiments, the instance change log user interface may further include a user identifier identifying an artificial intelligence (AI) agent as a user that (i) authorized the modification of the first distinct automated workflow from the first previous configuration state to the first updated configuration state and (ii) authorized the modification of the second distinct automated workflow from the second previous configuration state to the second updated configuration state.

It shall be further recognized that, in some embodiments, the system or service implementing method 200 may function to receive an input from the user selecting the first previous configuration state specifying the prior configuration state of the first distinct automated workflow or the second previous configuration state specifying the prior configuration state of the second distinct automated workflow. Accordingly, in response to receiving the input from the user selecting the first previous configuration state specifying the prior configuration state of the first distinct automated workflow or the second previous configuration state specifying the prior configuration state of the second distinct automated workflow, the system or service implementing method 200 may function to automatically revert (or adapt) a current iteration of the subscriber-specific instance corresponding to the user to a previous iteration of the subscriber-specific instance that is configured according to the selected prior configuration state.

For instance, in a non-limiting example, the current iteration of the subscriber-specific instance may correspond to the target subscriber-specific instance configured according to the subset of the one or more corpora of subscriber-specific parameters. In such a non-limiting example, reverting the current iteration of the subscriber-specific instance to the previous iteration of the subscriber-specific instance may include automatically adapting (or reverting) the target subscriber-specific instance back to the original subscriber-specific instance configured according to the default set of parameters defined by the multi-tenant cloud-based service.

In another non-limiting example, the current iteration of the subscriber-specific instance may correspond to the adapted subscriber-specific instance configured according to the proposed set of parameters or the user-modified version of the proposed set of parameters. In such a non-limiting example, reverting the current iteration of the subscriber-specific instance to the previous iteration of the subscriber-specific instance may include automatically adapting (or reverting) the adapted subscriber-specific instance back to the target subscriber-specific instance configured according to the subset of the one or more corpora of subscriber-specific parameters. In this way, the system or service implementing method 200 enables selective rollback from the adapted subscriber-specific instance to an intermediate configuration state of the subscriber-specific instance, without reverting to the original subscriber-specific instance configured according to the default set of parameters defined by the multi-tenant cloud-based service.

In yet another non-limiting example, the current iteration of the subscriber-specific instance may correspond to the adapted subscriber-specific instance configured according to the proposed set of parameters or the user-modified version of the proposed set of parameters. In such a non-limiting example, reverting the current iteration of the subscriber-specific instance to the previous iteration of the subscriber-specific instance may include automatically adapting (or reverting) the adapted subscriber-specific instance back to the original subscriber-specific instance configured according to the default set of parameters defined by the multi-tenant cloud-based service, thereby undoing all modifications applied when adapting the original subscriber-specific instance to the target subscriber-specific instance and when subsequently adapting the target subscriber-specific instance to the adapted subscriber-specific instance.

### Example Subscriber-Specific Instance Adaptations

In one or more embodiments, a system or service implementing method 200 may function to improve computing performance of a subscriber-specific instance of a multi-tenant cloud-based service. In such an embodiment, the system or service implementing method 200 may function to automatically detect, in real-time using one or more processors, a target subscriber-specific instance of the multi-tenant cloud-based service that has not been tuned during a predetermined time span. In one or more embodiments, in response to detecting that the target subscriber-specific instance has not been tuned during the predetermined time span, the system or service implementing method 200 may obtain, in real-time from one or more computer databases, one or more corpora of subscriber-specific parameters characterizing a runtime behavior of the target subscriber-specific instance, wherein at least a subset of the one or more corpora of subscriber-specific parameters controls a runtime of the target subscriber-specific instance.

In one or more embodiments, in response to obtaining the one or more corpora of subscriber-specific parameters, the system or service implementing method 200 may function to convert the one or more corpora of subscriber-specific parameters into a plurality of distinct model input data objects operably configured for input into an ensemble of instance assessment machine learning models. In one or more embodiments, the system or service implementing method 200 may further function to generate, using the ensemble of instance assessment machine learning models, a proposed set of parameters for controlling a future runtime behavior of the target subscriber-specific instance based on providing the plurality of distinct model input data objects as input to the ensemble of instance assessment machine learning models. Accordingly, in response to generating the proposed set of parameters, the system or service implementing method 200 may function to execute one or more computer-based simulations simulating an operation of the target subscriber-specific instance configured according to the proposed set of parameters.

In one or more embodiments, based on or in response to executing the one or more computer-based simulations, the system or service implementing method 200 may function to display, on a graphical user interface, the proposed set of parameters in association with simulation findings outputted by the one or more computer-based simulations. In such an embodiment, while displaying the graphical user interface, the system or service implementing method 200 may function to receive an input from a user selecting a tune instance button displayed on the graphical user interface. Accordingly, in response to receiving the input from the user selecting the tune instance button, the system or service implementing method 200 may function to automatically execute an instance adaptation process that adapts, in real-time or near real-time, the target subscriber-specific instance to an adapted subscriber-specific instance. It shall be recognized that the adapted subscriber-specific instance may be configured according to the proposed set of parameters or a user-modified version of the proposed set of parameters and/or all future automated digital actions associated with the user that occur within a target time span (e.g., next 3 days, next 7 days, next 30 days, etc.) are executed using the adapted subscriber-specific instance instead of the target subscriber-specific instance.

In a first non-limiting example, the one or more corpora of subscriber-specific parameters may specify a current threshold value for a respective minimum time-based threshold. In such a non-limiting example, the target subscriber-specific instance may be configured to automatically route a subject digital item to a disposal queue when a target time-based attribute associated with the subject digital item is equal to or greater than the current threshold value specified for the respective minimum time-based threshold. Furthermore, in such a non-limiting example, the proposed set of parameters generated by the ensemble of instance assessment machine learning models may specify a new threshold value for the respective minimum time-based threshold. Accordingly, when the target subscriber-specific instance is adapted to the adapted subscriber-specific instance, the adapted subscriber-specific instance may be configured to automatically route the subject digital item to the disposal queue when the target time-based attribute associated with the subject digital item is equal to or greater than the new threshold value specified for the respective minimum time-based threshold, wherein the new threshold value is greater than or less than the current threshold value specified for the respective minimum time-based threshold.

In a second non-limiting example, the one or more corpora of subscriber-specific parameters may specify a current threshold value for a respective maximum threshold. In such a non-limiting example, the target subscriber-specific instance may be configured to automatically route a subject digital item to a disposal queue when a target attribute associated with the subject digital item is less than the current threshold value specified for the respective maximum threshold. Furthermore, in such a non-limiting example, the proposed set of parameters generated by the ensemble of instance assessment machine learning models may specify a new threshold value for the respective maximum threshold. Accordingly, when the target subscriber-specific instance is adapted to the adapted subscriber-specific instance, the adapted subscriber-specific instance may be configured to automatically route the subject digital item to the disposal queue when the target attribute associated with the subject digital item is less than the new threshold value specified for the respective maximum threshold, wherein the new threshold value is greater than or less than the current threshold value specified for the respective maximum threshold.

In a third non-limiting example, the one or more corpora of subscriber-specific parameters may specify a current threshold value for a respective minimum machine learning model prediction threshold. In such a non-limiting example, the target subscriber-specific instance may be configured to automatically predict, using a first set of machine learning models, a probability of a subject digital item having an adverse decision being reversed and, in turn, the target subscriber-specific instance is configured to automatically correct one or more defects in the subject digital item to resolve the adverse decision associated with the subject digital item when the probability is greater than or equal to the current threshold value for the respective minimum machine learning model prediction threshold. Furthermore, in such a non-limiting example, the proposed set of parameters generated by the ensemble of instance assessment machine learning models may specify a new threshold value for the respective minimum machine learning model prediction threshold. Accordingly, when the target subscriber-specific instance is adapted to the adapted subscriber-specific instance, the adapted subscriber-specific instance may be configured to automatically correct the one or more defects in the subject digital item to resolve the adverse decision when the probability is greater than or equal to the new threshold value specified for the respective minimum machine learning model prediction threshold, wherein the new threshold value is greater than or less than the current threshold value specified for the respective minimum machine learning model prediction threshold.

In a fourth non-limiting example, the one or more corpora of subscriber-specific parameters may specify a first computer-executable medical coding instruction that specifies a first set of medical events that require medical coding and a second computer-executable medical coding instruction that specifies a second set of medical events that do not require medical coding. In such a non-limiting example, the target subscriber-specific instance may be configured to automatically assess, by executing the first computer-executable medical coding instruction and the second computer-executable medical coding instruction, one or more medical events included in a subject digital item to determine whether the one or more medical events correspond to the first set of medical events or the second set of medical events and, in turn, automatically generate a digital artifact explaining the one or more medical events performed on a respective entity without any medical codes or coding-related information in response to the target subscriber-specific instance detecting that the one or more medical events corresponds to the second set of medical events. Furthermore, in such a non-limiting example, the proposed set of parameters generated by the ensemble of instance assessment machine learning models may specify a new iteration of the first computer-executable medical coding instruction and a new iteration of the second computer-executable medical coding instruction. It shall be recognized that the new iteration of the first computer-executable medical coding instruction may include one or more additional medical events that require medical coding when compared to the first set of medical events of the first computer-executable medical coding instruction. It shall be further recognized that the new iteration of the second computer-executable medical coding instruction may include a reduced set of medical events that do not require medical coding when compared to the second set of medical events of the second computer-executable medical coding instruction. Accordingly, when the target subscriber-specific instance is adapted to the adapted subscriber-specific instance, the adapted subscriber-specific instance may be configured to automatically assess, by executing the new iteration of the first computer-executable medical coding instruction and the new iteration of the second computer-executable medical coding instruction, the one or more medical events included in the subject digital item to determine whether the one or more medical events correspond to the one or more additional medical events or the reduced set of medical events and, in turn, automatically generate a corresponding digital artifact that explains the one or more medical events performed on the respective entity in association with one or more medical codes in response to the adapted subscriber-specific instance detecting that the one or more medical events corresponds to the one or more additional medical events.

In a fifth non-limiting example, the one or more corpora of subscriber-specific parameters may specify a data acquisition computer procedure that the target subscriber-specific instance invokes to periodically fetch data from one or more electronic health record systems. The system or service implementing method 200 may function to detect, using the ensemble of instance assessment machine learning models, an anomaly or misconfiguration in the data acquisition computer procedure. Furthermore, in such a non-limiting example, the proposed set of parameters generated by the ensemble of instance assessment machine learning models may specify a new data acquisition computer procedure that corrects the anomaly or misconfiguration detected in the data acquisition computer procedure. Accordingly, when the target subscriber-specific instance is adapted to the adapted subscriber-specific instance, the adapted subscriber-specific instance is configured to automatically invoke the new data acquisition computer procedure when performing subsequent data acquisition operations to retrieve new data from the one or more electronic health record systems during the target time span.

It shall be recognized that, in one or more embodiments, the anomaly or misconfiguration detected in the data acquisition computer procedure may include at least a missing or incomplete parameter required for periodically fetching the data from the one or more electronic health record systems and, in turn, the new data acquisition computer procedure may include a parameter value for the missing or incomplete parameter.

In a sixth non-limiting example, the one or more corpora of subscriber-specific parameters may specify a rule activation control value for a respective computer-executable rule (e.g., the respective computer-executable rule is configured to automatically perform, using the one or more processors, an automated operation when a digital item satisfies one or more logical conditions included in the respective computer-executable rule). The rule activation control value, in such an embodiment, may be set to a first value (e.g., 0, false, or the like) in the one or more corpora of subscriber-specific parameters. In one or more embodiments, the respective computer-executable rule is deactivated in the target subscriber-specific instance based on the rule activation control value for the respective computer-executable rule being set to the first value in the one or more corpora of subscriber-specific parameters. Furthermore, in such a non-limiting example, the proposed set of parameters generated by the ensemble of instance assessment machine learning models may recommend the rule activation control value for the respective computer-executable rule be set to a second value (e.g., 1, true, or the like) different from the first value. Accordingly, when the target subscriber-specific instance is adapted to the adapted subscriber-specific instance, the respective computer-executable rule is activated in the adapted subscriber-specific instance based on the rule activation control value for the respective computer-executable rule being set to the second value.

In a seventh non-limiting example, the one or more corpora of subscriber-specific parameters may specify a rule activation control value for a respective computer-executable rule (e.g., the respective computer-executable rule is configured to automatically perform, using the one or more processors, an automated operation when a digital item satisfies at least two logical conditions included in the respective computer-executable rule). The rule activation control value, in one or more embodiments, may be set to a first binary value in the one or more corpora of subscriber-specific parameters. In one or more embodiments, the target subscriber-specific instance does not execute or use the respective computer-executable rule based on the rule activation control value for the respective computer-executable rule being set to the first binary value. Furthermore, in such a non-limiting example, the proposed set of parameters generated by the ensemble of instance assessment machine learning models may specify that the rule activation control value for the respective computer-executable rule is to be set to a second binary value different from the first binary value. Accordingly, when the target subscriber-specific instance is adapted to the adapted subscriber-specific instance, the adapted subscriber-specific instance executes or uses the respective computer-executable rule based on the rule activation control value for the respective computer-executable rule being set to the second binary value.

In an eighth non-limiting example, the ensemble of instance assessment machine learning models may assess a pending digital item (e.g., digital claim, digital item, or the like) stored within the target subscriber-specific instance against a set of computer-executable rules (e.g., National Correct Coding Initiative (NCCI) edits, local and/or national coverage determination (LCI/NCD) rules, medically unlikely edit (MUE) validation rules, or any suitable combinations thereof). In such a non-limiting example, the proposed set of parameters generated by the ensemble of instance assessment machine learning models may specify or recommend changing at least one target data attribute in the pending digital item from a first data value (as currently represented in the pending digital item) to a second data value different from the first data value. Accordingly, in one or more embodiments, the system or service implementing method 200 may function to receive, via a graphical user interface, a chatbot interface, or the like, an input from a user associated with accepting (e.g., approving or the like) the recommended change for the pending digital item. Accordingly, in one or more embodiments, in response to receiving the input from the user, the system or service implementing method 200 may function to replace the first data value encoded in the pending digital claim with the second data value.

Furthermore, in response to receiving the input from the user associated with accepting the recommended change for the pending digital item, the system or service implementing method 200 may function to automatically assess a plurality of additional pending digital items (e.g., 1,000 additional pending digital items, 1,000,000 additional pending digital items, etc.) stored in a digital item queue and, in turn, detect that at least a subset (e.g., 10 additional pending digital claims, 500 additional pending digital items, etc.) of the plurality of additional pending digital items have the at least one target data attribute set to the first data value. In one or more embodiments, in response to detecting that the subset of additional pending digital items have the at least one target data attribute set to the first data value, the system or service implementing method 200 may function to generate, via a graphical user interface, a chatbot interface, or the like, a notification requesting confirmation to apply the recommended change specified in the proposed set of parameters to the subset. In such an embodiment, the graphical user interface, the chatbot interface, or the like may surface a selectable control user interface element operably configured to receive a user input authorizing simultaneous and bulk replacement of the first data value with the second data value across the subset of additional pending digital items, as shown generally by way of example in FIGS. 9-25 .

Accordingly, in one or more embodiments, the system or service implementing method 200 may function to receive the user input selecting the selectable control user interface element and, in response to receiving the user input, the system or service implementing method 200 may function to simultaneously replace, in bulk, the first data value attributed to the at least one target data attribute within each additional pending digital item of the subset with the second data value specified in the proposed set of parameters. Stated another way, in one or more embodiments, the system or service implementing method 200 may automatically and/or simultaneously propagate, in bulk, the user-approved change identified for the pending digital item across the subset of additional pending digital items in a coordinated, computer-implemented operation prior to submission of the subset of additional pending digital items to an external adjudication or processing system. At least one technical benefit of such an embodiment includes reducing a total number of invocations of the ensemble of instance assessment machine learning models by preventing repeated detection of a same defect, error, and/or anomaly.

In one or more embodiments, the system or service may provide the proposed set of parameters and corresponding recommended actions through a conversational interface (e.g., chatbot interface). The chatbot interface may be configured to present an explanation of a detected defect, error, and/or anomaly, present an estimated likelihood of an adverse downstream event (e.g., rejection or other processing failure), and present an ordered set of corrective actions for modifying one or more data attributes, workflow parameters, and/or rule activation control values. In some embodiments, the chatbot interface may further be configured to execute one or more corrective actions upon receiving user approval and to generate messages reflecting an execution status of the corrective actions. In one or more embodiments, in response to receiving an approval input associated with a recommended change for a pending digital item, the system or service may assess a digital item queue to detect a subset of additional pending digital items exhibiting a same or similar defect signature, anomaly signature, and/or rule-activation pattern. The system or service may surface, via the graphical user interface and/or chatbot interface, a selectable control configured to receive a user input authorizing application of the recommended change across the detected subset. Upon receiving the user input, the system or service may simultaneously and in bulk apply the recommended change across the detected subset, including replacing a first data value for at least one target data attribute with a second data value, and/or updating a rule activation control value for a respective computer-executable rule.

In one or more embodiments, the system or service implementing method 200 may function to implement a multi-stage feedback loop configured to improve predictive accuracy, recommendation quality, and computational efficiency of the ensemble of instance assessment machine learning models operating on a target subscriber-specific instance.

In a first stage of the multi-stage feedback loop, the system or service may generate outcome-based feedback signals independent of explicit user input. In such embodiments, the system or service may monitor downstream outcomes associated with previously generated predictions and recommended parameter changes, including whether a predicted adverse event, defect, error, and/or anomaly was subsequently observed, avoided, or resolved following implementation of a recommended change. The system or service may compare the monitored downstream outcomes to corresponding predicted outcomes generated by the ensemble of instance assessment machine learning models and generate one or more accuracy signals indicative of prediction correctness, confidence calibration, or recommendation effectiveness. Based at least in part on the accuracy signals, the system or service may update one or more auxiliary data structures associated with the ensemble of instance assessment machine learning models, such as a knowledge graph, clustering structure, pattern repository, or feature-importance map.

For example, when a recommended change associated with a particular defect signature consistently results in resolution of the defect across multiple pending digital items, the system or service may strengthen or increase an association between the defect signature and the recommended corrective action (e.g., proposed parameter or the like) within the auxiliary data structures. Conversely, when a recommended change fails to resolve a defect or produces an unexpected outcome, the system or service may weaken, decrease, modify, or remove a corresponding association. In some embodiments, the first-stage feedback loop may be utilized during periodic retraining, recalibration, or re-weighting of one or more models of the ensemble of instance assessment machine learning models.

In another example of the first-stage feedback loop, the system or service may detect that multiple pending digital items exhibiting a similar combination of input attributes are repeatedly associated with a same downstream outcome, despite having been assigned to different clusters within a clustering structure or knowledge graph. In response, the system or service may merge the clusters, update one or more centroid representations, and adjust feature weights associated with the merged cluster to reflect a newly identified operational pattern.

In another example, the system or service may determine that a previously established cluster representing a defect, error, and/or anomaly exhibits divergent downstream outcomes across different subsets of pending digital items. In such embodiments, the system or service may split the cluster into two or more sub-clusters based on detected differences in input attributes, contextual parameters, or rule-activation control values, thereby enabling more granular pattern representation and improving subsequent prediction accuracy.

In yet another example, the system or service may identify that a cluster associated with a previously frequent defect, error, and/or anomaly has not been observed within a threshold time period or across a threshold number of recently processed pending digital items. In response, the system or service may reduce a confidence weight associated with the cluster, mark the cluster as dormant, or remove the cluster from the knowledge graph or pattern repository, thereby reducing unnecessary evaluation of stale or obsolete patterns during subsequent assessments.

In one or more embodiments, the system or service may generate recommendations, including a proposed set of parameters, for a pending digital item based at least in part on a cluster to which the pending digital item is assigned within the clustering structure or knowledge graph. In such embodiments, assignment of the pending digital item to a particular cluster may be based on similarity between one or more input attributes of the pending digital item and a centroid, pattern signature, or feature representation associated with the cluster. The proposed set of parameters may be derived from one or more corrective actions, parameter values, or rule-activation control values that are statistically associated with favorable downstream outcomes for other digital items previously assigned to the same cluster. For example, when a pending digital item is mapped to a cluster representing a recurring defect that has historically been resolved by modifying a particular target data attribute from a first data value to a second data value, the system or service may include such modification in the proposed set of parameters. In this manner, the clustering structure or knowledge graph may function as a decision-support substrate (e.g., tool) that guides generation, prioritization, and confidence scoring of recommendations produced by the ensemble of instance assessment machine learning models. Stated another way, the system or service may leverage historical outcome-validated patterns encoded in the clustering structure or knowledge graph to automatically infer and recommend parameter changes for a pending digital item that is determined to be similar or equivalent to previously assessed digital items.

Additionally, or alternatively, in one or more embodiments, a second stage of the multi-stage feedback loop may use explicit user feedback and prompt-based critique operations performed by one or more language models. In such embodiments, the system or service may receive explicit user feedback via a graphical user interface, a chatbot interface, or the like, including a recommendation acceptance input, a recommendation rejection input (e.g., a downvote), a confidence indicator, and/or a user-provided annotation associated with a previously generated recommendation or proposed set of parameters. In one or more embodiments, in response to receiving a rejection signal (e.g., recommendation rejection input, etc.) or negative explicit user feedback, the system or service may initiate a prompt-based critique operation by constructing a structured prompt for a language model. The structured prompt may include contextual data associated with the rejected recommendation, including one or more of: (i) underlying input attributes of the pending digital item, (ii) an original analysis generated by the ensemble of instance assessment machine learning models for the pending digital item, (iii) an original proposed set of parameters or corrective action sequence generated for the pending digital item, and (iv) any user-provided annotation or comment explaining a rationale for the rejection. The structured prompt may further include an instruction explicitly identifying the original recommendation (e.g., original proposed set of parameters or corrective action sequence) as erroneous or suboptimal and requesting the language model to critique the original analysis and generate an improved analysis and/or revised proposed set of parameters.

For example, when a user provides a rejection signal in the form of a downvote for a recommendation associated with a pending digital item and includes a comment indicating that a recommended modifier, parameter value, or rule-activation control value for the pending digital item is incorrect, the system or service may package the underlying data of the pending digital item, the original analysis, the original corrective action sequence, and the user-provided comment into the structured prompt. The prompt may instruct the language model that the recommendation for the pending digital item is an error and request that the language model critique the reasoning underlying the recommendation and generate an improved corrective action sequence (e.g., new set of proposed parameters) for the pending digital item. In another example, when a user rejects a recommendation generated for a pending digital item without providing an explicit annotation, the system or service may generate a structured prompt instructing the language model to infer potential causes of the rejection based on attributes of the pending digital item and differences between the pending digital item and historical digital items associated with favorable outcomes within a corresponding cluster. The language model may be prompted to generate a revised proposed set of parameters for the pending digital item that accounts for the inferred differences. In yet another example, when a user partially accepts a recommendation for a pending digital item by modifying one or more elements of the proposed set of parameters prior to approval, the system or service may generate a structured prompt instructing the language model to compare the originally proposed parameters for the pending digital item to the user-modified parameters, critique the original recommendation in view of the modification, and generate an updated recommendation strategy for the pending digital item. It shall be recognized that revised analyses, revised proposed sets of parameters, and/or revised corrective action sequences generated via the prompt-based critique operations for the pending digital item may be persistently stored and associated with a corresponding cluster, pattern signature, or rule-activation context. The stored revisions may be utilized by the ensemble of instance assessment machine learning models during subsequent assessments of other pending digital items that are determined to be similar or equivalent to the pending digital item.

In one or more embodiments, the second-stage feedback loop cooperates with the first-stage feedback loop such that outcome-based accuracy signals and explicit user-driven prompt-based revisions associated with the pending digital item inform future predictions by the ensemble of instance assessment machine learning models. As a result, when subsequently assessed pending digital items exhibit attributes similar or equivalent to those of the pending digital item, the system or service may avoid repeating erroneous or suboptimal analyses and instead generate recommendations informed by stored, outcome-validated, and user-corrected scenarios.

At least one technical benefit of the multi-stage feedback loop includes improved predictive accuracy over time, reduced propagation of repeated defects, errors, and/or anomalies across pending digital items, reduced repeated invocation of the ensemble of instance assessment machine learning models and language models for substantially similar conditions, accelerated convergence toward accurate recommendations, and improved computational efficiency and stability of the system or service as operational patterns evolve within the target subscriber-specific instance.

It shall also be noted that the system and methods of the embodiments and variations described herein can be embodied and/or implemented at least in part as a machine comprising a computer-readable medium storing computer-readable instructions. The instructions may be executed by computer-executable components integrated with the system and one or more portions of the processors and/or the controllers. The computer-readable medium can be stored on any suitable computer-readable media such as RAMs, ROMs, flash memory, EEPROMs, optical devices (CD or DVD), hard drives, floppy drives, memory sticks (e.g., SD cards, USB flash drives), cloud-based services (e.g., cloud storage), magnetic storage devices, Solid-State Drives (SSDs), or any suitable device. The computer-executable component is preferably a general or application-specific processor, but any suitable dedicated hardware or hardware/firmware combination device can alternatively or additionally execute the instructions.

It shall be noted that, in the method(s) described herein where one or more steps (e.g., processes) are contingent upon one or more conditions having been met, it should be understood that the described method can be repeated in multiple repetitions so that over the course of the repetitions all of the conditions upon which steps in the method are contingent have been met in different repetitions of the method. For example, if a method requires performing a first step if a condition is satisfied, and a second step if the condition is not satisfied, then a person of ordinary skill would appreciate that the claimed steps are repeated until the condition has been both satisfied and not satisfied, in no particular order. Thus, a method described with one or more steps that are contingent upon one or more conditions having been met could be rewritten as a method that is repeated until each of the conditions described in the method has been met. This, however, is not required of system or computer readable medium claims where the system or computer readable medium contains instructions for performing the contingent operations based on the satisfaction of the corresponding one or more conditions and thus is capable of determining whether the contingency has or has not been satisfied without explicitly repeating steps of a method until all of the conditions upon which steps in the method are contingent have been met. A person having ordinary skill in the art would also understand that, similar to a method with contingent steps, a system or computer readable storage medium can repeat the steps of a method as many times as are needed to ensure that all of the contingent steps have been performed.

The systems and methods of the preferred embodiments may additionally, or alternatively, be implemented on an integrated software application and/or software architecture such as those offered by Technology Partners LLC d/b/a ImagineSoftware.

Although omitted for conciseness, the preferred embodiments include every combination and permutation of the implementations of the systems and methods described herein in real-time or near real-time, asynchronously (e.g., sequentially), concurrently (e.g., in parallel), or in any other suitable order by and/or using one or more instances of the systems, elements, and/or entities described herein. It shall be noted that “real-time” or “near real-time” as generally used herein may refer to generating an output or performing an action within strict time constraints. For example, in one or more embodiments, real-time may be understood to be instantaneous, on the order of milliseconds, or on the order of minutes. Of course, depending on the particular temporal nature of the system in which an embodiment is implemented, other appropriate timescales may be considered acceptable for real-time or near real-time processing.

Embodiments of the system and/or method can include every combination and permutation of the various system components and the various method processes, wherein one or more instances of the method and/or processes described herein can be performed in real-time or near real-time, asynchronously (e.g., sequentially), concurrently (e.g., in parallel), or in any other suitable order by and/or using one or more instances of the systems, elements, and/or entities described herein.

As a person skilled in the art will recognize from the previous detailed description and from the figures and claims, modifications and changes can be made to the embodiments of the application without departing from the scope of the various described embodiments.

We claim:

1. A computer-implemented method for improving a computing performance of a subscriber-specific instance of a multi-tenant cloud-based service, the computer-implemented method comprising:
automatically detecting, in real-time using one or more processors, a target subscriber-specific instance of the multi-tenant cloud-based service that has not been tuned during a predetermined time span;
in response to detecting that the target subscriber-specific instance has not been tuned during the predetermined time span, obtaining, in real-time from one or more computer databases, one or more corpora of subscriber-specific parameters characterizing a runtime behavior of the target subscriber-specific instance, wherein:
at least a subset of the one or more corpora of subscriber-specific parameters controls a runtime of the target subscriber-specific instance;
converting, in response to obtaining the one or more corpora of subscriber-specific parameters, the one or more corpora of subscriber-specific parameters into a plurality of distinct model input data objects operably configured for input into an ensemble of instance assessment machine learning models;
generating, using the ensemble of instance assessment machine learning models, a proposed set of parameters for controlling a future runtime behavior of the target subscriber-specific instance based on providing the plurality of distinct model input data objects as input to the ensemble of instance assessment machine learning models;
executing, in response to generating the proposed set of parameters, one or more computer-based simulations simulating an operation of the target subscriber-specific instance configured according to the proposed set of parameters;
in response to executing the one or more computer-based simulations, displaying, on a graphical user interface, the proposed set of parameters in association with simulation findings outputted by the one or more computer-based simulations;
receiving, while displaying the graphical user interface, an input from a user selecting a tune instance button displayed on the graphical user interface; and
in response to receiving the input from the user selecting the tune instance button, automatically executing an instance adaptation process that adapts, in real-time or near real-time, the target subscriber-specific instance to an adapted subscriber-specific instance, wherein:
the adapted subscriber-specific instance is configured according to the proposed set of parameters or a user-modified version of the proposed set of parameters, and
all future automated digital actions associated with the user that occur within a target time span are executed using the adapted subscriber-specific instance instead of the target subscriber-specific instance.

2. The computer-implemented method according to claim 1, wherein:
the one or more corpora of subscriber-specific parameters specifies a current threshold value for a respective minimum time-based threshold,
the target subscriber-specific instance is configured to automatically route a subject digital item to a disposal queue when a target time-based attribute associated with the subject digital item is equal to or greater than the current threshold value specified for the respective minimum time-based threshold,
the proposed set of parameters generated by the ensemble of instance assessment machine learning models specifies a new threshold value for the respective minimum time-based threshold, and
the adapted subscriber-specific instance is configured to automatically route the subject digital item to the disposal queue when the target time-based attribute associated with the subject digital item is equal to or greater than the new threshold value specified for the respective minimum time-based threshold, wherein the new threshold value is greater than or less than the current threshold value specified for the respective minimum time-based threshold.

3. The computer-implemented method according to claim 1, wherein:
the one or more corpora of subscriber-specific parameters specifies a current threshold value for a respective maximum threshold,
the target subscriber-specific instance is configured to automatically route a subject digital item to a disposal queue when a target attribute associated with the subject digital item is less than the current threshold value specified for the respective maximum threshold,
the proposed set of parameters generated by the ensemble of instance assessment machine learning models specifies a new threshold value for the respective maximum threshold, and
the adapted subscriber-specific instance is configured to automatically route the subject digital item to the disposal queue when the target attribute associated with the subject digital item is less than the new threshold value specified for the respective maximum threshold, wherein the new threshold value is greater than or less than the current threshold value specified for the respective maximum threshold.

4. The computer-implemented method according to claim 1, wherein:
the one or more corpora of subscriber-specific parameters specifies a current threshold value for a respective minimum machine learning model prediction threshold,
the target subscriber-specific instance is configured to automatically predict, using a first set of machine learning models, a probability of a subject digital item having an adverse decision being reversed,
the target subscriber-specific instance is configured to automatically correct one or more defects in the subject digital item to resolve the adverse decision associated with the subject digital item when the probability is greater than or equal to the current threshold value for the respective minimum machine learning model prediction threshold,
the proposed set of parameters generated by the ensemble of instance assessment machine learning models specifies a new threshold value for the respective minimum machine learning model prediction threshold, and
the adapted subscriber-specific instance is configured to automatically correct the one or more defects in the subject digital item to resolve the adverse decision when the probability is greater than or equal to the new threshold value specified for the respective minimum machine learning model prediction threshold, wherein the new threshold value is greater than or less than the current threshold value specified for the respective minimum machine learning model prediction threshold.

5. The computer-implemented method according to claim 1, wherein:
the one or more corpora of subscriber-specific parameters specifies:
a first computer-executable medical coding instruction that specifies a first set of medical events that require medical coding, and
a second computer-executable medical coding instruction that specifies a second set of medical events that do not require medical coding,
the target subscriber-specific instance is configured to automatically assess, by executing the first computer-executable medical coding instruction and the second computer-executable medical coding instruction, one or more medical events included in a subject digital item to determine whether the one or more medical events correspond to the first set of medical events or the second set of medical events,
the target subscriber-specific instance automatically generates a digital artifact explaining the one or more medical events performed on a respective entity without any medical codes or coding-related information in response to the target subscriber-specific instance detecting that the one or more medical events corresponds to the second set of medical events,
the proposed set of parameters generated by the ensemble of instance assessment machine learning models specifies a new iteration of the first computer-executable medical coding instruction and a new iteration of the second computer-executable medical coding instruction,
the new iteration of the first computer-executable medical coding instruction includes one or more additional medical events that require medical coding when compared to the first set of medical events of the first computer-executable medical coding instruction,
the new iteration of the second computer-executable medical coding instruction includes a reduced set of medical events that do not require medical coding when compared to the second set of medical events of the second computer-executable medical coding instruction,
the adapted subscriber-specific instance is configured to automatically assess, by executing the new iteration of the first computer-executable medical coding instruction and the new iteration of the second computer-executable medical coding instruction, the one or more medical events included in the subject digital item to determine whether the one or more medical events correspond to the one or more additional medical events or the reduced set of medical events, and
the adapted subscriber-specific instance automatically generates a corresponding digital artifact that explains the one or more medical events performed on the respective entity in association with one or more medical codes in response to the adapted subscriber-specific instance detecting that the one or more medical events corresponds to the one or more additional medical events.

6. The computer-implemented method according to claim 1, wherein:
the one or more corpora of subscriber-specific parameters specifies a data acquisition computer procedure that the target subscriber-specific instance invokes to periodically fetch data from one or more electronic health record systems,
the computer-implemented method further comprises detecting, using the ensemble of instance assessment machine learning models, an anomaly or misconfiguration in the data acquisition computer procedure,
the proposed set of parameters generated by the ensemble of instance assessment machine learning models specifies a new data acquisition computer procedure that corrects the anomaly or misconfiguration detected in the data acquisition computer procedure, and
the adapted subscriber-specific instance is configured to automatically invoke the new data acquisition computer procedure when performing subsequent data acquisition operations to retrieve new data from the one or more electronic health record systems during the target time span.

7. The computer-implemented method according to claim 6, wherein:
the anomaly or misconfiguration detected in the data acquisition computer procedure includes at least a missing or incomplete parameter required for periodically fetching the data from the one or more electronic health record systems, and
the new data acquisition computer procedure includes a parameter value for the missing or incomplete parameter.

8. The computer-implemented method according to claim 1, wherein:
the one or more corpora of subscriber-specific parameters specifies a rule activation control value for a respective computer-executable rule,
the rule activation control value is set to a first value in the one or more corpora of subscriber-specific parameters,
the respective computer-executable rule is configured to automatically perform, using the one or more processors, an automated operation when a digital item satisfies one or more logical conditions included in the respective computer-executable rule,
the respective computer-executable rule is deactivated in the target subscriber-specific instance based on the rule activation control value for the respective computer-executable rule being set to the first value in the one or more corpora of subscriber-specific parameters,
the proposed set of parameters generated by the ensemble of instance assessment machine learning models recommends the rule activation control value for the respective computer-executable rule be set to a second value different from the first value, and
the respective computer-executable rule is activated in the adapted subscriber-specific instance based on the rule activation control value for the respective computer-executable rule being set to the second value.

9. The computer-implemented method according to claim 1, wherein:
the one or more corpora of subscriber-specific parameters specifies a rule activation control value for a respective computer-executable rule,
the rule activation control value is set to a first binary value in the one or more corpora of subscriber-specific parameters,
the respective computer-executable rule is configured to automatically perform, using the one or more processors, an automated operation when a digital item satisfies at least two logical conditions included in the respective computer-executable rule,
the target subscriber-specific instance does not execute or use the respective computer-executable rule based on the rule activation control value for the respective computer-executable rule being set to the first binary value,
the proposed set of parameters generated by the ensemble of instance assessment machine learning models specifies that the rule activation control value for the respective computer-executable rule is to be set to a second binary value different from the first binary value, and
the adapted subscriber-specific instance executes or uses the respective computer-executable rule based on the rule activation control value for the respective computer-executable rule being set to the second binary value.

10. The computer-implemented method according to claim 1, wherein:
the ensemble of instance assessment machine learning models generated the subset of the one or more corpora of subscriber-specific parameters before the target subscriber-specific instance was adapted to the adapted subscriber-specific instance, and
the computer-implemented method further includes:
adapting an original subscriber-specific instance configured according to a default set of parameters defined by the multi-tenant cloud-based service to the target subscriber-specific instance configured according to the subset of the one or more corpora of subscriber-specific parameters, wherein adapting the original subscriber-specific instance to the target subscriber-specific instance includes modifying at least a first distinct automated workflow and a second distinct automated workflow encoded in the original subscriber-specific instance;
generating an instance change log user interface object that includes:
a first module identifier indicating that the first distinct automated workflow operates within a first distinct module of the multi-tenant cloud-based service,
a first text-based description describing a function of the first distinct automated workflow,
a first previous configuration state specifying a prior configuration state of the first distinct automated workflow,
a first updated configuration state specifying a new configuration state of the first distinct automated workflow,
a second module identifier indicating that the second distinct automated workflow operates within a second distinct module of the multi-tenant cloud-based service,
a second text-based description describing a function of the second distinct automated workflow,
a second previous configuration state specifying a prior configuration state of the second distinct automated workflow, and
a second updated configuration state specifying a new configuration state of the second distinct automated workflow; and
displaying, within the graphical user interface, the instance change log user interface object in association with the proposed set of parameters generated by the ensemble of instance assessment machine learning models and the simulation findings outputted by the one or more computer-based simulations.

11. The computer-implemented method according to claim 10, wherein the instance change log user interface object displayed on the graphical user interface further includes:
a first user identifier indicating an identity of a first user account that authorized the modification of the first distinct automated workflow from the first previous configuration state to the first updated configuration state,
a first clock time indicating a time at which the first distinct automated workflow was modified from the first previous configuration state to the first updated configuration state,
a second user identifier indicating that the first user account authorized the modification of the second distinct automated workflow from the second previous configuration state to the second updated configuration state, and
a second clock time indicating a time at which the second distinct automated workflow was modified from the second previous configuration state to the second updated configuration state.

12. The computer-implemented method according to claim 10, wherein the instance change log user interface object displayed on the graphical user interface further includes:
a user identifier identifying an artificial intelligence (AI) agent as a user that (i) authorized the modification of the first distinct automated workflow from the first previous configuration state to the first updated configuration state and (ii) authorized the modification of the second distinct automated workflow from the second previous configuration state to the second updated configuration state.

13. The computer-implemented method according to claim 1, wherein:
the proposed set of parameters generated by the ensemble of instance assessment machine learning models includes a first individual parameter recommendation, a second individual parameter recommendation, and a third individual parameter recommendation,
the first individual parameter recommendation specifies a first distinct proposed modification to a first setting of the target subscriber-specific instance,
the second individual parameter recommendation specifies a second distinct proposed modification to a second setting of the target subscriber-specific instance,
the third individual parameter recommendation specifies a third distinct proposed modification to a third setting of the target subscriber-specific instance,
the graphical user interface displays the first individual parameter recommendation in association with a first approve selectable user interface element and a first reject selectable user interface element,
the graphical user interface displays the second individual parameter recommendation in association with a second approve selectable user interface element and a second reject selectable user interface element, and
the graphical user interface displays the third individual parameter recommendation in association with a third approve selectable user interface element and a third reject selectable user interface element.

14. The computer-implemented method according to claim 13, further comprising:
receiving, via the graphical user interface, a first additional user input from the user selecting the first approve selectable user interface element, wherein selecting the first approve selectable user interface element indicates approval of the first individual parameter recommendation;
receiving, via the graphical user interface, a second additional user input from the user selecting the second approve selectable user interface element, wherein selecting the second approve selectable user interface element indicates approval of the second individual parameter recommendation;
receiving, via the graphical user interface, a third additional user input from the user selecting the third reject selectable user interface element, wherein selecting the third reject selectable user interface element indicates rejection of the third individual parameter recommendation, wherein:
the user-modified version of the proposed set of parameters includes the first individual parameter recommendation and the second individual parameter recommendation based on receiving the first additional user input and the second additional user input,
the user-modified version of the proposed set of parameters excludes the third individual parameter recommendation based on receiving the third additional user input, and
the adapted subscriber-specific instance is configured according to the user-modified version of the proposed set of parameters based on detecting the user rejected the third individual parameter recommendation.

15. The computer-implemented method according to claim 14, wherein:
the first setting is set to a first setting value within the target subscriber-specific instance,
the second setting is set to a second setting value within the target subscriber-specific instance,
the third setting is set to a third setting value within the target subscriber-specific instance,
the first distinct proposed modification of the first individual parameter recommendation specifies a first proposed setting value for the first setting that is different from the first setting value,
the second distinct proposed modification of the second individual parameter recommendation specifies a second proposed setting value for the second setting that is different from the second setting value,
the third distinct proposed modification of the third individual parameter recommendation specifies a third proposed setting value for the third setting that is different from the third setting value, and
configuring the adapted subscriber-specific instance according to the user-modified version of the proposed set of parameters includes setting:
(i) the first setting of the adapted subscriber-specific instance to the first proposed setting value,
(ii) the second setting of the adapted subscriber-specific instance to the second proposed setting value, and
(ii) the third setting of the adapted subscriber-specific instance to the third setting value instead of the third proposed setting value.

16. The computer-implemented method according to claim 1, wherein:
the ensemble of instance assessment machine learning models generates the proposed set of parameters based on assessing the plurality of distinct model input data objects against:
(i) historical claim and payment data associated with the target subscriber-specific instance of the multi-tenant cloud-based service,
(ii) a plurality of distinct payer contract data objects that each specify one or more payer-specific rules, one or more payer-specific reimbursement policies, and one or more payer-specific compliance requirements applicable to digital items processed by the target subscriber-specific instance,
(iii) machine-readable industry standard specification data obtained from one or more external data sources,
(iv) one or more machine-readable industry standard rule sets obtained from the one or more external data sources, and
(v) historical benchmark data of a subscribing entity that corresponds to the target subscriber-specific instance.

17. The computer-implemented method according to claim 1, wherein:
the target subscriber-specific instance corresponds to a subscribing entity subscribing to the multi-tenant cloud-based service, and
the computer-implemented method further includes:
detecting, using the one or more processors, that a difference between an efficacy metric value computed for a subject efficacy metric during the target time span and a benchmark efficacy value defined for the subject efficacy metric exceeds a predefined deviation threshold,
in response to detecting that the difference exceeds the predefined deviation threshold, generating, in real-time, an alert indicating the adapted subscriber-specific instance is behaving anomalously with respect to the subject efficacy metric, and
transmitting, in real-time over a computer network, the alert to the subscribing entity.

18. The computer-implemented method according to claim 1, further comprising:
executing an additional computer-based simulation simulating operation of the target subscriber-specific instance configured according to the subset of the one or more corpora of subscriber-specific parameters, wherein executing the additional computer-based simulation includes:
providing a test set of digital items to the target subscriber-specific instance configured according to the subset of the one or more corpora of subscriber-specific parameters, and
computing a first corpus of simulation findings based on processing the test set of digital items using the target subscriber-specific instance configured according to the subset of the one or more corpora of subscriber-specific parameters.

19. The computer-implemented method according to claim 18, wherein:
executing the one or more computer-based simulations simulating the operation of the target subscriber-specific instance configured according to the proposed set of parameters includes:
providing the test set of digital items to the target subscriber- specific instance configured according to the proposed set of parameters, and
computing a second corpus of simulation findings based on processing the test set of digital items using the target subscriber-specific instance configured according to the proposed set of parameters, wherein the simulation findings outputted by the one or more computer-based simulations includes the second corpus of simulation findings.

20. The computer-implemented method according to claim 19, further comprising:
generating, using the first corpus of simulation findings and the second corpus of simulation findings, a respective parameter change impact artifact for each parameter included in the proposed set of parameters, wherein:
the respective parameter change impact artifact generated for a respective parameter of the proposed set of parameters graphically or textually explains a performance disparity resulting from processing the test set of digital items using (i) an original parameter value defined by the subset of the one or more corpora of subscriber-specific parameters for the respective parameter and (ii) a modified parameter value defined by the proposed set of parameters for the respective parameter, and
the graphical user interface displays the respective parameter change impact artifact generated for each parameter included in the proposed set of parameters.
